(19) United States
(12) Patent Application Publication
(10) Pub. No.: US 2026/0224624 A1
(43) Pub. Date: Aug. 6, 2026

(54) SWITCH RECEPTOR FOR USE IN ADOPTIVE CELL THERAPY

(71) Applicant: Oslo Universitetssykehus HF, Oslo (NO)

(72) Inventors: Jon Amund Kyte, Oslo (NO); Nicholas CASEY, Oslo (NO); Carote BECK, Oslo (NO)

(73) Assignee: Oslo Universitetssykehus HF, Oslo (NO)

(21) Appl. No.: 19/152,229

(22) PCT Filed: Feb. 1, 2024
(86) PCT No.: PCT/EP2024/052543

§ 371 Date: Jul. 30, 2025

(30) Foreign Application Priority Data
Feb. 1, 2023 (GB) 230142.0

(51) Int. Cl. A61K 35/17; A61K 40/11; A61K 40/15; A61K 40/31; A61K 40/32; A61K 40/42; A61K 40/50; A61P 35/04; C07K 14/705; C07K 14/71; C07K 14/715; C12N 5/0783; C12N 15/11; C12N 15/86

(52) U.S. Cl. CPC A61K 35/17; A61K 40/11; A61K 40/15; A61K 40/31; A61K 40/32; A61K 40/42; A61K 40/50; A61P 35/04; C07K 14/7051; C07K 14/71; C07K 14/7155; C12N 5/0636; C12N 5/0646; C12N 15/11; C12N 15/86; A61K 2239/58; C07K 2317/565; C07K 2319/02; C07K 2319/03

(57) ABSTRACT

The present invention relates chimeric receptors and their use in therapy, such as the treatment of cancer, particularly solid tumours. More particularly, the invention provides a polycistronic nucleic acid encoding: (i) a multichain chimeric receptor and; (ii) a chimeric antigen receptor (CAR) or a T-cell receptor (TCR) comprising an alpha chain and/or beta chain, wherein the multichain chimeric receptor comprises first and second polypeptides comprising extracellular domains that bind to TGFβ and endodomains comprising an intracellular signalling domain of IL-2Rβ or IL-2Rγ.

### FIELD OF THE INVENTION

The present invention relates generally to the field of chimeric receptors, transforming growth factor β (TGFβ) biology and related therapies, such as the treatment of cancer, particularly solid tumours. More particularly, the invention provides a polycistronic nucleic acid encoding a multichain chimeric receptor (a so-called “switch receptor”) and a chimeric antigen receptor (CAR) for expression in immune cells (e.g. T cells). The invention further provides vectors containing the nucleic acid molecule that may be used to modify host cells, e.g. immune cells, to express the receptors, and the use of the host cells in therapy.

### BACKGROUND TO THE INVENTION

Chimeric antigen receptors (CARs) are employed for retargeting T cells against tumour-associated antigens. CAR T cell therapy has been shown to have clinical efficacy against haematological cancers, but not yet consistently against solid tumours. The limited effect of CAR T cell therapies against solid tumours has, in part, been attributed to an immunosuppressive tumour microenvironment.

TGFβ is a key mediator of immunosuppression across most solid cancers. TGFβ inhibits T cell activity through binding to the receptors TGFβRI and TGFβRII. The binding induces hetero-dimerization of the receptors and phosphorylation of the intracellular signal mediators SMAD2 and SMAD3, which in turn induce a suppressive transcriptional programme. This leads to an attenuated T cell response, with reduced T cell proliferation, cytokine production and cytotoxicity. Further, TGFβ drives T cell differentiation into regulatory T cells.

Several CAR T cell approaches for countering TGFβ-mediated immunosuppression are being investigated. This includes a receptor that binds TGFβ via a scFv-binding domain and stimulates the engineered T cell through CD28 signalling, and a hybrid receptor fusing the TGFβ receptor II exodomain with the endodomain of 41BB.

The first approach to be brought into clinical testing is a double negative, non-signalling TGFβ receptor (dnTGFβ-R), which has been evaluated in patients with castration-resistant metastatic prostate cancer. The study showed acceptable safety and a PSA30 response in 4 of 10 patients.

TGFβ is secreted at high levels in prostate cancer, which is the most common cancer among males and a leading cause of cancer-related death. A CAR targeting the 339-amino acid cell surface protein six-transmembrane epithelial antigen of prostate-1 (STEAP1) has already been developed by the present inventors. STEAP1 is expressed in ~90% of prostate cancers and in subgroups of other cancers, such as Ewing sarcoma, lung cancer, bladder cancer, breast cancer, pancreatic cancer, glioblastoma, ovarian cancer, leukaemia, lymphoma and head and neck cancer. Across multiple cancer forms, STEAP1 has been associated with tumour proliferation, progression and invasiveness, including tumour invasion into the peritoneum. Among normal tissues, STEAP1 is mainly expressed in the prostate, which is not a vital organ. The first published trial with a STEAP1 targeting antibody-drug conjugate indicated a favourable safety profile, with no STEAP1-directed toxicity.

Nevertheless, there remains a need for improved therapies for prostate cancer and other solid tumours.

### SUMMARY OF THE INVENTION

The development of new strategies for countering the TGFβ-mediated immunosuppression of genetically modified tumour-targeted immune effector cells (e.g. CAR-T cells) for adoptive cell cancer therapy is not straightforward. The efficient generation of cells with multiple genetic modifications, e.g. the introduction of gene encoding a CAR and an additional receptor, such as the hybrid TGFβ receptor discussed above, can be problematic. In particular, the proportion of cells containing both genetic modifications may be low, meaning that additional steps may be required to select the desired cells (i.e. cells containing both receptors) and expand them to provide a therapeutically effective amount of the cells. This is critical in a clinical setting as the administration of cell population comprising cells with the capacity to expand, but that are not tumour-specific, may have undesirable side effects.

In addition, it can be difficult to predict whether multiple transgenes will be expressed at the level required to provide the desired functionality and phenotype. Even if cells containing, and successfully expressing, multiple transgenes can be generated, it is not guaranteed that the cells will maintain their function and continue to proliferate for the period required to be therapeutically effective. In this respect, it is known in the art that continued antigen stimulation of CAR-T cells may lead to T cell exhaustion or activation-induced cell death (AICD).

As discussed in detail in the Examples below, the present inventors have developed a so-called “switch receptor” that converts the immunosuppressive TGFβ signal to a stimulatory signal that promotes proliferation and survival of immune effector cells in the immunosuppressive tumour microenvironment. Notably, the inventors have shown that the TGFβ-switch receptor disclosed herein can be efficiently expressed in tandem with a tumour-targeting receptor, e.g. a chimeric antigen receptor. Moreover, and importantly, the inventors have shown that the TGFβ-switch receptor makes immune cells (e.g. T cells) resistant to TGFβ-mediated immune suppression and, unexpectedly, have demonstrated that the CAR-T cells expressing the TGFβ-switch receptor developed a phenotype associated with activation but did not show phenotypes associated with T cell exhaustion even after continued antigen stimulation. This was particularly surprising given that the TGFβ-switch receptor employs the endodomains of the shared β and γ chains between the receptors for IL-2 and IL-15 (IL-2/15 Rβγ) and IL-2 is known to promote AICD.

As the TGFβ-switch receptor is a multichain chimeric receptor, i.e. comprising two separate chimeric polypeptides, the inventors designed and developed a polycistronic transgene, which encodes both chains of the TGFβ-switch receptor and a tumour-targeting receptor, such as a CAR or T cell receptor (TCR). Advantageously, the polycistronic configuration of the transgene overcame problems associated with the low efficiency of separately inserting multiple transgenes into cells, thereby improving the proportion of cells comprising all transgenes. Moreover, the configuration exemplified in the Examples, in which the tumour-targeting receptor (e.g. CAR) is encoded downstream of the switch receptor encoding sequences, ensured that cells expressing the tumour-targeting receptor (e.g. CAR) must also contain the switch receptor genes, thereby removing the need to select cells containing all transgenes, i.e. tumour-specific cells (e.g. cells expressing the CAR) must also express the switch receptor.

While use of a polycistronic configuration increases the size of the transgene, which may be expected to impact on expression levels, the inventors found that even low expression levels were sufficient to confer the required phenotype on the transformed cells. Moreover, the inventors surprisingly found that subjecting the transformed cells to a freeze/thaw cycle followed by re-activation and expansion increased expression levels to levels commensurate with smaller transgenes. Whilst not wishing to be bound by theory, it is hypothesised that the use of a retroviral vector comprising the polycistronic transgene may explain the increase in expression following the freeze/thaw treatment. In this respect, the integration of retroviral constructs into cellular DNA depends on proliferation, and it is thought that T cells expressing the switch receptor construct may be more viable and proliferative after freezing/thawing, thereby increasing integration events which subsequently improves expression levels.

Thus, the present inventors have demonstrated that the TGFβ-switch receptor (TGFβ-multichain chimeric receptor) facilitates the activation, proliferation and functionality of immune effector cells in an immunosuppressive environment (e.g. a tumour microenvironment) without becoming exhausted and that the TGFβ-switch receptor is suitable for use in CAR T cells, and in other adoptive cell therapy approaches.

Accordingly, in one aspect the invention provides a polycistronic nucleic acid encoding: (i) a multichain chimeric receptor and; (ii) a chimeric antigen receptor (CAR) or a T-cell receptor (TCR) comprising an alpha chain and/or beta chain, wherein:
(A) the multichain chimeric receptor comprises:
(a) a first polypeptide comprising: (i) an extracellular domain comprising the extracellular domain of TGFβRI or a functional variant thereof; (ii) a transmembrane domain; and (iii) an endodomain comprising an intracellular signalling domain of IL-2Rβ or a functional variant thereof; and
(b) a second polypeptide comprising: (i) an extracellular domain comprising the extracellular domain of TGFβRII or a functional variant thereof; (ii) a transmembrane domain; and (ii) an endodomain comprising an intracellular signalling domain of IL-2Rγ or a functional variant thereof; or
(B) the multichain chimeric receptor comprises:
(a) a first polypeptide comprising: (i) an extracellular domain comprising the extracellular domain of TGFβRII or a functional variant thereof; (ii) a transmembrane domain; and (ii) an endodomain comprising an intracellular signalling domain of IL-2Rβ or a functional variant thereof; and
(b) a second polypeptide comprising: (i) an extracellular domain comprising the extracellular domain of TGFβRI or a functional variant thereof; (ii) a transmembrane domain; and (iii) an endodomain comprising an intracellular signalling domain of IL-2Rγ or a functional variant thereof.

The invention also provides a vector, preferably a viral vector (e.g. a retroviral vector), comprising the polycistronic nucleic acid disclosed herein.

A further aspect of the invention is a plurality of viral particles or virions comprising the viral vector disclosed herein.

In another aspect, the invention provides an immune effector cell:
(A) comprising the polycistronic nucleic acid or vector disclosed herein, wherein the immune effector cell expresses at its surface: (i) a multichain receptor as defined herein and (ii) a CAR or TOR as defined herein; or
(B) that expresses: (i) a multichain receptor as defined herein and (ii) an anti-STEAP-1 CAR as defined herein.

In yet another aspect, the invention provides a cell population comprising the immune effector cell disclosed herein, optionally wherein the cell population is frozen (i.e. cryopreserved) or wherein the cell population is an expanded cell population obtained from immune effector cells that have been frozen (i.e. cryopreserved) and thawed.

In a further aspect, the invention provides a pharmaceutical composition comprising the immune effector cell or the cell population disclosed herein and a pharmaceutically acceptable carrier or excipient.

In another aspect, the invention provides the immune effector cell, the cell population or the pharmaceutical composition disclosed herein, for use in therapy.

In a still further aspect the invention provides the immune effector cell, the cell population or the pharmaceutical composition disclosed herein, for use in the treatment of cancer, preferably STEAP1-positive prostate cancer.

Alternatively viewed, the invention provides a method for treating a disease or condition (e.g. cancer) in a subject, which comprises the step of administering the immune effector cell, the cell population or the pharmaceutical composition disclosed herein to the subject.

Similarly, the invention provides a method for treating cancer (e.g. STEAP1-positive prostate cancer) in a subject, which comprises the step of administering the immune effector cell, the cell population or the pharmaceutical composition disclosed herein to the subject.

In a further aspect, the invention provides a method for increasing the expression of a multichain chimeric receptor and/or a CAR or TOR at the surface of immune effector cells in a cell population, wherein the multichain chimeric receptor and the CAR are as defined herein, the method comprising:
(a) providing a cell population comprising the immune effector cell as disclosed herein, wherein the immune effector cell comprises the polycistronic nucleic acid or the vector as disclosed herein;
(b) freezing the cell population of (a);
(c) thawing the frozen cell population of (b); and
(d) activating and optionally expanding the thawed cell population of (c).

As noted above, it will be appreciated by the skilled person that the multichain TGFβ switch receptor disclosed herein may find utility in various adoptive cell therapies, particularly cancer therapies involving immune effector cells that innately display tumour cell killing activity (i.e. without modification with a tumour-targeting receptor, such as a CAR).

Accordingly, in a further aspect, the invention may be seen to provide a tumour-infiltrating lymphocyte (TIL) or allogeneic natural killer (NK) cell expressing a multichain chimeric receptor as defined herein, i.e. a modified or engineered TIL or NK cell.

Furthermore, disclosed herein is the TIL or allogeneic NK cell as described herein for use in therapy.

Further disclosed herein is the TIL or allogeneic NK cell disclosed herein for use in treating cancer, optionally wherein the cancer is selected from melanoma, non-small cell lung cancer, ovarian cancer, head and neck cancer, colorectal cancer, liver cancer, breast cancer, soft tissue sarcoma, pancreatic cancer, cervical cancer, renal cancer, gastric cancer, osteosarcoma, oesophageal cancer, glioblastoma, thyroid cancer, neuroendocrine cancer, mesothelioma, bladder cancer, prostate cancer, multiple myeloma, small cell lung cancer and endometrial cancer.

Alternatively viewed, the invention provides a method for treating and/or preventing a disease or condition in a subject (e.g. cancer), which comprises the step of administering a modified or engineered TIL or NK cell as defined above, cell population comprising said cell or pharmaceutical composition comprising said cell or cell population to the subject.

### DETAILED DESCRIPTION

The term “polycistronic” as used herein refers to a nucleic acid molecule that is or encodes a single mRNA that encodes a plurality (e.g. 2, 3, 4 or more) of proteins/polypeptides. Thus, a polycistronic gene or transgene refers to a nucleic acid molecule (e.g. DNA molecule) under the control of a single promoter and that is transcribed as a single mRNA encoding a plurality of proteins. It will be evident from the disclosures herein that the polycistronic nucleic acid molecule disclosed herein preferably is provided in the form of a vector, particularly a viral vector, preferably a retroviral vector. However, the polycistronic nucleic acid molecule disclosed herein also may be the mRNA encoded by the polycistronic gene described above.

Polycistronic genes are common in prokaryotes, e.g. operons, and it will be understood that various mechanisms may be employed to provide a polycistronic mRNA that can be translated into a plurality of separate proteins. In a representative and preferred embodiment, in the polycistronic nucleic acid disclosed herein the multichain chimeric receptor and CAR or TCR are encoded in a single open reading frame, wherein adjacent encoding sequences are separated by a sequence (i.e. an in-frame sequence, thereby maintaining the single open reading frame) encoding a self-cleaving peptide.

Self-cleaving peptides are well-known in the art and any sequence encoding a suitable self-cleaving peptide may be used in the polycistronic nucleic acid disclosed herein. A suitable self-cleaving peptide refers to any peptide that facilitates or enables the production of a plurality of proteins from a polycistronic RNA during or after translation of the proteins. Notably, a self-cleaving peptide does not require the introduction of external factors to enable cleavage or separate production of the proteins to occur.

The class of 2A self-cleaving peptides (also known as 2A peptides) are routinely used in the art. The 2A peptides typically are 18-22 amino acids long and are derived from viruses. The 2A peptides contain a core consensus motif of DXEXNPGP (SEQ ID NO: 1), wherein X is any amino acid. While the exact molecular mechanism of 2A-peptide-mediated cleavage is still unknown, it is thought to be induced by ribosomal “skipping” of the peptide bond between the C-terminal glycine and proline residues.

The polycistronic nucleic acid molecule disclosed herein may comprise or consist of three polypeptide encoding sequences separated by sequences encoding self-cleaving peptides, e.g. 2A peptides. Thus, the polycistronic nucleic acid may be a tricistronic nucleic acid. However, it will be evident that additional encoding sequences may be included, e.g. where it is desirable to encode multiple chains of a TOR, e.g. alpha and beta chains, or where the CAR is a multichain CAR. Moreover, it may be advantageous to express the multichain receptor and/or CAR with a safety switch (also known as a suicide marker), such as an RQR8 safety switch. Thus, the polycistronic nucleic acid may be a tetracistronic nucleic acid.

The self-cleaving peptides encoded by the polycistronic nucleic acid may be a 2A peptide. In particular, each self-cleaving peptide encoded by the polycistronic nucleic acid may be independently selected from the group consisting of T2A, P2A, E2A and F2A or a functional variant thereof, preferably the group consisting of T2A and P2A or a functional variant thereof. For instance, the first self-cleaving peptide, i.e. the peptide between the first and second polypeptides encoded by the polycistronic nucleic acid may be a T2A peptide or functional variant thereof, and the second self-cleaving peptide, i.e. the peptide between the second and third polypeptides encoded by the polycistronic nucleic acid may be a P2A peptide or functional variant thereof. The sequences of some 2A peptides are provided in Table 1.

TABLE 1

| 2A peptide | Sequence | SEQ ID NO: |
|---|---|---|
| T2A | GSGEGRGSLLTCGDVEENPGP | 2 |
| P2A | GSGATNFSLLKQAGDVEENPGP | 3 |
| E2A | GSGQCTNYALLKLAGDVESNPGP | 4 |
| F2A | GSGVKQTLNFDLLKLAGDVESNPGP | 5 |

Notably, the sequences provided in Table 1 include an N-terminal GSG sequence that is not present in the native peptides. The additional sequence is thought to improve the efficacy of the peptides. Thus, the GSG sequence may be omitted from the self-cleaving 2A peptide encoded by the polycistronic nucleic acid.

The term “functional variant” in the context of self-cleaving peptides refers to variants of the 2A peptides (e.g. SEQ ID NOs: 2-5) that are capable of functioning as self-cleaving peptides (e.g. inducing by ribosomal “skipping” of the peptide bond between the C-terminal glycine and proline residues) with the same, similar or greater efficiency as the reference peptides (e.g. native peptides, optionally including the N-terminal GSG sequence). Thus, a functional variant may comprise an amino acid sequence with at least 80% sequence identity (e.g. 85%, 90% or 90% sequence identity) to an amino acid sequence as set forth in SEQ ID NO: 2-5, preferably wherein the functional variant contains the consensus sequence of SEQ ID NO: 1. Thus, the functional variant of the 2A peptide functions to enable the separate production (translation) of proteins encoded by the polycistronic nucleic acid.

By “similar efficiency” is meant that the level of cleavage achieved by the functional variant is comparable to the corresponding reference 2A peptide, e.g. is not more than a factor of 10 different. More preferably the difference between the levels of cleavage is less than a factor of 8, preferably less than a factor of 5, 4, 3 or 2.

As the 2A peptides are derived from viral genomes, it may be advantageous to codon-optimise the encoding sequences for expression in the cell(s) or organism of interest, e.g. human cells. Thus, the nucleotide sequences encoding the self-cleaving peptides (e.g. 2A peptides) in the polycistronic nucleic acid may be codon-optimised sequences, preferably codon-optimised for expression in human cells, particularly human immune cells.

It will be understood that the same 2A peptide or functional variant thereof may be used between each polypeptide encoded by the polycistronic nucleic acid. However, to minimise the risk of recombination events within the polycistronic nucleic acid, it is preferred that the nucleotide sequence encoding each self-cleaving peptide is different. In view of the degeneracy of the genetic code however, it is possible that the same 2A peptide or functional variant thereof may be encoded by different nucleotide sequences. Thus, for instance, the first and second self-cleaving peptide encoding sequences may both encode the same amino acid sequence selected from one of SEQ ID NO: 2-5, but may have different nucleotide sequences.

The inventors have determined that it may be particularly advantageous to maximise the differences between the nucleotide sequences encoding the self-cleaving peptides. This may be achieved by using nucleotide sequences that encode different self-cleaving peptide amino acid sequences. Thus, the amino acid sequence of each self-cleaving peptide may be different. For example, the first self-cleaving peptide encoding sequence may encode a first self-cleaving peptide amino acid sequence (e.g. T2A) and the second self-cleaving peptide encoding sequence may encode a second self-cleaving peptide amino acid sequence (e.g. P2A or a functional variant thereof or a functional variant of T2A).

In the polycistronic nucleic acid exemplified in the Examples, the codon-optimised sequences encoding the self-cleaving peptides were manually modified to increase the sequence variance between the sequences (i.e. to decrease the sequence identity between the sequences). Whilst not wishing to bound by theory, it is thought that the self-cleaving peptide nucleotide sequences used in the exemplified polycistronic nucleic acid may contribute to its stability and expression. Thus, in some embodiments, the nucleotide sequences encoding the self-cleaving peptides in the polycistronic nucleic acid have less than about 80% sequence identity, preferably less than about 75%, 70%, 65% or 60% sequence identity. In some embodiments, the polycistronic nucleic acid comprises a nucleotide sequence encoding a self-cleaving peptide of SEQ ID NO: 6 (GGCTCAGGAGAAGGCAGGGGCTCCCTGCTCACATGCGGGGATGTGGAAGAG AACCCAGGACCA) and SEQ ID NO: 7 (GGAAGTGGCGCCACCAATTTCTCATTGCTTAAGCAGGCCGGGGACGTGGAG GAAAACCCAGGGCCC). It will be evident that the positions (i.e. order) of the sequences may not be critical. However, in a representative embodiment SEQ ID NO: 6 is between the sequences encoding the first and second polypeptides in the polycistronic nucleic acid and SEQ ID NO: 7 is between the sequences encoding the second and third polypeptides in the polycistronic nucleic acid.

It is thought that the order of the polypeptide encoding sequences within the polycistronic nucleic acid is not critical. In particular, the encoding sequences of the polypeptide chains of the multichain chimeric receptor may be in any order. For instance, in some embodiments, the first polypeptide of the multichain chimeric receptor is encoded upstream of the second polypeptide encoded by the multichain chimeric receptor, i.e. at or towards the 5′ end of the polycistronic nucleic acid. In other words, in some embodiments the first polypeptide of the multichain chimeric receptor is the first polypeptide encoded by the polycistronic nucleic acid. In some embodiments, the second polypeptide of the multichain chimeric receptor is encoded upstream of the first polypeptide encoded by the multichain chimeric receptor, i.e. at or towards the 5′ end of the polycistronic nucleic acid. In other words, in some embodiments the second polypeptide of the multichain chimeric receptor is the first polypeptide encoded by the polycistronic nucleic acid.

It will be evident that the nucleotide sequence encoding the tumour-targeting receptor, i.e. CAR or TCR, may be at any position within the polycistronic nucleic acid, e.g. at or towards the 5′ end of the polycistronic nucleic acid, between the sequences encoding the first and second polypeptides of the multichain chimeric receptor or at or towards 3′ end of the polycistronic nucleic acid. However, as noted above, it may be advantageous for the nucleotide sequence encoding the tumour-targeting receptor, i.e. CAR or TCR, to be downstream of the nucleotide sequences encoding the polypeptide chains of the multichain chimeric receptor to ensure that cells expressing the tumour-targeting receptor (e.g. CAR) also contain the multichain chimeric receptor encoding sequences, thereby removing the need to select cells containing all transgenes, i.e. tumour-specific cells (e.g. cells expressing the CAR) must also express the multichain chimeric receptor.

Thus, the polycistronic nucleic acid may encode in order from 5′ to 3′:
(a) the first polypeptide of the multichain chimeric receptor;
(b) a first self-cleaving peptide (e.g. T2A or a functional variant thereof);
(c) the second polypeptide of the multichain chimeric receptor;
(d) a second self-cleaving peptide (e.g. P2A or a functional variant thereof); and
(e) the CAR or the TCR alpha chain or beta chain; and
optionally when the nucleic acid encodes a TOR alpha chain and beta chain
(f) a third self-cleaving peptide; and
(g) the TCR chain not encoded in (e).

Similarly, the polycistronic nucleic acid may encode in order from 5′ to 3′:
(a) the second polypeptide of the multichain chimeric receptor;
(b) a first self-cleaving peptide (e.g. T2A or a functional variant thereof);
(c) the first polypeptide of the multichain chimeric receptor;
(d) a second self-cleaving peptide (e.g. P2A or a functional variant thereof); and
(e) the CAR or the TOR alpha chain or beta chain; and
optionally when the nucleic acid encodes a TOR alpha chain and beta chain
(f) a third self-cleaving peptide; and
(g) the TCR chain not encoded in (e).

In a preferred aspect, the polycistronic nucleic acid may encode in order from 5′ to 3′:
(a) the first polypeptide of the multichain chimeric receptor;
(b) a first self-cleaving peptide (e.g. T2A or a functional variant thereof);
(c) the second polypeptide of the multichain chimeric receptor;
(d) a second self-cleaving peptide (e.g. P2A or a functional variant thereof); and
(e) the CAR.

More particularly, the polycistronic nucleic acid may encode:
(A) a multichain chimeric receptor comprising:
(a) a first polypeptide comprising: (i) an extracellular domain comprising the extracellular domain of TGFβRI or a functional variant thereof; (ii) a transmembrane domain; and (iii) an endodomain comprising an intracellular signalling domain of IL-2Rβ or a functional variant thereof; and
(b) a second polypeptide comprising: (i) an extracellular domain comprising the extracellular domain of TGFβRII or a functional variant thereof; (ii) a transmembrane domain; and (iii) an endodomain comprising an intracellular signalling domain of IL-2Rγ or a functional variant thereof; and
(B) a CAR comprising: (i) an extracellular domain comprising an antigen binding domain; (ii) a transmembrane domain; and (iii) an endodomain comprising intracellular signalling domain.

The transmembrane domain of the first polypeptide may comprise or consist of the transmembrane domain of IL-2Rβ or a functional variant thereof and/or the transmembrane domain of the second polypeptide may comprise or consist of the transmembrane domain of IL-2Rγ or a functional variant thereof.

Thus, the polycistronic nucleic acid may encode:
(A) a multichain chimeric receptor comprising:
(a) a first polypeptide comprising: (i) an extracellular domain comprising the extracellular domain of TGFβRI or a functional variant thereof; (ii) a transmembrane domain comprising or consisting of the transmembrane domain of IL-2Rβ or a functional variant thereof; and (iii) an endodomain comprising an intracellular signalling domain of IL-2Rβ or a functional variant thereof; and
(b) a second polypeptide comprising: (i) an extracellular domain comprising the extracellular domain of TGFβRII or a functional variant thereof; (ii) a transmembrane domain comprising or consisting of the transmembrane domain of IL-2Rγ or a functional variant thereof; and (iii) an endodomain comprising an intracellular signalling domain of IL-2Rγ or a functional variant thereof; and
(B) a CAR comprising: (i) an extracellular domain comprising an antigen binding domain; (ii) a transmembrane domain; and (iii) an endodomain comprising intracellular signalling domain.

It will be evident that in the configurations set out above, the CAR may be replaced by a TOR alpha and/or beta chain as defined herein and such configurations form embodiments of the invention.

The term “multichain chimeric receptor” refers to a receptor protein comprising at least two (chimeric) polypeptide chains which are associated or bound to each other via sites within the polypeptide chains which allow binding or association, e.g. via the extracellular domains, transmembrane domains and/or endodomains. Multichain chimeric receptors may dimerise primarily via their interaction with their cognate ligand, i.e. TGFβ, and/or innately via other domains, e.g. transmembrane domains.

The extracellular domain or exodomain of the first polypeptide of the multichain chimeric receptor contains at least the TGFβ binding domain of TGFβRI or a functional variant thereof, e.g. the extracellular domain of TGFβRI or a functional domain thereof, or at least the TGFβ binding domain of TGFβRII or a functional variant thereof, e.g. the extracellular domain of TGFβRII or a functional domain thereof. It will be appreciated that for the multichain chimeric receptor to function, it must contain an extracellular domain of TGFβRI and TGFβRII, one on each chain. Accordingly, when the first polypeptide of the multichain chimeric receptor contains at least the TGFβ binding domain of TGFβRI or a functional variant thereof, the second polypeptide of the multichain chimeric receptor contains at least the TGFβ binding domain of TGFβRII or a functional variant thereof. Similarly, when the first polypeptide of the multichain chimeric receptor contains at least the TGFβ binding domain of TGFβRII or a functional variant thereof, the second polypeptide of the multichain chimeric receptor contains at least the TGFβ binding domain of TGFβRI or a functional variant thereof.

Human TGFβRI comprises an exodomain of 126 amino acids (SEQ ID NO: 8) which comprises at least two domains: a signal or leader sequence (amino acids 1-33 of SEQ ID NO: 8); and a ligand binding domain (amino acids 34-126 of SEQ ID NO: 8). It will be appreciated that signal sequence of the TGFβRI exodomain may be substituted with any other suitable signal sequence that functions to target the first polypeptide of the multichain chimeric receptor to its site of action, i.e. the extracellular membrane of an immune cell.

Thus, the exodomain of the first or second polypeptide of the multichain chimeric receptor of the invention comprises at least the ligand binding domain of the TGFβRI exodomain (which may comprise amino acid residues 34-126 of SEQ ID NO: 8) or a functional variant thereof. Thus, the exodomain may consist of the ligand binding domain of the TGFβRI exodomain (amino acid residues 34-126 of SEQ ID NO: 8) or a functional variant thereof and may comprise additional sequence, i.e. a signal sequence. It will be appreciated that signal sequence may be the native sequence of the TGFβRI or a functional variant thereof.

Accordingly, in preferred embodiments, the exodomain of the first or second polypeptide of the multichain chimeric receptor of the invention comprises the whole of the TGFβRI exodomain or a functional variant thereof, e.g. an amino acid sequence as set forth in SEQ ID NO: 8 or a functional variant thereof. The functional variant typically comprises an amino acid sequence with at least 80% (e.g. at least 85%, 90%, 95%, 96%, 97%, 98% or 99%) sequence identity to an amino acid sequence as set forth in SEQ ID NO: 8.

Human TGFβRII comprises an exodomain of 166 amino acids (SEQ ID NO: 9) which comprises at least two domains: a signal or leader sequence (amino acids 1-22 of SEQ ID NO: 9); and a ligand binding domain (amino acids 23-166 of SEQ ID NO: 9). It will be appreciated that signal sequence of the TGFβRII exodomain may be substituted with any other suitable signal sequence that functions to target the first polypeptide of the multichain chimeric receptor to its site of action, i.e. the extracellular membrane of an immune cell.

Thus, the exodomain of the second or first polypeptide of the multichain chimeric receptor of the invention comprises at least the ligand binding domain of the TGFβRII exodomain (which may comprise amino acid residues 23-166 of SEQ ID NO: 9) or a functional variant thereof. Thus, the exodomain may consist of the ligand binding domain of the TGFβRII exodomain (amino acid residues 23-166 of SEQ ID NO: 9) or a functional variant thereof and may comprise additional sequence, i.e. a signal sequence. It will be appreciated that signal sequence may be the native sequence of the TGFβRII or a functional variant thereof.

Accordingly, in preferred embodiments, the exodomain of the second or first polypeptide of the multichain chimeric receptor of the invention comprises the whole of the TGFβRII exodomain or a functional variant thereof, e.g. an amino acid sequence as set forth in SEQ ID NO: 9 or a functional variant thereof. The functional variant typically comprises an amino acid sequence with at least 80% (e.g. at least 85%, 90%, 95%, 96%, 97%, 98% or 99%) sequence identity to an amino acid sequence as set forth in SEQ ID NO: 9.

The endodomain of the first polypeptide of the multichain chimeric receptor contains an intracellular signalling domain of IL-2Rβ or a functional variant thereof, e.g. the endodomain of IL-2Rβ or a functional domain thereof.

Human IL-2Rβ comprises an endodomain of 286 amino acids (SEQ ID NO: 10) which comprises various signalling domains involved IL-2 signalling, particularly JAK1 and JAK2 binding motifs that function to initiate the JAK-STAT signalling pathway upon ligand binding and receptor dimerization in native IL2Rβ.

Thus, the endodomain of the first polypeptide of the multichain chimeric receptor of the invention may comprise at least the intracellular signalling domain of IL-2Rβ endodomain that is necessary and sufficient to initiate one or more signalling pathways associated with the native IL-2Rβ polypeptide, e.g. the JAK-STAT signalling pathway, or a functional variant thereof (e.g. an intracellular signalling domain comprising a JAK-1 and/or JAK-2 binding motif). Thus, the endodomain may comprise or consist of the intracellular signalling domain defined above or a functional variant thereof.

Accordingly, in preferred embodiments, the endodomain of the first polypeptide of the multichain chimeric receptor of the invention comprises the whole of the IL-2Rβ endodomain or a functional variant thereof, e.g. an amino acid sequence as set forth in SEQ ID NO: 10 or a functional variant thereof. The functional variant typically comprises an amino acid sequence with at least 80% (e.g. at least 85%, 90%, 95%, 96%, 97%, 98% or 99%) sequence identity to an amino acid sequence as set forth in SEQ ID NO: 10.

The endodomain of the second polypeptide of the multichain chimeric receptor contains an intracellular signalling domain of IL-2Rγ or a functional variant thereof, e.g. the endodomain of IL-2Rγ or a functional domain thereof.

Human IL-2Rγ comprises an endodomain of 86 amino acids (SEQ ID NO: 11) which comprises various signalling domains involved IL-2 signalling, particularly a JAK3 binding motif that functions to initiate the JAK-STAT signalling pathway upon ligand binding and receptor dimerization in native IL2Rγ.

Thus, the endodomain of the second polypeptide of the multichain chimeric receptor of the invention may comprise at least the intracellular signalling domain of IL-2Rγ endodomain that is necessary and sufficient to initiate one or more signalling pathways associated with the native IL-2Rγ polypeptide, e.g. the JAK-STAT signalling pathway, or a functional variant thereof (e.g. an intracellular signalling domain comprising a JAK-3 binding motif). Thus, the endodomain may comprise or consist of the intracellular signalling domain defined above or a functional variant thereof.

Accordingly, in preferred embodiments, the endodomain of the second polypeptide of the multichain chimeric receptor of the invention comprises the whole of the IL-2Rγ endodomain or a functional variant thereof, e.g. an amino acid sequence as set forth in SEQ ID NO: 11 or a functional variant thereof. The functional variant typically comprises an amino acid sequence with at least 80% (e.g. at least 85%, 90%, 95%, 96%, 97%, 98% or 99%) sequence identity to an amino acid sequence as set forth in SEQ ID NO: 11.

The term “intracellular signalling domain” refers to the parts of the multichain chimeric receptor that participate in transducing the message of effective multichain chimeric receptor binding to TGFβ into the interior of a particular cell, e.g. an immune cell, to elicit cell function, e.g. activation, cytokine production, proliferation or other cellular responses elicited with ligand binding to the exodomain(s) of the multichain chimeric receptor. The intracellular signalling domains are comprised within the endodomain of the receptor. The term “cell function” refers to a specialized function of a cell. Cell function of a T cell, for example, may be cytolytic activity or help, activity including the secretion of a cytokine or cytotoxic activity. Thus, the term “intracellular signalling domain” refers to the portion of a protein that transduces a function signal and that directs a cell to perform a specialized function. While the entire intracellular signalling domain of a protein can be employed, in many cases it is not necessary to use the entire domain. To the extent that a truncated portion of an intracellular signalling domain is used, such truncated portion may be used in place of the entire domain as long as it transduces the effector function signal. The term intracellular signalling domain therefore includes any truncated portion of the IL2Rβ and IL2Rγ intracellular signalling domains described above that are sufficient to transduce a function signal. It will be appreciated that the multichain chimeric receptor may be introduced into cells, such as precursor or progenitor cells where the intracellular signalling domain may not be capable of inducing a cell function. However, the multichain chimeric receptor should be capable of signalling once expressed within an appropriate cell type, e.g. an immune cell, e.g. a T cell.

The multichain chimeric receptor comprises transmembrane proteins. Accordingly, each chain of the multichain chimeric receptor comprises a transmembrane domain (a membrane-spanning domain) connecting the exo- and endodomains. It will be appreciated that any suitable transmembrane domain may be used to connect the exo- and endodomains of the chains of the multichain chimeric receptor. It is sufficient that the transmembrane domain enables the activation of the intracellular signalling domain(s) in the endodomains of the polypeptide chains upon ligand (TGFβ) binding. Conveniently, the transmembrane domain may be obtained from the same protein as the exo- or endodomain, although this is not essential. In a preferred aspect, the transmembrane domain may be obtained from the same protein as the endodomain, i.e. the transmembrane domain of the first polypeptide is obtained from IL-2Rβ and the transmembrane domain of the second polypeptide is obtained from IL-2Rγ.

Thus, the transmembrane domain of the first polypeptide may comprise or consist of the transmembrane domain of IL-2Rβ or a functional variant thereof. More particularly, the transmembrane domain of the first polypeptide may comprise or consist of an amino acid sequence as set forth in SEQ ID NO: 12 or a functional variant thereof. The functional variant typically comprises an amino acid sequence with at least 80% (e.g. at least 85%, 90%, 95%, 96%, 97%, 98% or 99%) sequence identity to an amino acid sequence as set forth in SEQ ID NO: 12.

The transmembrane domain of the second polypeptide may comprise or consist of the transmembrane domain of IL-2Rγ or a functional variant thereof. More particularly, the transmembrane domain of the second polypeptide may comprise or consist of an amino acid sequence as set forth in SEQ ID NO: 13 or a functional variant thereof. The functional variant typically comprises an amino acid sequence with at least 80% (e.g. at least 85%, 90%, 95%, 96%, 97%, 98% or 99%) sequence identity to an amino acid sequence as set forth in SEQ ID NO: 13.

The term “functional variant” refers to variants of the polypeptides and domains disclosed herein that have an equivalent or greater activity relative to the reference polypeptide. It will be evident that functional variants retain the functional activity of the reference polypeptides (i.e. are functionally equivalent) and may be sequence variants and/or portions of the reference polypeptides. For instance, the functional variants of the TGFβRI and TGFβRII exodomains or the ligand binding domains thereof function to enable the multichain chimeric receptor to bind specifically to TGFβ, and preferably have the same, similar or greater affinity of the native polypeptides. Similarly, the functional variants of the IL-2Rβ and γ endodomains function to transduce a function signal and that directs a cell to perform a specialized function with the same, similar or greater efficacy of the native polypeptides.

By “similar affinity” is meant that the binding affinity of the functional variant for TGFβ is comparable to the TGFβRI and TGFβRII exodomains, e.g. is not more than a factor of 20 different. More preferably the difference between the binding affinities is less than a factor of 15, more preferably less than a factor of 10, most preferably less than a factor of 5, 4, 3 or 2.

By “similar efficacy” is meant that the intracellular signal provided by the functional variant of the IL2Rβ and γ endodomains is comparable to the IL2Rβ and γ endodomains, e.g. is not more than a factor of 20 different. More preferably the difference between the binding affinities is less than a factor of 15, more preferably less than a factor of 10, most preferably less than a factor of 5, 4, 3 or 2.

A functional portion may comprise at least 50% of the polypeptide or domain from which it is derived, e.g. 50-99%, 55-95%, 60-90%, 65-85%, such as about at least 55%, 60%, 65%, 70%, 75%, 80%, 85%, 90% or 95% of the polypeptide or domain from which it is derived, as long as it retains the required functional activity, e.g. it is functionally equivalent to the full-length polypeptide or domain from which it is derived.

Thus, in a preferred embodiment, the polycistronic nucleic acid encodes: (i) a multichain chimeric receptor and; (ii) a chimeric antigen receptor (CAR) or a T-cell receptor (TCR) comprising an alpha chain and/or beta chain, wherein:
(A) the multichain chimeric receptor comprises:
(a) a first polypeptide comprising: (i) an extracellular domain comprising the extracellular domain of TGFβRI having an amino acid sequence as set forth in SEQ ID NO: 8 or a functional variant thereof having an amino acid sequence with at least 80% (e.g. at least 85%, 90%, 95%, 96%, 97%, 98% or 99%) sequence identity to an amino acid sequence as set forth in SEQ ID NO: 8; (ii) a transmembrane domain comprising or consisting of the transmembrane domain of IL-2Rβ having an amino acid sequence as set forth in SEQ ID NO: 12 or a functional variant thereof having an amino acid sequence with at least 80% (e.g. at least 85%, 90%, 95%, 96%, 97%, 98% or 99%) sequence identity to an amino acid sequence as set forth in SEQ ID NO: 12; and (iii) an endodomain comprising an intracellular signalling domain of IL-2Rβ having an amino acid sequence as set forth in SEQ ID NO: 10 or a functional variant thereof having an amino acid sequence with at least 80% (e.g. at least 85%, 90%, 95%, 96%, 97%, 98% or 99%) sequence identity to an amino acid sequence as set forth in SEQ ID NO: 10; and
(b) a second polypeptide comprising: (i) an extracellular domain comprising the extracellular domain of TGFβRII having an amino acid sequence as set forth in SEQ ID NO: 9 or a functional variant thereof having an amino acid sequence with at least 80% (e.g. at least 85%, 90%, 95%, 96%, 97%, 98% or 99%) sequence identity to an amino acid sequence as set forth in SEQ ID NO: 9; (ii) a transmembrane domain comprising or consisting of the transmembrane domain of IL-2Rγ having an amino acid sequence as set forth in SEQ ID NO: 13 or a functional variant thereof having an amino acid sequence with at least 80% (e.g. at least 85%, 90%, 95%, 96%, 97%, 98% or 99%) sequence identity to an amino acid sequence as set forth in SEQ ID NO: 13; and (iii) an endodomain comprising an intracellular signalling domain of IL-2Rγ having an amino acid sequence as set forth in SEQ ID NO: 11 or a functional variant thereof having an amino acid sequence with at least 80% (e.g. at least 85%, 90%, 95%, 96%, 97%, 98% or 99%) sequence identity to an amino acid sequence as set forth in SEQ ID NO: 11.

In a particularly preferred embodiment, the first polypeptide comprises an amino acid sequence as set forth in SEQ ID NO: 14 or functional variant thereof comprising an amino acid sequence with at least 80% sequence identity to an amino acid sequence as set forth in SEQ ID NO: 14, wherein the functional variant is capable of binding to TGFβ and transducing a IL-2 function signal when expressed in a suitable cell, e.g. an immune cell, such as a T cell, with a functional second polypeptide of the multichain receptor.

In a further particularly preferred embodiment, the second polypeptide comprises an amino acid sequence as set forth in SEQ ID NO: 15 or functional variant thereof comprising an amino acid sequence with at least 80% sequence identity to an amino acid sequence as set forth in SEQ ID NO: 15, wherein the functional variant is capable of binding to TGFβ and transducing a IL-2 function signal when expressed in a suitable cell, e.g. an immune cell, such as a T cell, with a functional first polypeptide of the multichain receptor.

It will be evident that in order to achieve the functional requirements described above, the first and second polypeptides must be expressed at the surface of a cell, e.g. an immune cell, i.e. they must contain a functional signal sequence. Thus, this represents a further functional feature of the functional variant polypeptides described above.

The term “chimeric receptor” refers to a receptor protein comprising linked domains from two or more proteins, e.g. an exodomain from a first protein and an endodomain from a second protein. Typically, at least one of the domains is derived from a receptor protein. Thus, a chimeric receptor may be viewed as an “engineered receptor” and these terms are used interchangeably herein.

A “Chimeric antigen receptor”, “CAR” or “CAR construct” refers to engineered receptors which can confer an antigen specificity onto cells (e.g. immune cells, such as T cells). A CAR may comprise a single polypeptide chain or may comprise two or more polypeptide chains (e.g. a first polypeptide chain and a second polypeptide chain). In particular, a CAR enables a cell to bind specifically to a particular antigen, e.g. a target molecule such as a target protein, whereupon a signal is generated by the endodomain (comprising an intracellular signalling domain) of the CAR, e.g. a signal resulting in activation of the cell. CARs are also known as artificial T-cell receptors, chimeric T-cell receptors or chimeric immunoreceptors. In particular, the CAR encoded by the polycistronic nucleic acid may function to confer cells expressing the receptor (e.g. T cells) with the ability to bind specifically to tumour associated antigens. Accordingly, the antigen binding domain of the CAR may bind to (have affinity for) a tumour associated antigen.

The structure of CARs is well-known in the art and several generations of CARs have been produced. For instance, as a minimum a CAR may contain an extracellular antigen-specific targeting region or antigen binding domain, which is or forms part of the exodomain (also known as the extracellular domain or ectodomain) of the CAR, a transmembrane domain, and an intracellular signalling domain (which is or is comprised within an endodomain). However, the CAR may contain further domains to improve its functionality, e.g. one or more co-stimulatory domains to improve T cell proliferation, cytokine secretion, resistance to apoptosis, and in vivo persistence. As discussed above, the CAR may comprise more than one polypeptide chain and thus the domains may occur within the same or within different polypeptides, typically which associate with one another. Thus, a CAR may comprise two polypeptides wherein the first polypeptide comprises the extracellular domain, a transmembrane domain and optionally an endodomain, and the second polypeptide comprises an endodomain and optionally a transmembrane domain. However, in a preferred aspect, the CAR encoded by the polycistronic nucleic acid consists of a single polypeptide, i.e. comprising an extracellular domain, a transmembrane domain and an endodomain.

Thus, a CAR construct generally comprises an antigen binding domain, optionally a hinge domain, which functions as a spacer to extend the antigen binding domain away from the plasma membrane of the cell (e.g. immune cell) on which it is expressed, a transmembrane domain, an intracellular signalling domain (e.g. the signalling domain from the zeta chain of the CD3 molecule (CD32) of the TOR complex, or an equivalent) and optionally one or more co-stimulatory domains, which may assist in signalling or functionality of the cell expressing the CAR. A CAR may also comprise a signal or leader sequence or domain which functions to target the protein to the membrane and may form part of the exodomain of the CAR. The different domains may be linked directly or by linkers, and/or may occur within different polypeptides, e.g. within two polypeptides which associate with one another. A variety of options are available for these different domains and linkers as discussed in detail below.

A T cell receptor (TCR) is a disulfide-linked membrane-anchored heterodimeric protein typically consisting of variable alpha (α) and beta (β) chains expressed as part of a complex with the invariant CD3 chain molecules. The alpha (α) and beta (β) chains associate with six additional adaptor proteins to form an octameric complex. The complex contains both α and β chains, which form the ligand-binding site of the receptor, and the signalling modules CD3δ, CD3γ, CD3ε and CD3ζ.

The TOR α- and β-chains each have three hypervariable or complementarity-determining regions (CDRs). The residues in these variable domains are located in two regions of the TCR, at the interface of the α- and β-chains and in the β-chain framework region that is thought to be in proximity to the CD3 signal-transduction complex. CDR3 is the main CDR responsible for recognizing processed antigen.

As the TCR α- and β-chains form the ligand/antigen binding domain of the TCR, genes encoding these chains can be isolated from T cells that bind to specific antigens and heterologously expressed in other T cells to confer specific antigen binding on the engineered cells. Thus, TCR α- and/or β-chains can be used to redirect T cells to a particular target antigen akin to a CAR as described above, e.g. a tumour associated antigen. This may be particularly effective in T cells that do not express endogenous alpha and/or beta chains, e.g. in cells in which the endogenous alpha and/or beta chains have been inactivated, e.g. knocked-out. Accordingly, the TOR alpha and/or beta chains encoded by the polycistronic nucleic acid function to confer cells expressing the receptor (e.g. T cells) with the ability to bind specifically to tumour associated antigens. Accordingly, the antigen binding domain(s) of the TCR alpha and/or beta chains may bind to (have affinity for) a tumour associated antigen.

STEAP1 is highly expressed in prostate cancer cells and up-regulated in multiple other cancer forms (lung cancer, bladder cancer, Ewing sarcoma, breast cancer, pancreatic cancer, glioblastoma, ovarian cancer, leukaemia, lymphoma, head and neck cancer). In normal tissues, STEAP1 is mainly expressed in the prostate, which is a dispensable organ.

HER2 is highly expressed in 15-30% of breast cancers and up-regulated in multiple some ovarian cancers and gastric cancers.

Thus, the tumour associated antigen to which the CAR and/or TCR binds may be an antigen that is up-regulated in any one or more of prostate cancer, Ewing sarcoma, lung cancer, bladder cancer, breast cancer, pancreatic cancer, glioblastoma, ovarian cancer, leukaemia, lymphoma and head and neck cancer. Accordingly, the tumour associated antigen may STEAP-1 or HER2, preferably STEAP-1. Furthermore, the CAR may be an anti-STEAP-1 or anti-HER2 CAR (preferably an anti-STEAP-1 CAR) or the TCR may be an anti-STEAP-1 TCR or an anti-HER2 TCR (preferably an anti-STEAP-1 TCR). Alternatively viewed, the CAR or TCR may be directed against STEAP1 or HER2, preferably STEAP-1.

The term “directed against STEAP1” is synonymous with “specific for STEAP1” or “anti-STEAP1”, that is it means simply that the CAR or TCR is capable of binding specifically to STEAP1. Similarly, the term “directed against HER2” is synonymous with “specific for HER2” or “anti-HER2”, that is it means simply that the CAR or TCR is capable of binding specifically to HER2. In particular, the antigen-binding domain of the CAR or TCR is capable of binding specifically to STEAP1 or HER2 (more particularly when the CAR or TCR is expressed on the surface of an immune effector cell). Specific binding may be distinguished from non-specific binding to a non-target antigen (in this case an antigen other than STEAP1 or HER2). Thus, an immune effector cell expressing the CAR or TOR may redirected to bind specifically to and exhibit cytotoxicity to (e.g. kill) a STEAP1-expressing or HER2-expressing target cell. Alternatively expressed, the immune effector cell may be modified to redirect cytotoxicity towards target cells expressing STEAP1 or HER2.

When the CAR or TOR herein are expressed on the surface of immune cells the CAR or TCR are capable of binding to STEAP1 expressed on a target cell surface. Thus, such immune cells may be directed to prostate cancer cells and provide cytotoxic activity based on an accessible target epitope on STEAP1.

Alternatively, when the CAR or TOR herein are expressed on the surface of immune cells the CAR or TCR are capable of binding to HER2 expressed on a target cell surface. Thus, such immune cells may be directed to breast cancer cells and provide cytotoxic activity based on an accessible target epitope on HER2.

A single chain Fv (scFv) is a preferred antigen-binding domain for use in the CAR disclosed herein. As used herein, an antigen binding domain (e.g. scFv) is a protein moiety able to bind an extracellular target epitope under physiological conditions, in particular physiological conditions in a tumour environment. The antigen-binding domains (e.g. scFvs) used in the CAR herein may be able to bind to STEAP1. The antigen-binding domain of the CAR (e.g. the scFv) comprises two sequences; one variable domain from an antibody light chain (V_{L}) and one variable domain from an antibody heavy chain (V_{H}). A variety of anti-STEAP1 antibodies were screened for specific binding to prostate cancer cells, and the scFv used in the CAR disclosed herein was selected based on the antibody demonstrating the best binding.

In one embodiment, the scFv comprises or consists of, from N-terminus to C-terminus, V_{H}-linker-V_{L}. In another embodiment, the scFv comprises or consists of, from N-terminus to C-terminus, V_{L}-linker-V_{H}. The linker has a certain (sufficient) length in order to allow the V_{H} and V_{L} sequences to form a functional antigen-binding domain. In one embodiment, the linker comprises 10 to 30 amino acid residues. In one embodiment, the linker comprises 15 to 25 glycine and/or serine residues. Exemplary flexible linkers include glycine polymers (G) n, glycine-serine polymers, where n is an integer of at least one, glycine-alanine polymers, alanine-serine polymers, and other flexible linkers known in the art. Glycine and glycine-serine polymers are relatively unstructured, and therefore may be able to serve as a neutral tether between domains of fusion proteins such as the CARs described herein. In particular, the linker may comprise a number of “G4S” repeats, in which the amino acid sequence motif “GGGGS” (SEQ ID NO: 16) is repeated a certain number of times. In one embodiment, the linker is represented by the sequence GGGGSGGGGSGGGGSGGGGS (SEQ ID NO: 17).

Each V_{L} and V_{H} comprises three complementarity determining regions (CDRs) flanked by framework sequences. The antigen-binding domain (e.g. scFv) may comprise:
a V_{L} comprising CDR1, CDR2 and CDR3 represented by KSSQSLLYRSNQKNYLA (SEQ ID NO: 18), WASTRES (SEQ ID NO: 19) and QQYYNYPRT (SEQ ID NO: 20), respectively;
and
a V_{H} comprising CDR1, CDR2 and CDR3 represented by GYSITSDYAWN (SEQ ID NO: 21), GYISNSGSTSYNPSLKSR (SEQ ID NO: 22) and ERNYDYDDYYYAMDY (SEQ ID NO: 23), respectively,
wherein one or more of said CDR sequences may optionally be modified by substitution, addition and/or deletion of 1 to 3 amino acids. The CDR sequences of SEQ ID NOs: 18-23 are derived from an anti-STEAP1 antibody known as Oslo-1.

In an embodiment the CDR sequences are modified by substitution of 1-3 amino acids.

Preferably, any substitution of an amino acid within a CDR sequence is a conservative amino acid substitution. The term “conservative amino acid substitution”, as used herein, refers to an amino acid substitution in which one amino acid residue is replaced with another amino acid residue having a similar side chain. Amino acids with similar side chains tend to have similar properties, and thus a conservative substitution of an amino acid important for the structure or function of a polypeptide may be expected to affect polypeptide structure/function less than a non-conservative amino acid substitution at the same position. Families of amino acid residues having similar side chains have been defined in the art, including amino acids with basic side chains (e.g. lysine, arginine, histidine), acidic side chains (e.g. aspartic acid, glutamic acid), uncharged polar side chains (e.g. asparagine, glutamine, serine, threonine, tyrosine), non-polar side chains (e.g. glycine, cysteine, alanine, valine, leucine, isoleucine, proline, phenylalanine, methionine, tryptophan) and aromatic side chains (e.g. tyrosine, phenylalanine, tryptophan, histidine). Thus, a conservative amino acid substitution may be considered to be a substitution in which a particular amino acid residue is substituted for a different amino acid residue in the same family.

In one embodiment, the antigen-binding domain (e.g. scFv) comprises:
a V_{L} comprising CDR1, CDR2 and CDR3 having the amino acid sequences of SEQ ID NOs: 18, 19 and 20, respectively;
and
a V_{H} comprising CDR1, CDR2 and CDR3 having the amino acid sequences of SEQ ID NOs: 21, 22 and 23, respectively. Thus, in a preferred embodiment the antigen-binding domain comprises the unmodified Oslo-1 CDR sequences.

The Oslo-1 antibody is a known antibody (see e.g. WO 2008/052187 and WO 2018/184966). WO 2018/184966 discloses a number of variants of the Oslo-1 antibody, comprising up to 2 amino acid substitutions in the V_{H}CDR3 sequence relative to the native sequence (SEQ ID NO: 23). In a particular embodiment of the invention, the V_{H}CDR3 sequence comprises up to two amino acid substitutions relative to the native V_{H}CDR3 sequence of SEQ ID NO: 23, and all other CDR sequences are unmodified relative to the native sequences. In other words, CDRs 1 and 2 of the VA region have the amino acid sequences of SEQ ID NOs: 21 and 22 respectively; CDRs 1, 2 and 3 of the Vt region have the amino acid sequences of SEQ ID NOs: 18, 19 and 20, respectively; and CDR3 of the V_{H} region has the amino acid sequence of SEQ ID NO: 23, or an amino acid sequence comprising up to 2 amino acid substitutions relative to SEQ ID NO: 23.

The variant V_{H}CDR3 sequences disclosed in WO 2018/184966 have the amino acid sequences set forth in SEQ ID NOs: 24, 25 and 26. SEQ ID NO: 24 contains an Asp=>Glu substitution at position 7, relative to SEQ ID NO: 23; SEQ ID NO: 25 contains an Asp=>Glu substitution at position 8, relative to SEQ ID NO: 23; SEQ ID NO: 26 contains two Asp=>Glu substitutions at positions 7 and 8 relative to SEQ ID NO: 23. These V_{H}CDR3 sequences may be used in the antigen-binding domain of the CAR. Thus, in a particular embodiment, CDRs 1 and 2 of the V_{H} region have the amino acid sequences of SEQ ID NOs: 21 and 22 respectively; CDRs 1, 2 and 3 of the V_{L} region have the amino acid sequences of SEQ ID NOs: 18, 19 and 20 respectively; and CDR3 of the VA sequence has the amino acid sequence of SEQ ID NO: 24, SEQ ID NO: 25 or SEQ ID NO: 26.

In one embodiment, the antigen-binding domain comprises a V_{H} comprising the amino acid sequence set forth in SEQ ID NO: 27, or a sequence with at least 80, 85, 90 or 95% sequence identity to SEQ ID NO: 27; and a V_{L} comprising the amino acid sequence set forth in SEQ ID NO: 28, or a sequence with at least 80, 85, 90 or 95% sequence identity to SEQ ID NO 28. In particular, it is preferred that the antigen-binding domain is a scFv comprising the aforementioned V_{H} and Vt sequences. Variation relative to the amino acid sequences of SEQ ID NOs: 27 and 28 may be in the form of amino acid substitutions, insertions or deletions, but preferably is in the form of conservative substitution of amino acid residues. While variation of the V_{H} and V_{L} sequences is thus permitted, this is of course subject to the proviso that the CDR sequences remain as defined above, and in one particular embodiment to the proviso that the CDR sequences are unaltered.

The framework regions of the V_{L} and Va sequences may accordingly be modified by one or more amino acid substitutions, additions or deletions. Substitutions of amino acid residues may be tolerated better than deletions or additions of amino acid residues. The substitutions may be conservative substitutions as discussed above.

In a preferred embodiment, the scFv comprises the V_{H} and V_{L} sequences described above, joined by the linker of SEQ ID NO: 17. The CAR may thus comprise an scFv comprising or consisting of the amino acid sequence set forth in SEQ ID NO: 29, or an amino acid sequence having at least 90 or 95% sequence identity thereto. The scFv comprises the V_{H} of SEQ ID NO: 27 at its N-terminus and the V_{L} of SEQ ID NO: 28 at its C-terminus, separated by the linker of SEQ ID NO: 17.

The CAR generally comprises a hinge domain. The hinge domain connects the antigen-binding domain (e.g. scFv) to the transmembrane domain. In particular, the CARs in the present disclosure may comprise a human CD8a hinge (SEQ ID NO: 30) or an IgG-based hinge, e.g. a native IgG hinge or a variant thereof, for example a deletion variant of an IgG hinge comprising a deletion in the hinge sequence. Preferred IgG-based hinges include the native IgG hinge (from human IgG1), which has the sequence set forth in SEQ ID NO: 31, and the IgG-based hinge disclosed in Hombach et al., Gene Therapy 17:1206-1213 (2010), known as the IgGA hinge, which has the amino acid sequence set forth in SEQ ID NO: 32. The CAR may thus comprise the CD8a hinge domain, comprising or consisting of the amino acid sequence of SEQ ID NO: 30, or an amino acid sequence having at least 95% sequence identity thereto; the IgG hinge domain, comprising or consisting of the amino acid sequence of SEQ ID NO: 31, or an amino acid sequence having at least 95% sequence identity thereto; or the IgGA hinge domain, comprising or consisting of the amino acid sequence of SEQ ID NO: 32, or an amino acid sequence having at least 95% sequence identity thereto. Other variants of the native IgG hinge may also be used. In particular, IgG hinge variants comprising one or more, but not all, of the sequence modifications present in the IgGA hinge relative to the native IgG hinge may be used. Other suitable hinge domains include the hinge domains of human CD4, CD28 and CD7.

In particular, the CARs in the present disclosure may comprise an scFv as mentioned above connected to a CD8 hinge, wherein the CAR further comprises a transmembrane domain and wherein the intracellular signalling domain comprises or consists of one costimulatory domain from CD28, 4-1BB or OX40 and a CD3ζ signalling domain.

Alternatively, the CAR in the present disclosure may comprise an scFv as mentioned above connected to an IgG hinge, wherein the CAR further comprises a transmembrane domain and wherein the intracellular signalling domain comprises or consists of one costimulatory domain from CD28, 4-1BB or OX40 and a CD32 signalling domain.

The hinge domain may affect the steric conformation of the scFv. This may in turn affect the ability of the CAR to bind the target epitope and subsequently trigger signalling in an immune cell. If the target epitope is located too far from the cell membrane of the target cell or if the target epitope is otherwise hidden, the immune cell expressing the CAR may not be efficient. Accordingly, it is preferred that the target epitope is sufficiently accessible for immune cells expressing the CARs. It has been found that an IgGA hinge (represented by SEQ ID NO: 32) in the CARs disclosed herein allows the antigen binding domain to bind to the target epitope and allows activation of the immune cell expressing it. Other IgG hinges may work as well provided they allow a similar steric conformation of the antigen binding domain. In particular, such hinges may comprise approximately the same number of amino acid residues as the IgGA hinge represented by SEQ ID NO: 32. It is preferred that the IgG hinge is modified to reduce its affinity for Fc-receptors. This may avoid off-target stimulation of the CAR T cells. One example of such a modified hinge is represented by SEQ ID NO: 32. In particular, the IgGA hinge can be represented by SEQ ID NO: 32 or sequences with at least 90% sequence identity thereto.

It has been found that a human CD8a hinge (represented by SEQ ID NO: 30) in the CARs disclosed herein allows the antigen binding domain to bind to the target epitope and allows activation of the immune cell expressing it. Other CD8a hinges may work as well provided they allow a similar steric conformation of the antigen binding domain. In particular, such hinges may comprise approximately the same number of amino acid residues as the CD8a hinge represented by SEQ ID NO: 30. In particular, the CD8a hinge can be represented by SEQ ID NO: 30 or sequences with at least 90% sequence identity thereto.

Sequence identity may be assessed by any convenient method. However, for determining the degree of sequence identity between sequences, computer programmes that make pairwise or multiple alignments of sequences are useful, for instance EMBOSS Needle or EMBOSS stretcher (both Rice, P. et al., Trends Genet., 16, (6) pp 276-277, 2000) may be used for pairwise sequence alignments while Clustal Omega (Sievers F et al., Mol. Syst. Biol. 7:539, 2011) or MUSCLE (Edgar, R. C., Nucleic Acids Res. 32(5): 1792-1797, 2004) may be used for multiple sequence alignments, though any other appropriate programme may be used. Another suitable alignment programme is BLAST, using the blastp algorithm for protein alignments and the blastn algorithm for nucleic acid alignments. Whether the alignment is pairwise or multiple, it must be performed globally (i.e. across the entirety of the reference sequence) rather than locally.

Sequence alignments and % identity calculations may be determined using for instance standard Clustal Omega parameters: matrix Gonnet, gap opening penalty 6, gap extension penalty 1. Alternatively, the standard EMBOSS Needle parameters may be used: matrix BLOSUM62, gap opening penalty 10, gap extension penalty 0.5. Any other suitable parameters may alternatively be used.

The CAR comprises a transmembrane domain. The transmembrane domain connects the extracellular domains to an intracellular signalling domain. The antigen-binding domain (e.g. scFv) and hinge are the extracellular domains. As used herein, “transmembrane domain”, means the part of the CAR which is embedded in the cell membrane when expressed by an immune effector cell. Suitable transmembrane domains are well-known for the skilled person.

The transmembrane domain may be based on or derived from the transmembrane domain of any transmembrane protein. Typically it may be, or may be derived from, a transmembrane domain from CD8α, CD28, CD4, CD3ζ, CD45, CD9, CD16, CD22, CD33, CD64, CD80, CD86, CD134, CD137, or CD154, preferably from a human version of these proteins. In one embodiment, the transmembrane domain may be, or may be derived from, a transmembrane domain from CD8α, CD28, CD4, or CD3ζ, preferably from human CD8α, CD28, CD4, or CD3ζ. In another embodiment the transmembrane domain may be synthetic in which case it would comprise predominantly hydrophobic residues such as leucine and valine.

In particular, transmembrane domains from CD8a and CD28 can be used in the CAR. The transmembrane domain is believed to convey a signal into immune cells upon binding of a target by the antigen binding domain. It has been found that the CD28 transmembrane domain represented by SEQ ID NO: 33 allows signalling into an immune cell upon binding of a target. It has also been found that the CD8a transmembrane domain represented by SEQ ID NO: 34 allows signalling into an immune cell upon binding of a target. Thus, in a particular embodiment, the transmembrane domain of the CAR is the CD28 transmembrane domain, comprising or consisting of the amino acid sequence of SEQ ID NO: 33, or an amino acid sequence having at least 95% sequence identity thereto. In another embodiment, the transmembrane domain of the CAR is the CD8a transmembrane domain, comprising or consisting of the amino acid sequence of SEQ ID NO: 34, or an amino acid sequence having at least 95% sequence identity thereto.

In a particular embodiment, the CAR comprises an IgG hinge and a CD28 transmembrane domain. In another embodiment, the CAR comprises a CD8α hinge and a CD8α transmembrane domain.

The CAR also comprises an intracellular signalling domain. The “intracellular signalling domain” refers to the part of the CAR located inside the immune cell when the CAR is expressed in the cell membrane. These domains participate in conveying the signal upon binding of the target. In particular, the intracellular signalling domain refers to the part of the CAR protein that participates in transducing the message of effective CAR binding to a target antigen into the interior of the immune effector cell to elicit effector cell function, e.g. activation, cytokine production, proliferation and/or cytotoxic activity, including the release of cytotoxic factors to the CAR-bound target cell, or other cellular responses elicited with antigen binding to the extracellular CAR domain. The term “effector function” refers to a specialized function of the cell. Effector function of the T-cell, for example, may be cytolytic activity or help or activity including the secretion of a cytokine. Thus, the term “intracellular signalling domain” refers to the portion of a protein which transduces the effector function signal and that directs the cell to perform a specialized function.

While an entire intracellular signalling domain can be employed, in many cases it is not necessary to use the entire domain. To the extent that a truncated portion of an intracellular signalling domain is used, such a truncated portion may be used in place of the entire domain as long as it transduces the effector function signal. The term intracellular signalling domain is meant to include any truncated portion of the intracellular signalling domain sufficient to transduce effector function signal. The intracellular signalling domain is also known as the “signal transduction domain”. A variety of signalling domains are known, and they can be combined and tailored to fit the endogenous signalling machinery in the immune cells.

In one embodiment the intracellular signalling domain comprises a “signal 1” domain, such as the signalling domain from CD3ζ, FcR-γ, CD3ε etc. In general, it is believed that “signal 1” domains (e.g. the CD3ζ signalling domain represented by SEQ ID NO: 35) convey a signal upon antigen binding. It is particularly preferred that the CAR comprises the CD3ζ signalling domain, comprising or consisting of the amino acid sequence of SEQ ID NO: 35, or an amino acid sequence having at least 95% sequence identity thereto.

In another embodiment, the intracellular signalling domain further comprises a costimulatory domain. Such domains are well known and often referred to as “signal 2” domains, and they are believed to, subsequently to “signal 1” domains, convey a signal via costimulatory molecules. The “signal 2” is important for the maintenance of the signal and the survival of the cells. If absent, like in first generation CARs, the redirected cell may be efficient in killing and in early cytokine release, but will often become exhausted over time.

The term “co-stimulatory signalling domain” or “co-stimulatory domain”, refers to the portion of the CAR comprising the intracellular domain of a co-stimulatory molecule. Co-stimulatory molecules are cell surface molecules other than antigen receptors or Fc receptors that provide a second signal required for efficient activation and function of an immune effector cell (e.g. a T-cell) upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83, more particularly the intracellular domains of such molecules. Preferably the molecules are human. Accordingly, while exemplary or preferred co-stimulatory domains are derived from 4-1BB, CD28 or OX40 (CD134), other co-stimulatory domains are contemplated for use with the CARs described herein. The co-stimulatory domains may be used singly or in combination (i.e. one or more co-stimulatory domains may be included in a single CAR. The inclusion of one or more co-stimulatory signalling domains may enhance the efficacy and expansion of immune effector cells expressing the CARs. The intracellular signalling and co-stimulatory signalling domains may be linked in any order in tandem to the carboxyl terminus of the transmembrane domain.

As noted above, preferred examples of such “signal 2”, or co-stimulatory, domains include the 4-1BB costimulatory domain (SEQ ID NO: 36) and the CD28 costimulatory domain (SEQ ID NO: 37).

In particular, it has been found that cytotoxic immune cells expressing the CARs herein provide an in vivo effect in prostate cancer models when the intracellular signalling domain comprises or consists of a costimulatory domain (SEQ ID NO: 36 or SEQ ID NO: 37) and a CD3ζ signalling domain (SEQ ID NO: 35). It is thus preferred that the intracellular signalling domain comprises the CD3ζ signalling domain (i.e. the amino acid sequence of SEQ ID NO: 35, or an amino acid sequence having at least 95% sequence identity thereto) and a co-stimulatory domain. In a particular embodiment, the intracellular signalling domain comprises the CD37 signalling domain (i.e. the amino acid sequence of SEQ ID NO: 35, or an amino acid sequence having at least 95% sequence identity thereto) and the 4-1BB co-stimulatory domain, comprising or consisting of the amino acid sequence of SEQ ID NO: 36, or an amino acid sequence having at least 95% sequence identity thereto. In another embodiment, the intracellular signalling domain comprises the CD3ζ signalling domain (i.e. the amino acid sequence of SEQ ID NO: 35, or an amino acid sequence having at least 95% sequence identity thereto) and the CD28 costimulatory domain, comprising or consisting of the amino acid sequence of SEQ ID NO: 37, or an amino acid sequence having at least 95% sequence identity thereto.

For efficient expression of the CAR in immune cells, a conventional leader peptide (also known as signal peptide or L-chain) may be provided to drive localisation of the CAR to the cell membrane. Such a sequence will generally be provided at the N-terminal end of the molecule (construct) and may function to, co-translationally or post-translationally, direct transfer of the molecule to the membrane (i.e. the plasma membrane of an immune effector cell). The signal sequence may be linked directly or indirectly (e.g. via a linker sequence) to the antigen-binding domain of the CAR molecule/construct. Preferably, the signal sequence is linked directly to the N-terminus of the antigen-binding domain. The leader peptide is believed to be trimmed off following delivery of the CAR to the membrane, and so will likely not be present in the functional CAR in the cell membrane. It has been found that the nucleic acids encoding the CARs herein are successfully transcribed and the CARs are successfully localised to the cell membrane when the leader peptide is represented by SEQ ID NO: 38. Thus, in an embodiment, the CAR, as encoded or translated, comprises an N-terminal signal sequence. In a particular embodiment, the N-terminal signal sequence comprises or consists of SEQ ID NO: 38. Alternatively expressed, the CAR encoding sequence of the polycistronic nucleic acid molecule may comprise a nucleotide sequence encoding a leader peptide as described herein.

A number of functional anti-STEAP1 CARs have been developed by the inventors. One of these is the CAR JK10. JK10 comprises, from N-terminus to C-terminus, the scFv of SEQ ID NO: 29, the IgGΔ hinge of SEQ ID NO: 32, the CD28 transmembrane domain of SEQ ID NO: 33, the CD28 costimulatory domain of SEQ ID NO: 37 and the CD3ζ signalling domain of SEQ ID NO: 35. JK10 has the amino acid sequence set forth in SEQ ID NO: 39. In an embodiment of the invention, the CAR is the JK10 CAR or a variant thereof. That is to say, the CAR comprises the amino acid sequence of SEQ ID NO: 39, or an amino acid sequence having at least 95% sequence identity thereto.

Another anti-STEAP1 CAR shown is the CAR JK11. JK11 comprises, from N-terminus to C-terminus, the scFv of SEQ ID NO: 29, the CD8α hinge of SEQ ID NO: 30, the CD8α transmembrane domain of SEQ ID NO: 34, the 4-1BB co-stimulatory domain of SEQ ID NO: 36 and the CD3ζ signalling domain of SEQ ID NO: 35. JK11 has the amino acid sequence set forth in SEQ ID NO: 40. In an embodiment of the invention, the CAR is the JK11 CAR or a variant thereof. That is to say, the CAR comprises the amino acid sequence of SEQ ID NO: 40, or an amino acid sequence having at least 95% sequence identity thereto.

Another anti-STEAP1 CAR is the CAR JK15. JK15 comprises, from N-terminus to C-terminus, the scFv of SEQ ID NO: 29, the IgGΔ hinge of SEQ ID NO: 32, the CD28 transmembrane domain of SEQ ID NO: 33, the 4-1BB co-stimulatory domain of SEQ ID NO: 36 and the CD30 signalling domain of SEQ ID NO: 35. JK15 has the amino acid sequence set forth in SEQ ID NO: 41. In an embodiment of the invention, the CAR is the JK15 CAR or a variant thereof. That is to say, the CAR comprises the amino acid sequence of SEQ ID NO: 41, or an amino acid sequence having at least 95% sequence identity thereto.

Another anti-STEAP1 CAR is the CAR JK16. JK16 comprises, from N-terminus to C-terminus, the scFv of SEQ ID NO: 29, the CD8α hinge of SEQ ID NO: 30, the CD8α transmembrane domain of SEQ ID NO: 34, the CD28 costimulatory domain of SEQ ID NO: 37 and the CD3ζ signalling domain of SEQ ID NO: 35. JK16 has the amino acid sequence set forth in SEQ ID NO: 42. In an embodiment of the invention, the CAR is the JK16 CAR or a variant thereof. That is to say, the CAR comprises the amino acid sequence of SEQ ID NO: 42, or an amino acid sequence having at least 95% sequence identity thereto.

CAR T-cells expressing JK10, JK11, JK15 or JK16 all provided cytotoxic activity against LNCap cells in a caspase assay.

As noted above, the CARs disclosed herein are generally encoded with a leader peptide (signal peptide) in order to target them to the plasma membrane of the immune effector cell in which they are expressed. As detailed above, a suitable leader sequence for inclusion in the CARs of the invention is that of SEQ ID NO: 38. The JK10 CAR comprising the leader sequence of SEQ ID NO: 38 has the amino acid sequence set forth in SEQ ID NO: 43, the JK11 CAR comprising the leader sequence of SEQ ID NO: 38 has the amino acid sequence set forth in SEQ ID NO: 44, the JK15 CAR comprising the leader sequence of SEQ ID NO: 38 has the amino acid sequence set forth in SEQ ID NO: 45 and the JK16 CAR comprising the leader sequence of SEQ ID NO: 38 has the amino acid sequence set forth in SEQ ID NO: 46. In particular embodiments of the invention, the CAR encoding sequence in the polycistronic nucleic acid molecule encodes a CAR comprising or consisting of the amino acid sequence set forth in SEQ ID NO: 43, 44, 45 or 46 (preferably SEQ ID NO: 44 or 45, e.g. SEQ ID NO: 44), or an amino acid sequence having at least 95% sequence identity thereto.

Thus, in a particular embodiment, the polycistronic nucleic acid encodes: (i) a multichain chimeric receptor and; (ii) a chimeric antigen receptor (CAR), wherein:
(A) the multichain chimeric receptor comprises:
(a) a first polypeptide comprising: (i) an extracellular domain comprising the extracellular domain of TGFβRI having an amino acid sequence as set forth in SEQ ID NO: 8 or a functional variant thereof having an amino acid sequence with at least 80% (e.g. at least 85%, 90%, 95%, 96%, 97%, 98% or 99%) sequence identity to an amino acid sequence as set forth in SEQ ID NO: 8; (ii) a transmembrane domain comprising or consisting of the transmembrane domain of IL-2Rβ having an amino acid sequence as set forth in SEQ ID NO: 12 or a functional variant thereof having an amino acid sequence with at least 80% (e.g. at least 85%, 90%, 95%, 96%, 97%, 98% or 99%) sequence identity to an amino acid sequence as set forth in SEQ ID NO: 12; and (iii) an endodomain comprising an intracellular signalling domain of IL-2Rβ having an amino acid sequence as set forth in SEQ ID NO: 10 or a functional variant thereof having an amino acid sequence with at least 80% (e.g. at least 85%, 90%, 95%, 96%, 97%, 98% or 99%) sequence identity to an amino acid sequence as set forth in SEQ ID NO: 10; and
(b) a second polypeptide comprising: (i) an extracellular domain comprising the extracellular domain of TGFβRII having an amino acid sequence as set forth in SEQ ID NO: 9 or a functional variant thereof having an amino acid sequence with at least 80% (e.g. at least 85%, 90%, 95%, 96%, 97%, 98% or 99%) sequence identity to an amino acid sequence as set forth in SEQ ID NO: 9; (ii) a transmembrane domain comprising or consisting of the transmembrane domain of IL-2Rγ having an amino acid sequence as set forth in SEQ ID NO: 13 or a functional variant thereof having an amino acid sequence with at least 80% (e.g. at least 85%, 90%, 95%, 96%, 97%, 98% or 99%) sequence identity to an amino acid sequence as set forth in SEQ ID NO: 13; and (iii) an endodomain comprising an intracellular signalling domain of IL-2Rγ having an amino acid sequence as set forth in SEQ ID NO: 11 or a functional variant thereof having an amino acid sequence with at least 80% (e.g. at least 85%, 90%, 95%, 96%, 97%, 98% or 99%) sequence identity to an amino acid sequence as set forth in SEQ ID NO: 11; and
(B) the CAR comprises N-terminus to C-terminus:
(i) the leader peptide of SEQ ID NO: 38; (ii) the scFv of SEQ ID NO: 29; (iii) the CD8α hinge of SEQ ID NO: 30; (iv) the CD8α transmembrane domain of SEQ ID NO: 34; (v) the 4-1BB co-stimulatory domain of SEQ ID NO: 36; and (vi) the CD37 signalling domain of SEQ ID NO: 35; or
the amino acid sequence of SEQ ID NO: 44 or 45, or an amino acid sequence having at least 95% sequence identity thereto.

In a particularly preferred embodiment, the first polypeptide comprises an amino acid sequence as set forth in SEQ ID NO: 14 or functional variant thereof comprising an amino acid sequence with at least 80% sequence identity to an amino acid sequence as set forth in SEQ ID NO: 14, wherein the functional variant is capable of binding to TGFβ and transducing a IL-2 function signal when expressed in a suitable cell, e.g. an immune cell, such as a T cell, with a functional second polypeptide of the multichain receptor.

In a further particularly preferred embodiment, the second polypeptide comprises an amino acid sequence as set forth in SEQ ID NO: 15 or functional variant thereof comprising an amino acid sequence with at least 80% sequence identity to an amino acid sequence as set forth in SEQ ID NO: 15, wherein the functional variant is capable of binding to TGFβ and transducing a IL-2 function signal when expressed in a suitable cell, e.g. an immune cell, such as a T cell, with a functional first polypeptide of the multichain receptor.

In a still further particularly preferred embodiment, the CAR comprises an amino acid sequence as set forth in SEQ ID NO: 44 or 45 or functional variant thereof comprising an amino acid sequence with at least 95% sequence identity to an amino acid sequence as set forth in SEQ ID NO: 44 or 45, wherein the functional variant is capable of binding to STEAP-1 and transducing a T cell activating signal when expressed in a suitable cell, e.g. an immune cell, such as a T cell.

In a further embodiment, the polycistronic nucleic acid encodes a T2A self-cleaving peptide (or functional variant thereof) between the first and second polypeptides and/or a P2A self-cleaving peptide (or functional variant thereof) between the second polypeptide and the CAR.

Thus, in a further particular embodiment, the polycistronic nucleic acid encodes: (i) a multichain chimeric receptor and; (ii) a chimeric antigen receptor (CAR), wherein the nucleic acid encodes an amino acid sequence as set forth in SEQ ID NO: 47 or 50 or a functional variant thereof comprising an amino acid sequence with at least 95% sequence identity to an amino acid sequence as set forth in SEQ ID NO: 47 or 50.

Thus, in a further particular embodiment, the polycistronic nucleic acid encodes: (i) a multichain chimeric receptor and; (II) a chimeric antigen receptor (CAR) as defined herein, wherein the nucleic acid comprises a nucleotide sequence as set forth in SEQ ID NO: 48 or 51 or a nucleotide sequence with at least 95% sequence identity to a nucleotide sequence as set forth in SEQ ID NO: 48 or 51.

An anti-HER2 CAR is the CAR JK06. JK06 has the amino acid sequence set forth in SEQ ID NO: 53. The JK06 CAR comprising the leader sequence of SEQ ID NO: 38 has the amino acid sequence set forth in SEQ ID NO: 54. In an embodiment of the invention, the CAR is the JK06 CAR or a variant thereof. That is to say, the CAR comprises the amino acid sequence of SEQ ID NO: 53 or 54, or an amino acid sequence having at least 95% sequence identity thereto.

In an embodiment of the invention, the CAR encoding sequence in the polycistronic nucleic acid molecule encodes a CAR comprising or consisting of the amino acid sequence set forth in SEQ ID NO: 53 or 54 (preferably SEQ ID NO: 54), or an amino acid sequence having at least 95% sequence identity thereto.

Thus, in a particular embodiment, the polycistronic nucleic acid encodes: (i) a multichain chimeric receptor and; (ii) a chimeric antigen receptor (CAR), wherein:
(A) the multichain chimeric receptor comprises:
(a) a first polypeptide comprising: (i) an extracellular domain comprising the extracellular domain of TGFβRI having an amino acid sequence as set forth in SEQ ID NO: 8 or a functional variant thereof having an amino acid sequence with at least 80% (e.g. at least 85%, 90%, 95%, 96%, 97%, 98% or 99%) sequence identity to an amino acid sequence as set forth in SEQ ID NO: 8; (ii) a transmembrane domain comprising or consisting of the transmembrane domain of IL-2Rβ having an amino acid sequence as set forth in SEQ ID NO: 12 or a functional variant thereof having an amino acid sequence with at least 80% (e.g. at least 85%, 90%, 95%, 96%, 97%, 98% or 99%) sequence identity to an amino acid sequence as set forth in SEQ ID NO: 12; and (iii) an endodomain comprising an intracellular signalling domain of IL-2Rβ having an amino acid sequence as set forth in SEQ ID NO: 10 or a functional variant thereof having an amino acid sequence with at least 80% (e.g. at least 85%, 90%, 95%, 96%, 97%, 98% or 99%) sequence identity to an amino acid sequence as set forth in SEQ ID NO: 10; and
(b) a second polypeptide comprising: (i) an extracellular domain comprising the extracellular domain of TGFβRII having an amino acid sequence as set forth in SEQ ID NO: 9 or a functional variant thereof having an amino acid sequence with at least 80% (e.g. at least 85%, 90%, 95%, 96%, 97%, 98% or 99%) sequence identity to an amino acid sequence as set forth in SEQ ID NO: 9; (ii) a transmembrane domain comprising or consisting of the transmembrane domain of IL-2Rγ having an amino acid sequence as set forth in SEQ ID NO: 13 or a functional variant thereof having an amino acid sequence with at least 80% (e.g. at least 85%, 90%, 95%, 96%, 97%, 98% or 99%) sequence identity to an amino acid sequence as set forth in SEQ ID NO: 13; and (iii) an endodomain comprising an intracellular signalling domain of IL-2Rγ having an amino acid sequence as set forth in SEQ ID NO: 11 or a functional variant thereof having an amino acid sequence with at least 80% (e.g. at least 85%, 90%, 95%, 96%, 97%, 98% or 99%) sequence identity to an amino acid sequence as set forth in SEQ ID NO: 11; and
(B) the CAR comprises the amino acid sequence of SEQ ID NO: 53 or 54, or an amino acid sequence having at least 95% sequence identity thereto.

In a particularly preferred embodiment, the first polypeptide comprises an amino acid sequence as set forth in SEQ ID NO: 14 or functional variant thereof comprising an amino acid sequence with at least 80% sequence identity to an amino acid sequence as set forth in SEQ ID NO: 14, wherein the functional variant is capable of binding to TGFβ and transducing a IL-2 function signal when expressed in a suitable cell, e.g. an immune cell, such as a T cell, with a functional second polypeptide of the multichain receptor.

In a further particularly preferred embodiment, the second polypeptide comprises an amino acid sequence as set forth in SEQ ID NO: 15 or functional variant thereof comprising an amino acid sequence with at least 80% sequence identity to an amino acid sequence as set forth in SEQ ID NO: 15, wherein the functional variant is capable of binding to TGFβ and transducing a IL-2 function signal when expressed in a suitable cell, e.g. an immune cell, such as a T cell, with a functional first polypeptide of the multichain receptor.

In a still further particularly preferred embodiment, the CAR comprises an amino acid sequence as set forth in SEQ ID NO: 53 or 54 or functional variant thereof comprising an amino acid sequence with at least 95% sequence identity to an amino acid sequence as set forth in SEQ ID NO: 53 or 54, wherein the functional variant is capable of binding to HER2 and transducing a T cell activating signal when expressed in a suitable cell, e.g. an immune cell, such as a T cell.

In a further embodiment, the polycistronic nucleic acid encodes a T2A self-cleaving peptide (or functional variant thereof) between the first and second polypeptides and/or a P2A self-cleaving peptide (or functional variant thereof) between the second polypeptide and the CAR.

Thus, in a further particular embodiment, the polycistronic nucleic acid encodes: (i) a multichain chimeric receptor and; (II) a chimeric antigen receptor (CAR), wherein the nucleic acid encodes an amino acid sequence as set forth in SEQ ID NO: 55 or a functional variant thereof comprising an amino acid sequence with at least 95% sequence identity to an amino acid sequence as set forth in SEQ ID NO: 55.

Thus, in a further particular embodiment, the polycistronic nucleic acid encodes: (i) a multichain chimeric receptor and; (ii) a chimeric antigen receptor (CAR) as defined herein, wherein the nucleic acid comprises a nucleotide sequence as set forth in SEQ ID NO: 58 or a nucleotide sequence with at least 95% sequence identity to a nucleotide sequence as set forth in SEQ ID NO: 58.

The terms “polypeptide” and “protein” are used interchangeably and mean a polymer of amino acids not limited to any particular length. The term does not exclude modifications such as myristylation, sulfation, glycosylation, phosphorylation and addition or deletion of signal sequences. The terms “polypeptide” and “protein” mean one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” specifically encompass the multichain chimeric receptors chains and CARs of the present disclosure, or sequences that have deletions from, additions to, and/or substitutions of one or more amino acid of a multichain chimeric receptors chain or CAR as disclosed herein.

As is clear from the above, the various domains of the multichain chimeric receptor and CAR may comprise one or more amino acid sequence modifications with respect to the native sequences of the molecules from which they are derived. For example, it may be desirable to improve the binding affinity and/or other biological properties of the multichain chimeric receptor and/or CAR. For example, amino acid sequence variants of a multichain chimeric receptor, CAR, or binding domain, or a stimulatory signalling domain thereof, may be prepared by introducing appropriate nucleotide changes into a polynucleotide that encodes the multichain chimeric receptor, CAR, or a domain thereof. Such modifications include, for example, deletions from, and/or insertions into and/or substitutions of, residues within the amino acid sequences of the multichain chimeric receptor or CAR. Any combination of deletion, insertion, and substitution may be made to arrive at the final multichain chimeric receptor and/or CAR, provided that the final construct possesses the desired characteristics, such as specific binding to TGFβ by the binding domain of the multichain chimeric receptor, specific binding to STEAP1 by the antigen binding domain of the CAR, or increased signalling by the intracellular signalling domain and/or co-stimulatory domain. The amino acid changes also may alter post-translational processes of the multichain chimeric receptor or CAR, such as changing the number or position of glycosylation sites. Any of the variations and modifications described above may be included in the multichain chimeric receptor and CARs disclosed herein.

The polycistronic nucleic acid molecule of the invention may be an isolated nucleic acid molecule and may further include DNA or RNA or chemical derivatives of DNA or RNA, including molecules having a radioactive isotope or a chemical adduct such as a fluorophore, chromophore or biotin (“label”). Thus, the nucleic acid may comprise modified nucleotides. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “nucleic acid molecule” specifically includes single- and double-stranded forms of DNA and RNA.

Methods for modifying nucleotide sequences to introduce changes to the amino acid sequences of the various domains are well known in the art, e.g. methods of mutagenesis, such as site-specific mutagenesis, may be employed.

Likewise methods for preparing a nucleic acid molecule are also well known e.g. conventional polymerase chain reaction (PCR) cloning techniques can be used to construct the nucleic acid molecule. The nucleic acid molecule can be cloned into a general purpose cloning vector such as pENT (Gateway), pUC19, pBR322, pBluescript vectors (Stratagene Inc.) or pCR TOPO® from Invitrogen Inc. The resultant nucleic acid construct (recombinant vector) carrying the polycistronic nucleic acid molecule can then be sub-cloned into expression vectors or viral vectors for protein expression, e.g. in mammalian cells. This may be for preparation of the proteins encoded by the polycistronic nucleic acid, or for expression in immune effector cells, e.g. in human T-cells or in NK cells or cell lines. Further the nucleic acid may be introduced into mRNA expression vectors for production of polycistronic mRNA encoding the multichain chimeric receptor, CAR and/or TCR. The mRNA may then be transferred into immune effector cells.

Thus, the polycistronic nucleic acid of the invention can be provided in the context of a vector. The vector may be an RNA (e.g. mRNA) or DNA vector (e.g. expression vectors, retroviral vectors etc.). The vector may be a cloning vector or an expression vector. Expression vectors can contain a variety of control sequences, which refer to nucleic acid sequences necessary for the transcription and possibly translation of an operatively linked coding sequence in a particular host cell. In addition to control sequences that govern transcription and translation, vectors may contain additional nucleic acid sequences that serve other functions, including for example for replication, selectable markers etc. Such control sequences, and other functional sequences, are well-known in the art.

For cloning of the nucleic acid molecule the vector may be introduced into a host cell (e.g. an isolated host cell) and such “production host cells” containing a cloning vector of the invention may form a further aspect of the invention. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheniformis, Pseudomonas such as P. aeruginosa, and Streptomyces. A production host cell may alternatively contain an mRNA expression vector comprising the nucleic acid molecule.

The nucleic acid molecules or vectors may be introduced into a host cell (e.g. a production host cell or an immune effector cell) using transfection and/or transduction techniques known in the art. As used herein, the terms, “transfection,” and, “transduction,” refer to the processes by which an exogenous nucleic acid sequence is introduced into a host cell. The nucleic acid may be integrated into the host cell DNA or may be maintained extra-chromosomally. The nucleic acid may be maintained transiently or may be stably introduced. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, lentiviral or retroviral infection, and biolistics. Transduction refers to the delivery of a gene(s) using a viral or retroviral vector by means of viral infection rather than by transfection. In certain embodiments, retroviral vectors are transduced by packaging the vectors into viral particles or virions prior to contact with a cell.

In a particular embodiment, the polycistronic nucleic acid is provided in a viral vector, particularly a retroviral vector, such as a gamma retroviral vector. In a particular embodiment, the retroviral vector comprises a nucleotide sequence as set forth in SEQ ID NO: 49 or 52 or a nucleotide sequence with at least 95% sequence identity to a nucleotide sequence as set forth in SEQ ID NO: 49 or 52.

In some embodiments, the retroviral vector sequence variants encode the same proteins as SEQ ID NO: 49 or 52. For instance, the sequence variants may be further codon-optimised relative to SEQ ID NO: 49 or 52.

A further aspect of the invention is a plurality of viral particles or virions comprising the viral vector described herein.

An “Immune effector cell,” is any cell of the immune system that has one or more effector functions (e.g. cytotoxic cell killing activity, secretion of cytokines, induction of ADCC and/or CDC). Representative immune effector cells thus include T lymphocytes, in particular cytotoxic T cells (CTLs; CD8+ T cells) and helper T-cells (HTLs; CD4+ T cells). Other populations of T cells are also useful herein, for example naive T cells and memory T cells. Other immune effector cells include NK cells, NKT cells, neutrophils, and macrophages. Immune effector cells also include progenitors of effector cells, wherein such progenitor cells can be induced to differentiate into immune effector cells in vivo or in vitro. As described further below, the cells may be primary cells or cell lines.

T cells, particularly CD8+ T cells, and NK cells represent preferred immune effector cells according to the invention. The invention provides an immune effector cell comprising a nucleic acid molecule or vector of the invention and expressing a multichain chimeric receptor and CAR or TCR as described herein at its surface.

The term “NK cell” refers to a large granular lymphocyte, being a cytotoxic lymphocyte derived from the common lymphoid progenitor which does not naturally comprise an antigen-specific receptor (e.g. a T-cell receptor or a B-cell receptor). NK cells may be differentiated by their CD3−, CD56+ phenotype. The term as used herein thus includes any known NK cell or any NK-like cell or any cell having the characteristics of an NK cell. Thus, primary NK cells may be used or in an alternative embodiment, a NK cell known in the art that has previously been isolated and cultured may be used. Thus, an NK cell-line may be used. A number of different NK cells are known and reported in the literature and any of these could be used, or a cell-line may be prepared from a primary NK cell, for example by viral transformation (Vogel et al. 2014, Leukemia 28:192-195). Suitable NK cell lines include (but are by no means limited to), in addition to NK-92, the NK-YS, NK-YT, MOTN-1, NKL, KHYG-1, HANK-1, or NKG cell lines. Similarly, a T cell of the invention may be a primary T cell, or a T cell line. Preferably, the immune effector cell of the invention is a human NK cell or a human T cell (e.g. a human CD8+ T cell or a human CD4+ T cell). Such cells are particularly suitable for treatment of human patients.

The immune cells used to express the polycistronic nucleic acid herein may be isolated from a patient or a compatible donor by leukapheresis or other suitable methods. As noted above, such primary cells may in particular be T cells or NK cells. In particular, autologous T cells (both cytotoxic T cells, T helper cells or mixtures of these) may be transduced with the polycistronic nucleic acid before a pharmaceutical composition comprising the cells is administered to the patient.

The pharmaceutical compositions herein can be any composition suitable for administration of therapeutic cells to a patient. The most common administration route for immune effector cells (e.g. CAR-T cells) is intravenous administration. Accordingly, said pharmaceutical compositions may for example be sterile aqueous solutions with a neutral pH. For example, a patient's peripheral blood mononuclear cells may be obtained via a standard leukapheresis procedure. The mononuclear cells may be enriched for T cells, before transducing them with a viral vector (e.g. lentiviral or retroviral vector) or polycistronic mRNA of the invention. Said cells may then be activated with anti-CD3/CD28 antibodies. In some embodiments, the anti-CD3 antibody and optionally the anti-CD28 antibody may be immobilised antibodies, e.g. plate-bound or bead-bound antibodies. The transduced T cells may be expanded in cell culture, washed, and formulated into a sterile suspension, which can be cryopreserved. If so, the product is thawed prior to administration.

As noted above, the inventors have advantageously determined that expression of the multichain receptor and/or a CAR at the surface of immune effector cells can be increased when the cells transduced with a retroviral vector comprising the polycistronic nucleic acid are frozen (i.e. cryopreserved), thawed and subsequently activated and optionally expanded.

Accordingly, the invention may provide a cell population comprising the immune effector cell disclosed herein, wherein the cell population is frozen (i.e. cryopreserved). Alternatively viewed, the invention may provide a frozen (i.e. cryopreserved) cell population comprising the immune effector cell disclosed herein. The invention may also provide a cell population comprising the immune effector cell disclosed herein wherein the cell population is an activated and optionally expanded cell population obtained from immune effector cells that have been frozen (i.e. cryopreserved) and subsequently thawed.

Furthermore, the invention may be seen to provide a method for increasing the expression of a multichain chimeric receptor as defined herein and/or a CAR or TCR as defined herein at the surface of immune effector cells in a cell population, the method comprising:
(a) providing a cell population comprising the immune effector cell comprising the polycistronic nucleic acid as defined herein or the vector (e.g. retroviral vector) as defined herein;
(b) freezing (i.e. cryopreserving) the cell population of (a);
(c) thawing the frozen cell population of (b); and
(d) activating and optionally expanding the thawed cell population of (c).

Cryopreservation, thawing, activation and expansion of the immune cells may be achieved using any suitable technique known in the art and is well within the purview of the skilled person. Representative examples of cryopreservation, thawing, activation and expansion are set out in the Examples. For instance, cells may be activated using anti-CD3 and anti-CD28 antibodies.

Pharmaceutical compositions of the present invention may thus comprise an immune effector cell population, such as T cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g. aluminium hydroxide); and preservatives.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono- or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

As detailed above, novel polycistronic nucleic acids encoding a multichain chimer receptor and CAR or TCR are provided herein. When the multichain chimeric receptor and CAR or TOR are expressed on the surface of immune cells, such immune cells may be used in medicine/therapy. In particular, said immune cells may be used in the treatment of cancer, particularly solid tumours, such as prostate cancer (e.g. when the CAR is directed to STEAP-1). In one embodiment, said immune cells may be used in the treatment of metastatic prostate cancer. In one embodiment, said immune cells may be used in the treatment of castration-resistant prostate cancer. In another embodiment, said immune cells may be used in the treatment of metastatic castration-resistant prostate cancer.

In contrast to haematological malignancies, recognition of solid tumours requires migration of immune cells from the blood into the tumours. In situations where the tumour is localized, different administration methods may be used to improve efficacy. For example, regional rather than systemic administration of immune cells might enhance efficacy. In other embodiments, systemic administration of the immune cells may be advantageous.

The pharmaceutical compositions may comprise a pharmaceutically effective dose of the immune cells herein. A pharmaceutically effective dose may for example be in the range of 1×10⁶ to 1×10¹⁰ immune cells expressing the multichain chimeric receptor and CAR or TOR. A pharmaceutically effective dose may for example be in the range of 1×10⁶ to 1×10⁹ T cells expressing the multichain chimeric receptor and CAR or TCR.

In one embodiment, a pharmaceutical composition is provided, which comprises a pharmaceutically effective dose of T cells or NK cells expressing the multichain chimeric receptor and CAR or TCR as described herein, for use in treatment of cancer, such as STEAP1-positive cancer. Such compositions can be administered intravenously. STEAP1-positive cancer cells can be detected by anti-STEAP1 antibodies (e.g. X120.545.1.1 from ATCC PTA-5803).

In one particular embodiment, a method of treating a patient diagnosed with cancer (e.g. prostate cancer) is provided, wherein the method comprises the steps:
a. obtaining a biological sample comprising cancer cells from the patient;
b. analysing whether the cancer cells express STEAP1; and
c. administering a pharmaceutical composition comprising a pharmaceutically effective dose of T cells or NK cells expressing the multichain chimeric receptor and CAR disclosed herein if the cancer cells are STEAP1 positive.

The multichain chimeric receptor and CAR/TCR-expressing immune effector cells may be administered in combination with one or more other therapeutic agents, which may include any other known cancer treatments, such as radiation therapy, chemotherapy, transplantation, immunotherapy, hormone therapy, photodynamic therapy, etc. The compositions may also be administered in combination with antibiotics or other therapeutic agents, including e.g. cytokines (e.g. IL-1, IL-2, IL-3, IL-4, IL-5, IL-6, IL-7, IL-8, IL-9, IL-10, IL-11, IL-12, IL-13, IL-15 and IL-17), growth factors, steroids, NSAIDs, DMARDs, anti-inflammatories, analgesics, chemotherapeutics (e.g. monomethyl auristatin E, fludarabine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, oxaliplatin, mitomycin, dacarbazine, procarbazine, etoposide, teniposide, campathecins, bleomycin, doxorubicin, idarubicin, daunorubicin, dactinomycin, plicamycin, mitoxantrone, L-asparaginase, 5-fluorouracil), radiotherapeutics, immune checkpoint inhibitors (e.g. Tremelimumab, Ipilimumab, Nivolumab, MK-3475, Urelumab, Bavituximab, MPDL3280A, MEDI4736), small molecule inhibitors or other active and ancillary agents.

As discussed above, although the multichain chimeric receptor finds particular utility when expressed in combination with a tumour-targeting receptor, such as a CAR or heterologous TCR, particularly in the context of the polycistronic nucleic acid molecule of the invention, it will be appreciated by the skilled person that the multichain chimeric receptor also may be useful in enhancing the activity of other immune cells in an immunosuppressive microenvironment. In particular, it has been shown that some immune cells do not require modification in order to direct them to a tumour target, e.g. tumour-infiltrating lymphocytes (TILs) and natural killer cells (e.g. allogeneic natural killer cells).

TILs are T cells that can be isolated from a tumour and that can recognize and kill cancer cells. In cancer therapy, TILs are removed from a patient's tumour, expanded in vitro, and subsequently administered to the patient to help the immune system kill the cancer cells. In view of the data provided in the Examples below, it is expected that TILs expressing the multichain chimeric receptor disclosed herein, e.g. via transduction with a bicistronic vector encoding the chains separated by a sequence encoding a self-cleaving peptide, will be more effective in cancer therapies.

Similarly, NK cells express an array of germline-encoded surface receptors that enables them to recognize and rapidly act against malignant cells without prior sensitization. In this respect, tumourigenic cells commonly have downregulated MHC I expression but upregulated levels of ligands for NK cell activating receptors and thus trigger NK cell activation due to the lack of inhibitory signals and/or the presence of activating signals. Upon activation, NK cells release cytotoxic granules containing perforin and granzymes to directly lyse tumour cells, in a similar fashion to activated cytotoxic T cells. NK cells also play a significant role in antibody-mediated cancer therapies by utilizing the Fcγ receptor to carry out antibody-dependent cellular cytotoxicity (ADCC).

Thus, similarly to TILs, NKs expressing the multichain chimeric receptor disclosed herein, e.g. via transduction with a bicistronic vector encoding the chains separated by a sequence encoding a self-cleaving peptide, are expected to be more effective in cancer therapies as they will be able to persist and be active for longer in immunosuppressive environments, such as tumour microenvironments. Advantageously, NK cells do not need to be isolated and expanded from the subject to be treated. The NK cells may be allogeneic cells, including cell lines as described above.

Thus, the invention further provides a tumour-infiltrating lymphocyte (TIL) or natural killer (NK) cell (e.g. an allogeneic NK cell) expressing a multichain chimeric receptor as defined herein, i.e. a modified TIL or NK cell.

Furthermore, disclosed herein is the TIL or NK cell (e.g. allogeneic NK cell) as described herein for use in therapy.

Further disclosed herein is the TIL or allogeneic NK cell disclosed herein for use in treating cancer, optionally wherein the cancer is selected from melanoma, non-small cell lung cancer, ovarian cancer, head and neck cancer, colorectal cancer, liver cancer, breast cancer, soft tissue sarcoma, pancreatic cancer, cervical cancer, renal cancer, gastric cancer, osteosarcoma, oesophageal cancer, glioblastoma, thyroid cancer, neuroendocrine cancer, mesothelioma, bladder cancer, prostate cancer, multiple myeloma, small cell lung cancer and endometrial cancer.

Thus, the invention may be seen to provide a method for treating and/or preventing a disease or condition in a subject (e.g. cancer), which comprises the step of administering an engineered TIL or NK cell as defined above, cell population comprising said cell or pharmaceutical composition comprising said cell or cell population to the subject. The method may comprise the step of administering a population of cells to a subject.

The method may involve the following steps:
(i) taking a sample of cells, such as a blood sample or tumour sample from a patient,
(ii) extracting the immune cell, e.g. TIL or NK cell,
(iii) introducing into the cells (e.g. transducing or transfecting the cells) a vector or a nucleic acid encoding the multichain chimeric receptor disclosed herein,
(iv) expanding the cells comprising the nucleic acid or vector (i.e. the modified or engineered cells) ex-vivo, and
(v) returning the cells to the subject.

In some embodiments, steps (i) and (ii) may be viewed as providing a cell-containing sample (e.g. TIL containing sample), particularly obtained from a subject.

It will be evident that for allogeneic NK cells, steps (i) and (ii) may be viewed as providing a NK cell-containing sample, which may be a cell line or a sample obtained from a subject other than the subject to be treated.

The subject to be treated using the methods and cells of the present invention may be any species of mammal. For instance, the subject may be any species of domestic pet, such as a mouse, rat, gerbil, rabbit, guinea pig, hamster, cat or dog, or livestock, such as a goat, sheep, pig, cow or horse. In a preferred embodiment of the invention the subject is a primate, such as a monkey, gibbon, gorilla, orang-utang, chimpanzee or bonobo. However, in a preferred embodiment of the invention the subject is a human. Whilst the use of xenogeneic cells is not precluded, it is preferred that immune cells for administration are of the same species as the recipient. Thus, for human subjects it is preferred that the immune cells are human.

The present invention may be more fully understood from the non-limiting Examples below with reference to the following drawings.

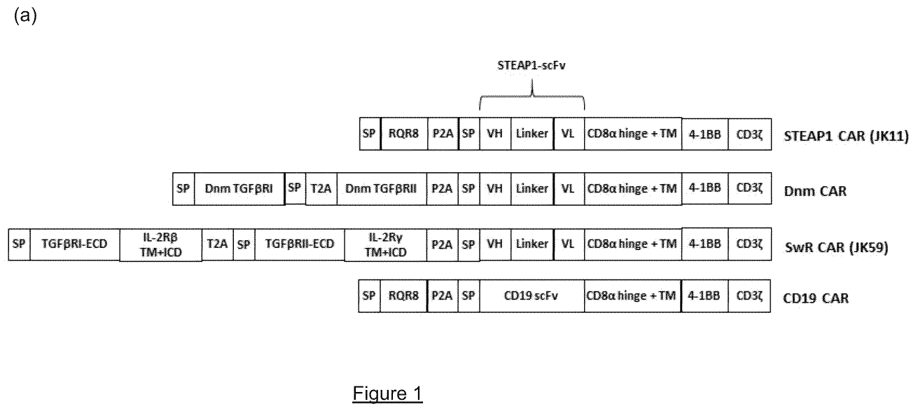

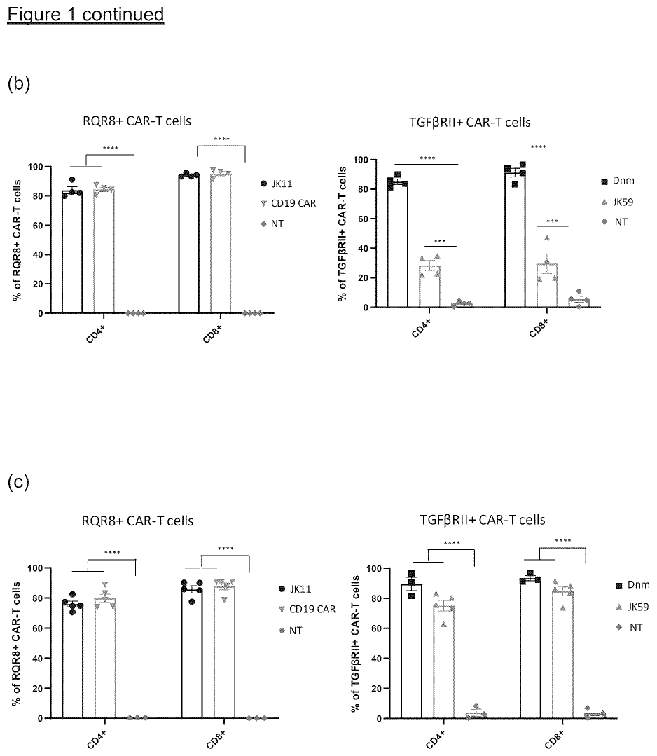

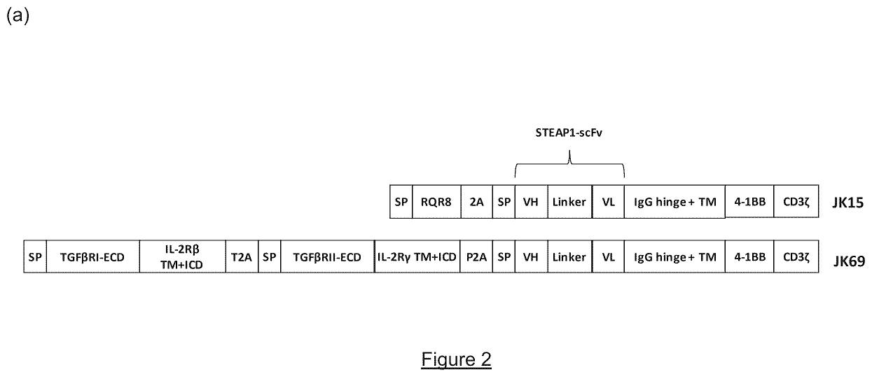

FIG. 1: CAR design and expression: (a) Schematic representations of the CAR constructs: parental STEAP1 CAR (JK11); dominant negative mutant-STEAP1 CAR (Dnm CAR) with the TGFβ receptor (TGFβR) lacking the intracellular signalling domain; TGFβ-switch receptor (SwR)-STEAP1 CAR (JK59) with the extracellular domain (ECD) of the TGFβRI and TGFβRII subunits fused to the transmembrane domain (TM) and intracellular domain (ICD) of the IL-2/IL-15 receptor β and γ chains, respectively (IL-2Rβ and IL-2Rγ); and control CD19 CAR. (b) Graphs representing the expression of JK11 and CD19 CARs (right panel) and Dnm and JK59 CARs (left panel). Data represents the mean values #SEM of 4 different T cell transductions from 4 different healthy donors (each transduction was done in duplicate). (c) Graphs representing the expression of JK11 and CD19 CARs (left panel) and Dnm and JK59 CARs (right panel) after freeze-thawing and activation for two days, from 3 to 5 healthy donors. Data represents the mean values #SEM. Data were analyzed by one-way ANOVA with Tukey's multiple comparisons test. *p<0.001; *p<0.0001.

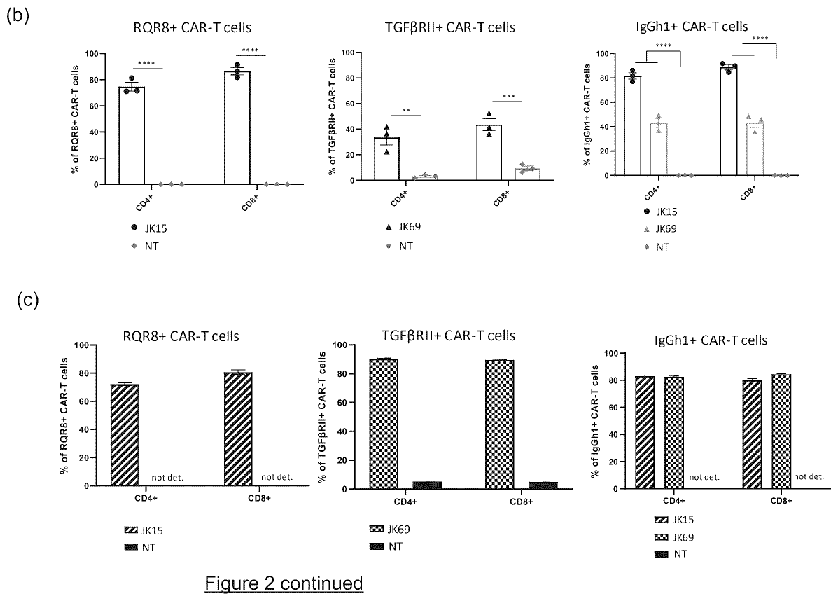

FIG. 2: CAR design and CAR expression of JK15 and JK69: (a) Schematic representation of the STEAP1-IgG (JK15) CAR and the TGFβ-switch receptor (SwR)-IgG (JK69) CAR with the extracellular domain (ECD) of the TGFβRI and TGFβRII subunits fused to the transmembrane domain (TM) and intracellular domain (ICD) of the IL-2/IL-15 receptor β and γ chains, respectively (IL-2Rβ and IL-2Rγ). Both CARs contain the human IgG1 (IgGh1) hinge and TM domain. (b) Graphs representing the expression of JK15 and JK69 CARs. Data represents the mean values±SEM of three different T cell transductions from three different healthy donors. Each transduction was done in duplicate. (c) Graphs representing the expression of JK15 and JK69 CARs after freezing/thawing and activation with anti-CD3/CD28 antibodies for 2 days from one healthy donor. Data represents the mean values #SEM. Data were analyzed by one-way ANOVA with Tukey's multiple comparisons test. **p<0.01; ***p<0.001; ****p<0.0001. not det.: =not detected.

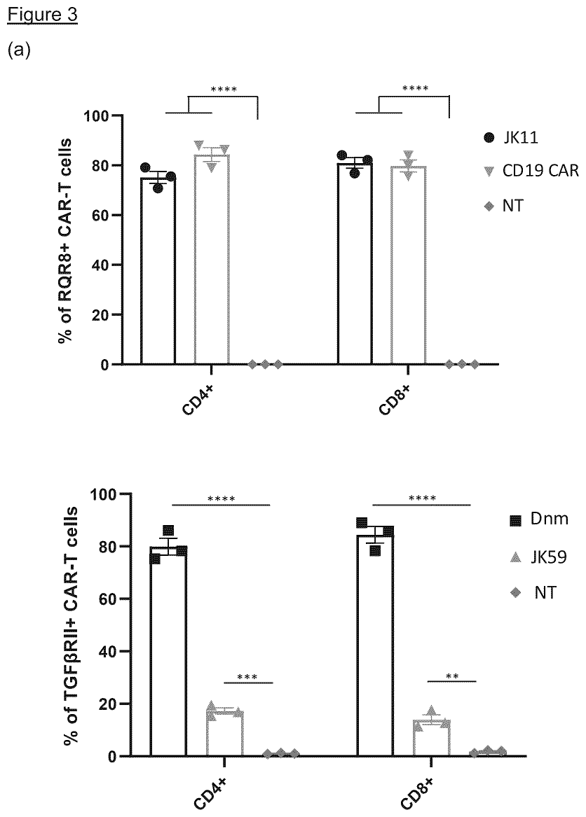

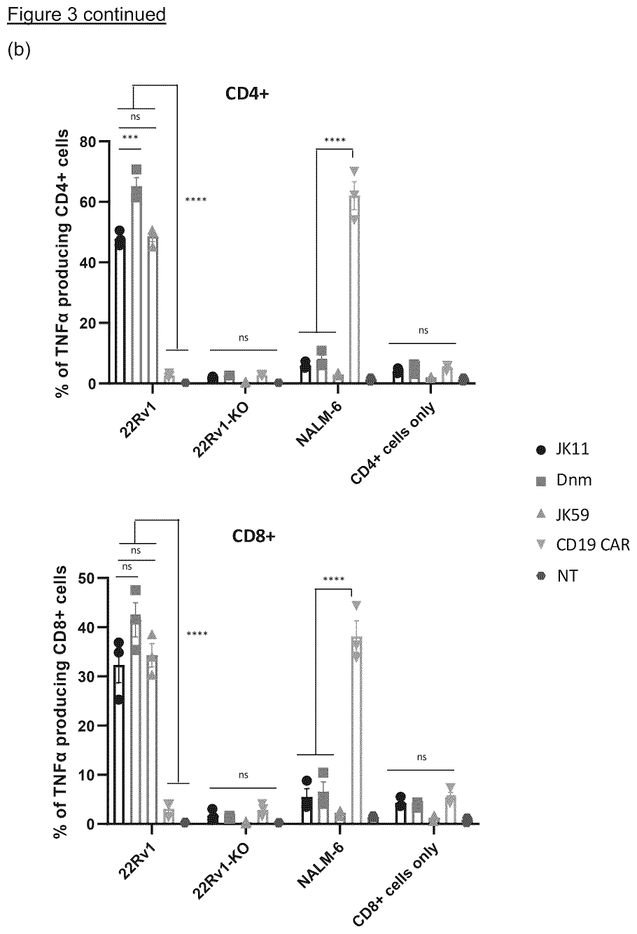

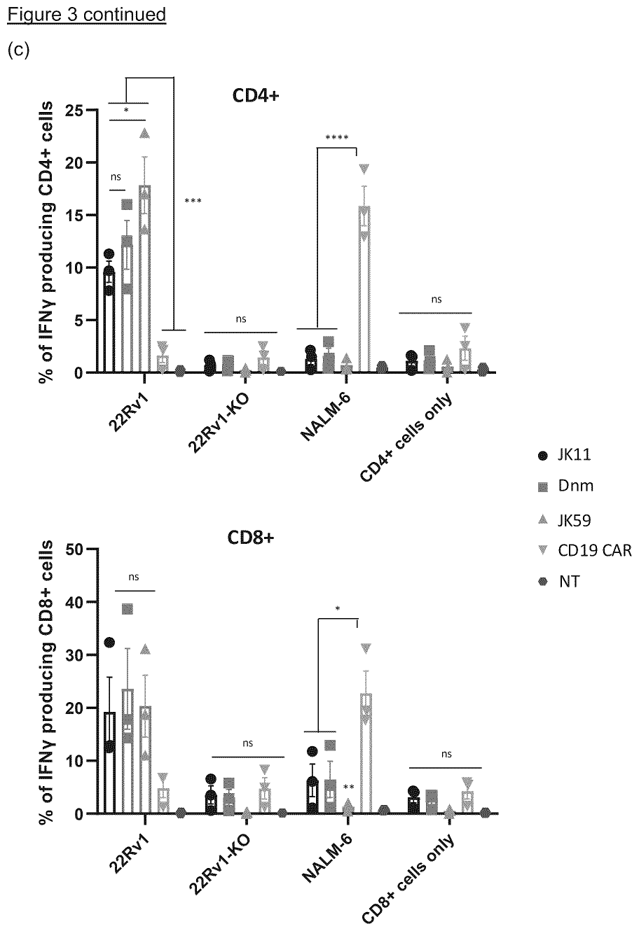

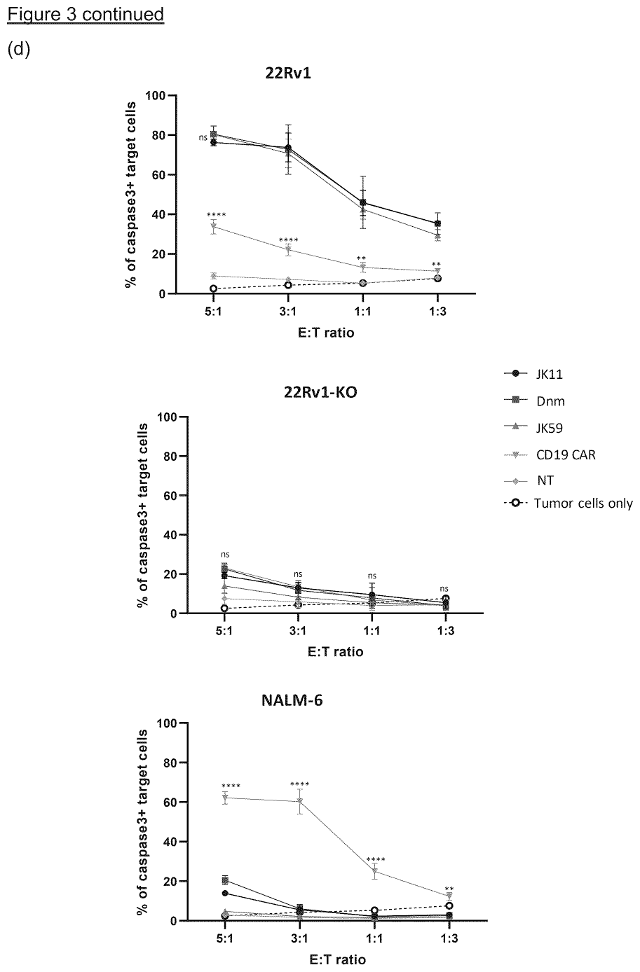

FIG. 3: Dnm and SwR-STEAP1 CAR T cells induce the production of IFNγ and TNFα and apoptosis upon co-culture with STEAP1+ target cells in vitro: (a) Graphs of the CAR expression (measured five days after transduction) of freshly transduced JK11, Dnm, JK59 and CD19 CAR T cells, and non-transduced (NT) T cells. The CAR expression of JK11 and CD19 CAR T cells (top panel) was detected with the antibody recognizing the RQR8 sequence and the CAR expression of Dnm and JK59 CAR T cells (bottom panel) was detected with the TGFβRII antibody. Data represents mean values #SEM of three different healthy donors, each in triplicate. Data were analyzed by two-way ANOVA with Tukey's multiple comparisons test. **<p0.01; ***p<0.001; ****p<0.0001. (b) and (c) CAR T cells and non-transduced (NT) cells were co-cultured (in duplicate) with 22Rv1, 22Rv1-KO and NALM-6 target cells at an ET ratio of 1:3, for 18 hours. Production of TNFα and IFNγ were measured with flow cytometry by gating on CD3+, and CD4+ or CD8+ cells. (b) Percentage of TNFα production in CD4+ (top panel) and CD8+ (bottom panel) T cells. (c) Percentage of IFNγ production in CD4+ (top panel) and CD8+ (bottom panel) T cells. CAR T cells cultured alone (CD4+ and CD8+ cells only) were included as controls to indicate the baseline of TNFα (in (b)) and of IFNγ (in (c)). Data represents mean values #SEM of three different healthy donors, each in duplicate. Graphs are representative of two independent experiments. Data were analyzed by oneway ANOVA with Tukey's multiple comparisons test. *<p0.05; ***p<0.001; ****p<0.0001; ns=not significant. (d) Graphs representing the percentage of apoptotic cells among 22Rv1 (top panel), 22Rv1-KO (middle panel) and NALM-6 (bottom panel) cells co-cultured with the different CAR T cell groups, or the NT control group at E:T ratios 1:3, 1:1, 3:1 and 5:1, for 24 hours. Target cells cultured alone (tumour cells only) were included as controls to indicate the baseline of active caspase3. Data represents mean values #SEM of two different healthy donors, each in duplicate. Graphs are representative of two independent experiments. Data were analyzed by two-way ANOVA with Tukey's multiple comparisons test. **<p0.01; ****p<0.0001; ns=not significant.

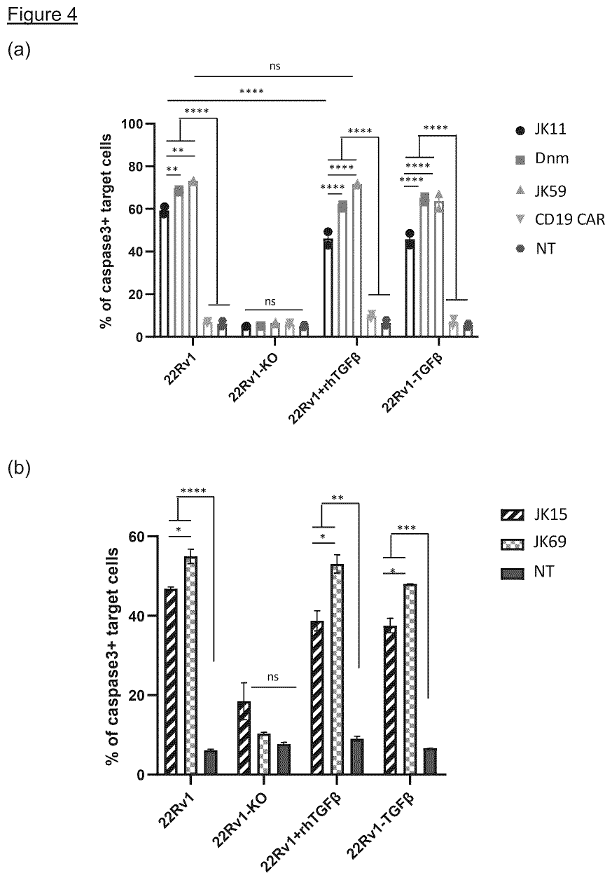

FIG. 4: SwR-STEAP1 CAR T cells has superior cytotoxic activity in TGFβ-rich environment: (a) 22Rv1, 22Rv1-KO, and 22Rv1-TGFβ target cell lines, and 22Rv1 target cells in the presence of 10 ng/ml rhTGFβ were co-cultured with freeze-thawed and non-activated JK11, Dnm, JK59 and CD19 CAR T cells, or NT T cells, at an E:T ratio of 1:1 for 48 hours. A graph representing the percentage of apoptotic cells among 22Rv1, 22Rv1-KO, and 22Rv1-TGFβ cell lines, and 22Rv1 cells cultured in the presence of 10 ng/ml rhTGFβ. Data represents the mean values±SEM of two healthy donors from one representative experiment, in which each co-culture was performed in duplicate. The experiment was repeated two times. Data were analyzed by two-way ANOVA with Tukey's multiple comparisons test. **p<0.01; ****p<0.0001; ns=not significant. (b) 22Rv1, 22Rv1-KO, 22Rv1-TGFβ target cell lines, and 22Rv1 target cells in the presence of 10 ng/ml rhTGFβ were co-cultured with frozen/thawed and non-activated JK15 and JK69 CAR T cells, or non-transduced (NT) T cells, at an E:T ratio of 1:1 for 48 hours. A graph representing the percentage of apoptotic cells among 22Rv1, 22Rv1-KO, 22Rv1-TGFβ cell lines, and 22Rv1 cells cultured in the presence of 10 ng/ml rhTGFβ. Data represents the mean values #SEM of one healthy donor (in duplicates) from one representative experiment. The experiment was repeated two times Data were analyzed by one-way ANOVA with Tukey's multiple comparisons test. *<p0.05; **p<0.01; ***p<0.001; ****p<0.0001; ns=not significant.

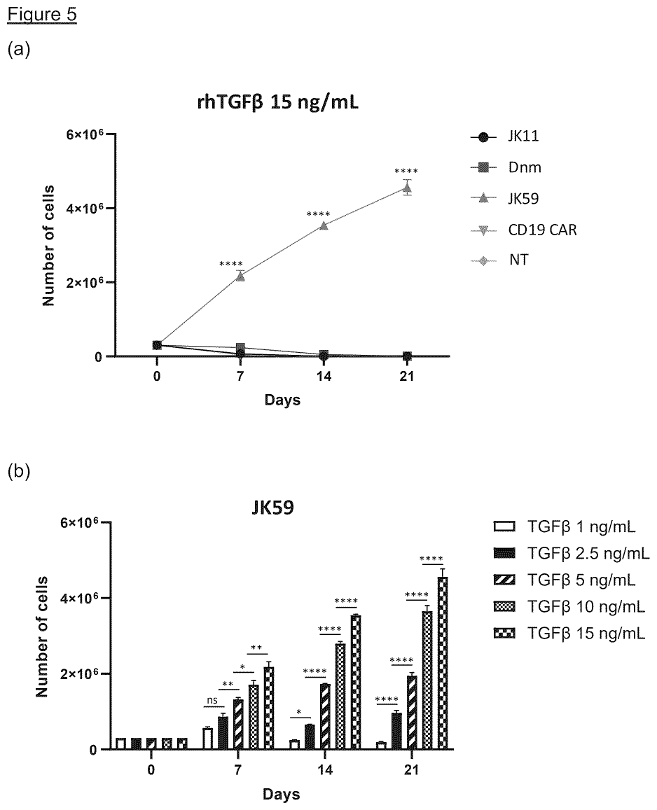

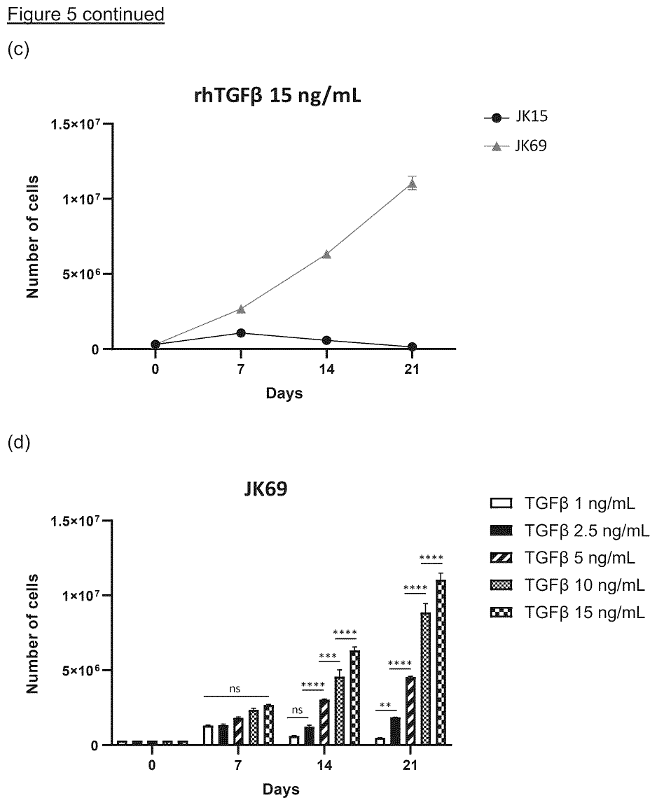

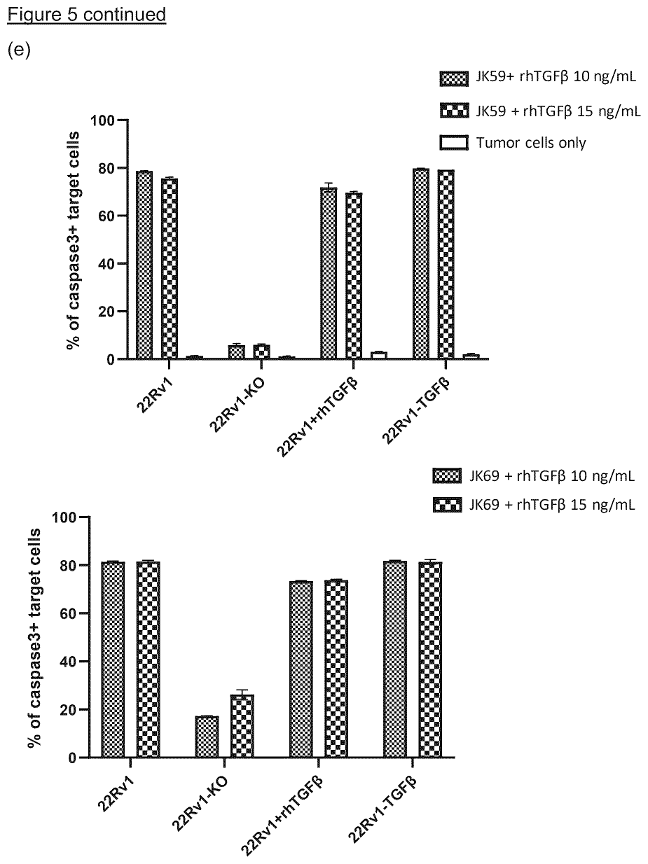

FIG. 5: TGFβ induces proliferation of the SwR-STEAP1 CAR T cells: (a) and (c) The freshly transduced CAR T cells, and non-transduced (NT) T cells, were cultured for 21 days in the presence of 15 ng/ml recombinant human TGFβ (rhTGFβ). The absolute number of T cells was measured every 7 days (from day of transduction, day 0) by flow cytometry using counting beads. (b) and (d) The number of JK59 and JK69 CAR T cells measured every 7 days by flow cytometry using counting beads, after incubation with different concentrations of rhTGFβ, until day 21. Data represents the mean values #SEM of one healthy donor (in duplicates) from one representative experiment. The experiment was repeated two times. Data were analyzed by one-way ANOVA with Tukey's multiple comparisons test. *p<0.05; *p<0.01; *p<0.001; *p<0.0001; ns=not significant. (e) At the end of the proliferation assay (day 21), JK59 (top panel) and JK69 (bottom panel) CAR T cells cultured with 10 ng/ml and 15 ng/mL rhTGFβ, were co-cultured with 22Rv1, 22Rv1-KO, 22Rv1-TGFβ cell lines, and 22Rv1 cells cultured in the presence of 10 ng/mL rhTGFβ (22Rv1+rhTGFβ), at E:T ratio 1:1 for 48 hours. Lysis of target cells was measured by the intensity of Red-active-caspase3, using flow cytometry. The percentage of apoptotic target cells is represented in the graphs. Data represents the mean values±SEM of one healthy donor (in duplicates) from one experiment.

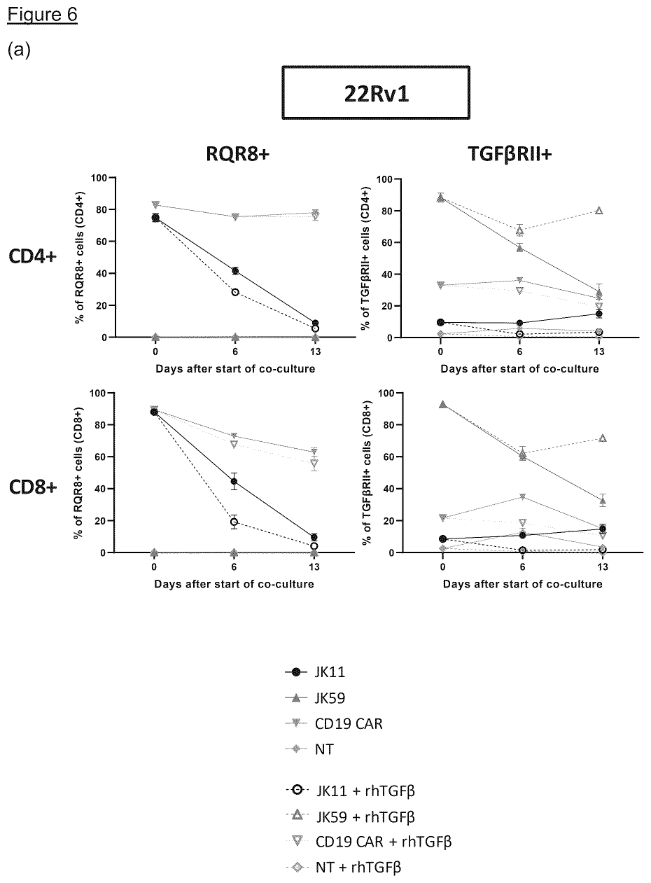

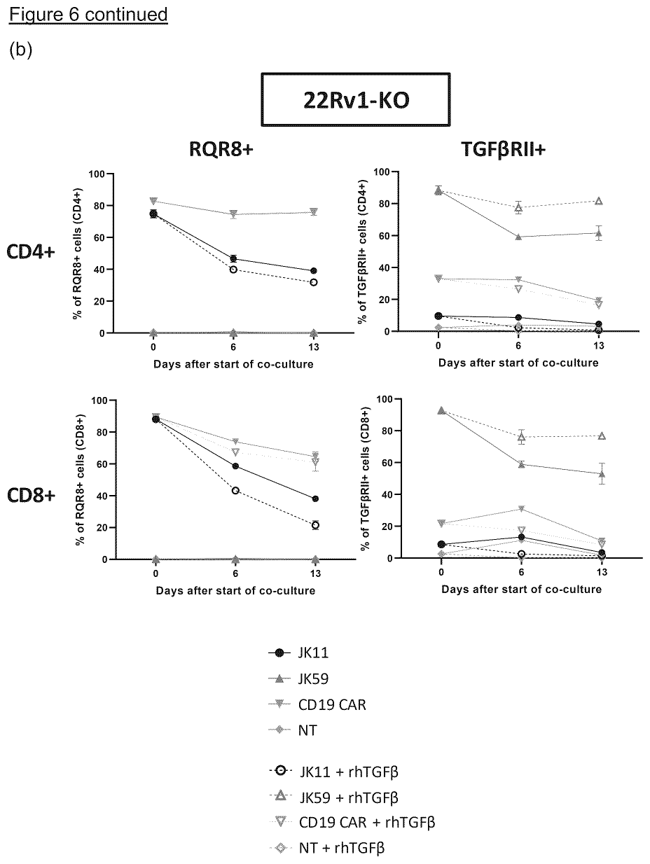

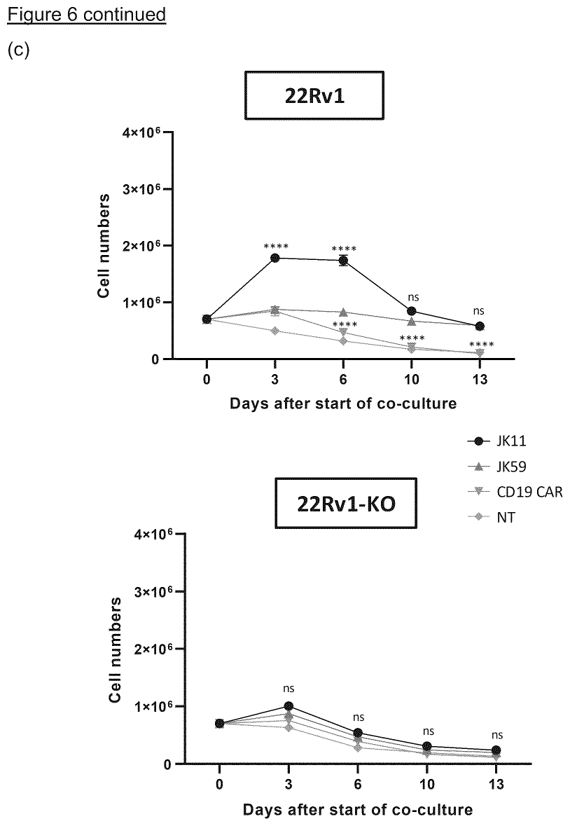

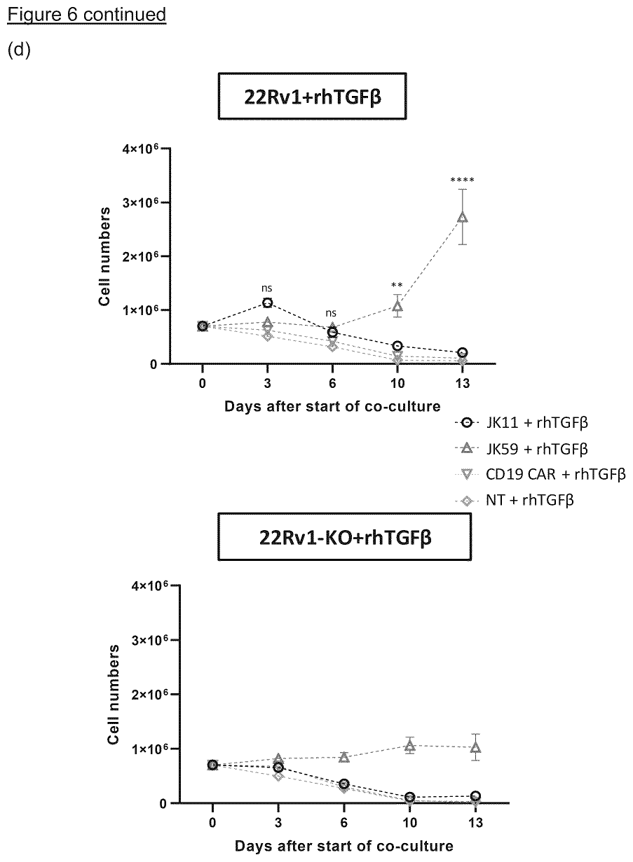

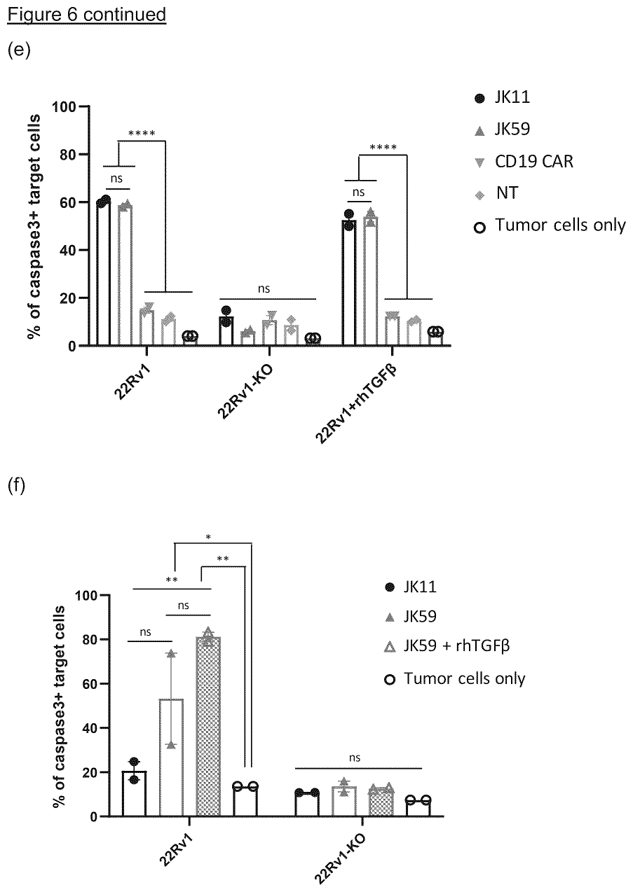

FIG. 6: TGFβ enhances the proliferation and functionality of SwR-STEAP1 CAR T cells after repeated in vitro stimulation with tumour cells: Freeze-thawed and activated JK11, JK59 and CD19 CAR T cells, and non-transduced (NT) control T cells, were co-cultured with irradiated (20 Gy) 22Rv1 and 22Rv1-KO target cells, in the absence or presence of 10 ng/ml rhTGFβ, at an E:T ratio of 1:1 for 3 to 4 days. Every 3 to 4 days, the T cell number was measured, and the same T cells were co-cultured again on freshly irradiated target cells for 3 to 4 more days until day 13. (a) and (b) Graphs representing the CAR expression of JK11, JK59 and CD19 CAR T cells, and non-transduced (NT) T cells following co-culture with 22Rv1 (a) and 22Rv1-KO (b) target cells at days 0, 6 and 13. The CAR expression of JK11 and CD19 CAR T cell was detected with the antibody recognizing the RQR8 sequence (left panels) and the CAR expression of JK59 CAR T cells was detected with the TGFβRII antibody (right panels); (c) and (d) Graphs representing the number of T cells following co-culture with 22Rv1 (c) or 22Rv1-KO (d) target cells at days 0, 3, 6, 10 and 13 in the absence (left panel) or presence (right panel) of 10 ng/ml rhTGFβ. Data in (c) and (d) represents the mean values±SEM of two healthy donors. Each co-culture with each donors' cells were performed in duplicate, and each duplicate pair was kept separate until the end of the experiment. Statistical significance was determined by two-way ANOVA with Tukey's multiple comparisons test. **p<0.01; ***p<0.001; ****p<0.0001; ns=not significant. (e) Before the start of the long-term co-culture (day 0), JK11, JK59 and CD19 CAR T cells, and NT T cells, were co-cultured with 22Rv1 and 22Rv1-KO target cells, and with 22Rv1 in presence of 10 ng/mL rhTGFβ, at an E:T ratio of 1:1 for 24 hours. (f) At the end of the long-term co-culture (day 13), JK11, JK59 CAR T cells, and JK59 CAR T cells cultured with 10 ng/mL rhTGFβ, each co-cultured with 22Rv1 target cells for 13 days, were co-cultured with 22Rv1 and 22Rv1-KO cells at E:T ratio 1:1 for 24 hours. In (e) and (f), lysis of target cells was measured by the intensity of Redactive-caspase3, using flow cytometry. The percentage of apoptotic target cells is represented in the graphs. Target cells cultured alone (tumour cells only) were included as controls to indicate the baseline of active caspase3. Data represents the mean values±SEM of two healthy donors. Each co-culture from each donor was done in duplicate and each duplicate pair was kept separate until the end of the experiment. In (e) and (f), statistical significances were determined by one-way ANOVA with Tukey's multiple comparisons test. *<p0.05; **p<0.01; ****p<0.0001; ns=not significant.

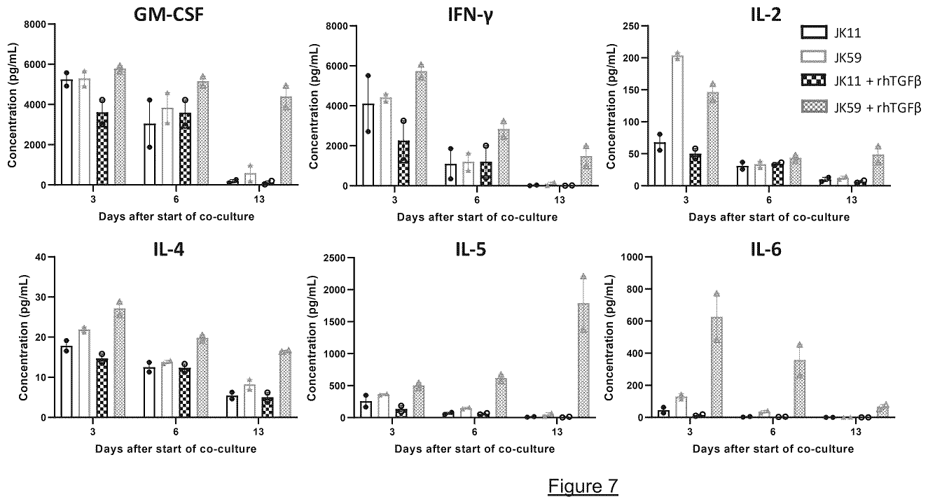

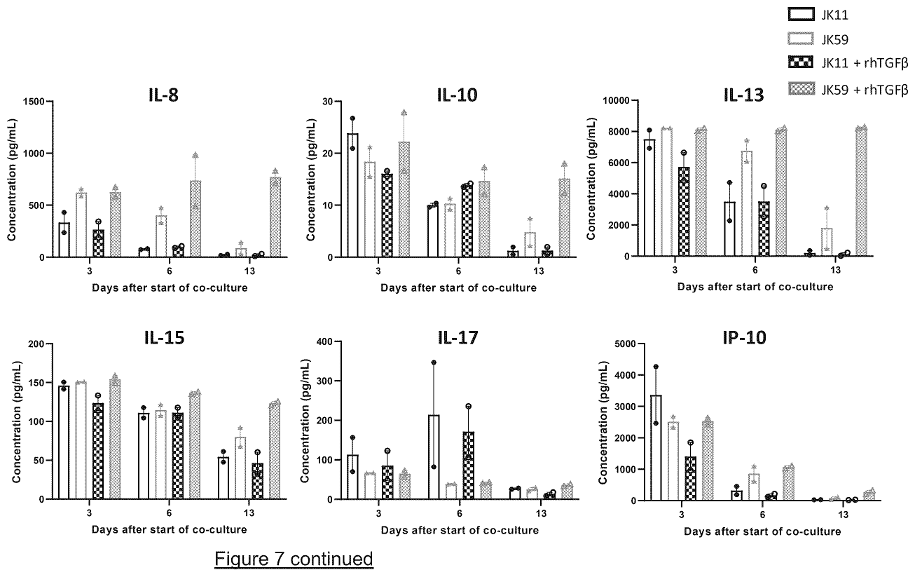

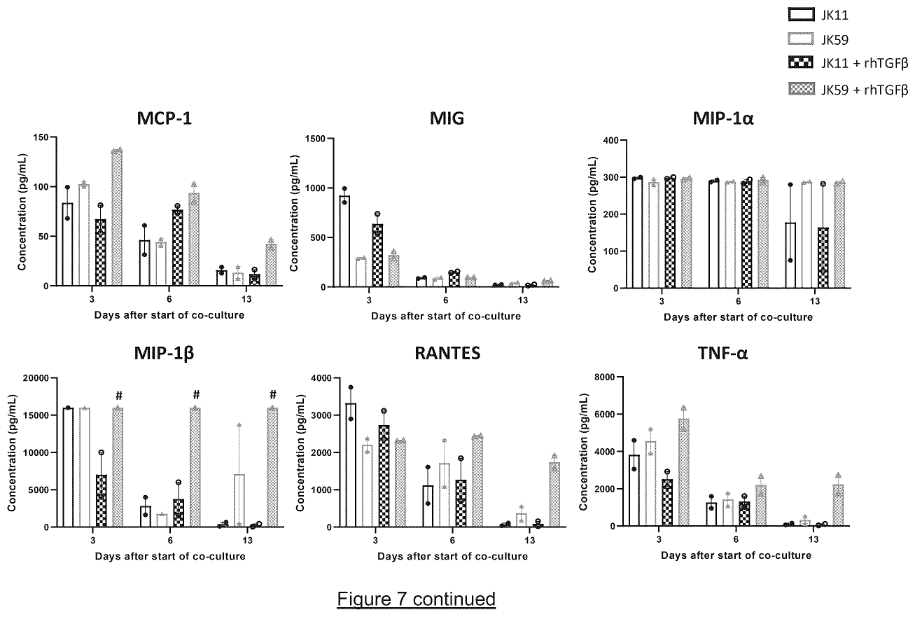

FIG. 7: TGFβ maintains a high cytokine profile in SwR-STEAP1 CAR T cells after repeated in vitro stimulation of STEAP1+ tumour cells: Luminex cytokine analyses were performed using T cells supernatants isolated at days 3, 6 and 13 from JK11 and JK59 CAR T cells, co-cultured with 22Rv1 target cells, in the absence or presence of 10 ng/ml rhTGFβ (as shown in FIG. 6). Data represents the mean values±SEM from two healthy donors. Each co-culture from each donor was done in duplicate, and each duplicate pair was kept separate throughout the experiment. The “#” symbol indicates that the values are higher than 16000 μg/mL.

FIG. 8: Analysis of STEAP1 and SwR-STEAP1 CAR T cell phenotyping after repeated in vitro stimulation of STEAP1+ tumour cells: Long-term co-culture was achieved by stimulating every 3 to 4 days the JK11, JK59 and CD19 CAR T cells, and NT T cells, with irradiated 22Rv1 and 22Rv1-KO target cells at an E:T ratio of 1:1 in the absence or presence of 10 ng/ml rhTGFβ. Before the start of the co-culture (day 0), at day 6, and day 13, CAR T cell phenotyping was assessed by flow cytometry. (a) and (b) The CD4+ and CD8+ T cell populations co-cultured with 22Rv1 target cells (a) or with 22Rv1-KO target cells (b) were assessed for surface expression of the activation marker CD25, and the checkpoint receptors PD1, LAG3, TIM3 and TIGIT. Background staining for each marker was identified using Fluorescence Minus One controls. Data represents the mean values±SEM from two healthy donors. Each co-culture was performed in duplicate, and these were kept separate throughout the assay.

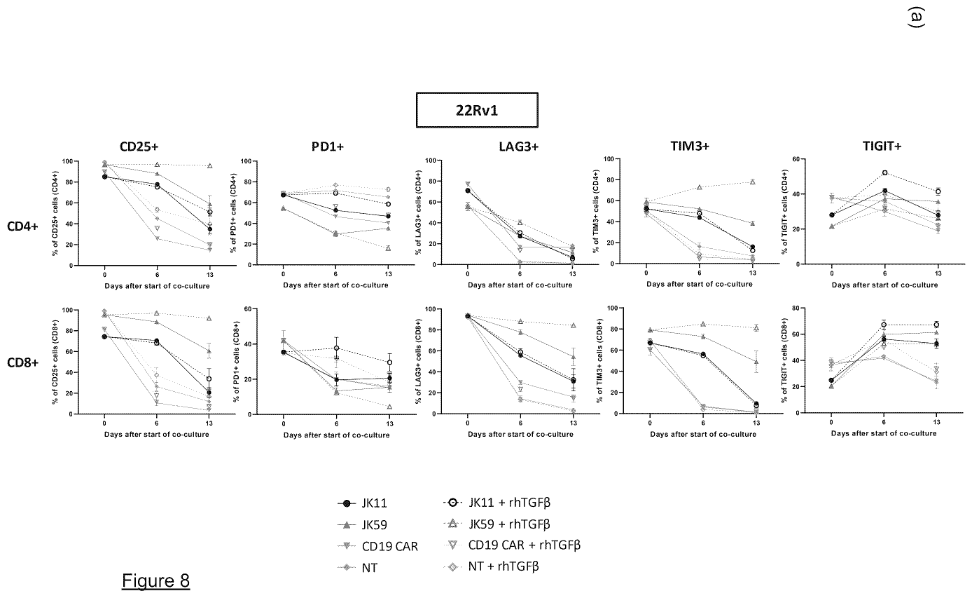

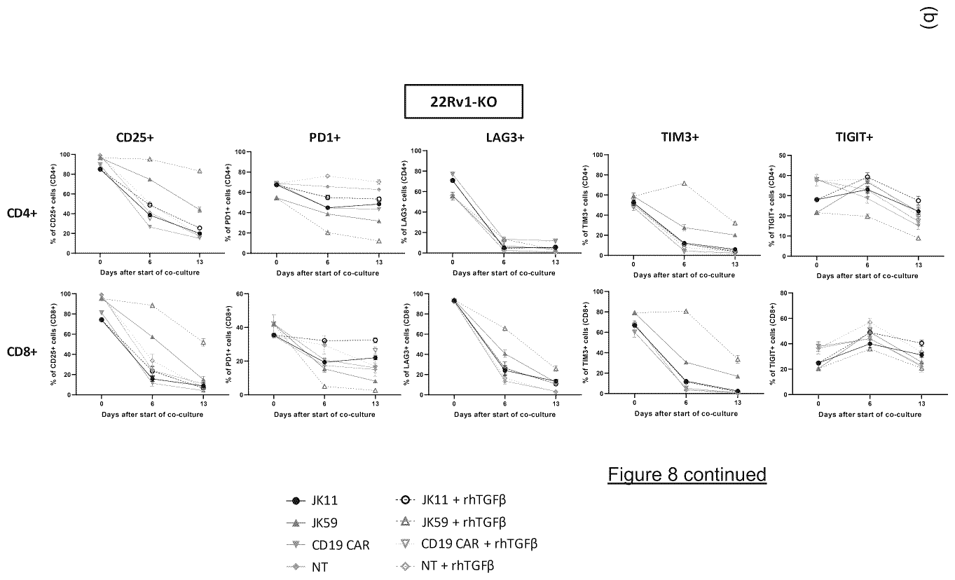

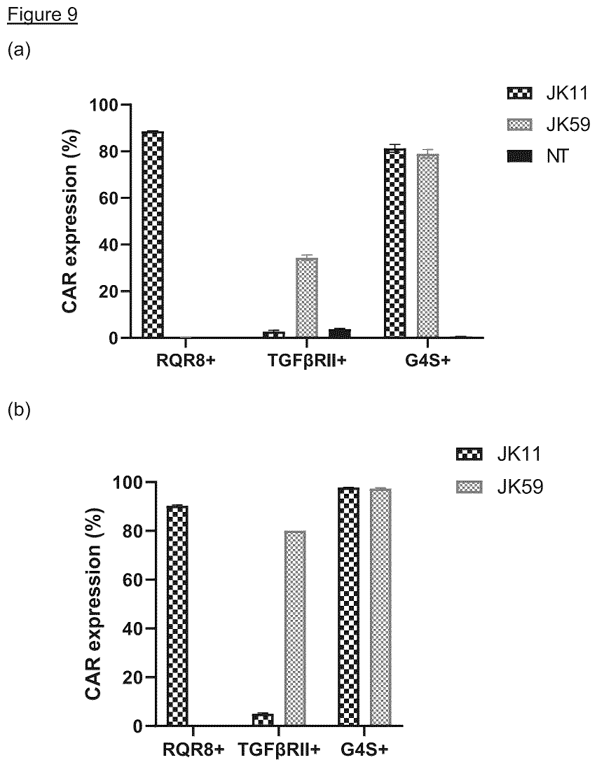

FIG. 9: STEAP1 CAR expression is comparable in STEAP1- and SwR-STEAP1-CAR T cells. Bar charts representing the CAR expression (CD3+ population) of freshly transduced (a) or thawed and activated for 3 days with anti-CD3/CD28 antibodies (b) JK11 and JK59 CAR T cells and non-transduced (NT) T cells. The CAR expression of JK11 and JK59 CAR T cells was detected with the antibody recognizing the G4S linker sequence, the switch receptor expression of JK59 CAR T cells was detected with the TGFβRII antibody, and the CAR expression of JK11 CAR T cells was detected with the antibody recognizing the RQR8 sequence. Data represent the mean values #SEM of one healthy donor, each in duplicates. The experiment was repeated 3 times with different healthy donors. Data were analyzed by one-way ANOVA with Tukey's multiple comparisons test.

FIG. 10: Analysis of IL-2/IL-15 intracellular signalling pathway upon rhTGFβ treatment in SwR-STEAP1 CAR T cells. Bar chart representing the phosphorylation level of STAT5 (pY694) in CD3+JK11 and JK59 CAR T cells, and in NT cells, treated with rhIL-2 or rhTGFβ for 15 min, or left untreated. Data represent the mean values of ±SEM of one healthy donor (in duplicate) from one representative experiment. The experiment was repeated two times.

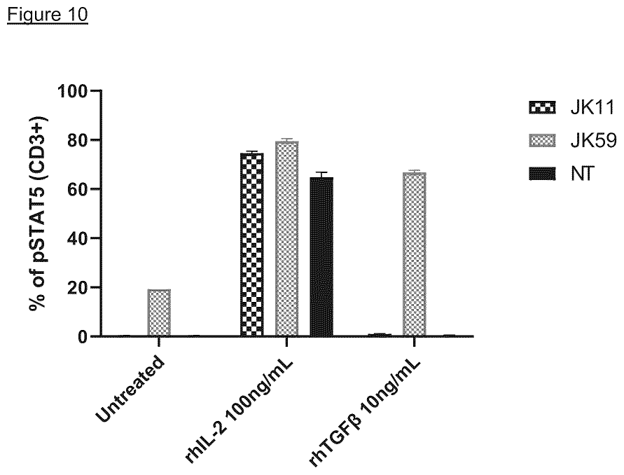

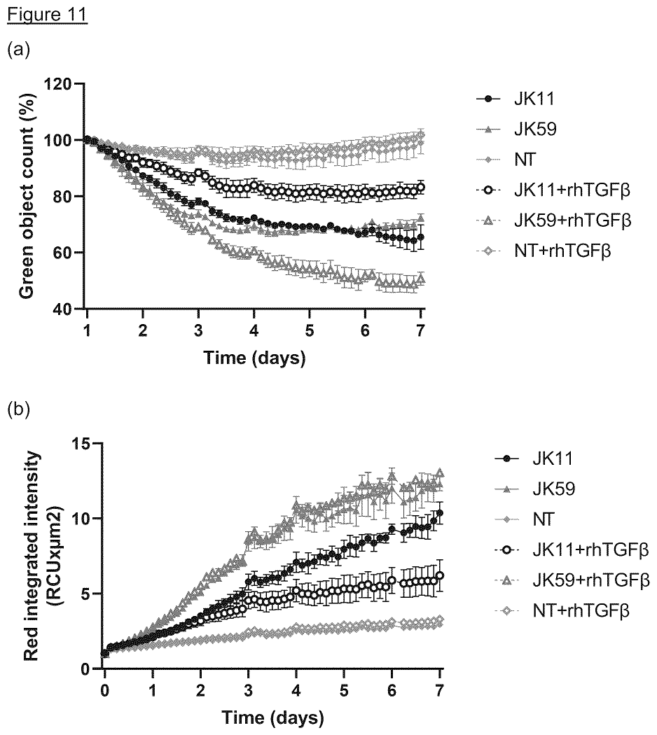

FIG. 11: Real-time monitoring of target killing and SwR-STEAP1 CAR T cells activation in TGFβ-rich environment. (a) Graph representing the killing of 22Rv1-GFP target cells by the CAR T cells, measured by the green object count (%) per image normalized to day 1 and to the level of GFP⁺ 22Rv1 target cells cultured alone for 7 days. (b) Graph representing the activation of T cells, measured by the red integrated intensity (RCUxμm2) per object average normalized to day 0. In (a) and (b), data represents the mean values±SEM of two different healthy donors, each in duplicate, from one representative experiment. The experiment was repeated three times, with four different healthy donors in total. Data were analyzed by two-way ANOVA with Tukey's multiple comparisons test.

FIG. 12: Enhanced tumor growth control in mice treated with SwR-STEAP1 CAR T cells in presence of rhTGFβ. Graphs representing the tumor growth, measured by bioluminescence imaging (a) or by calliper measurement (b) once a week. Data represent the mean values #SEM of each group of mice (JK11 n=4; JK59 n=5).

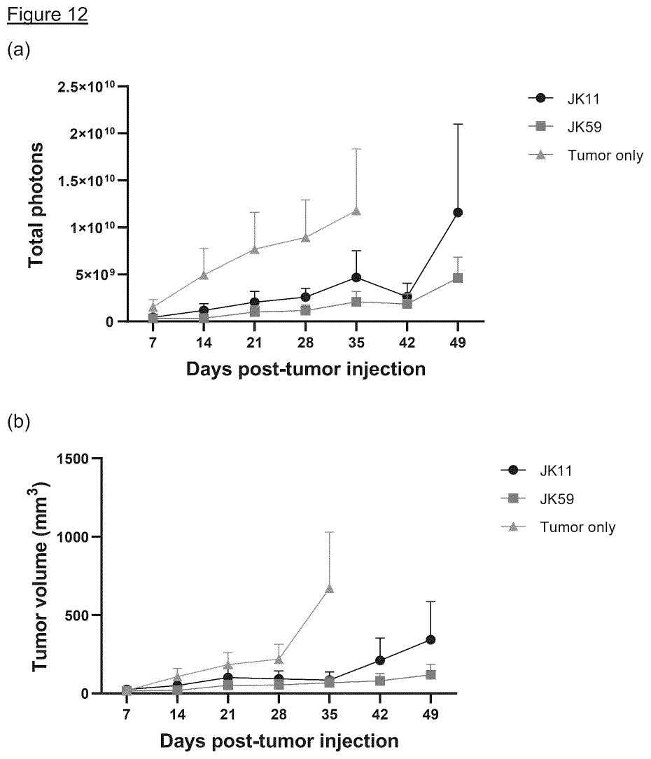

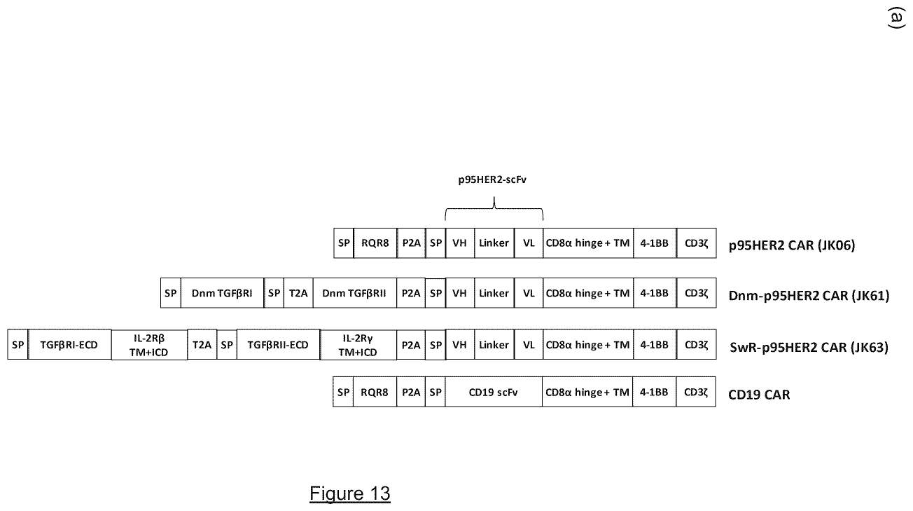

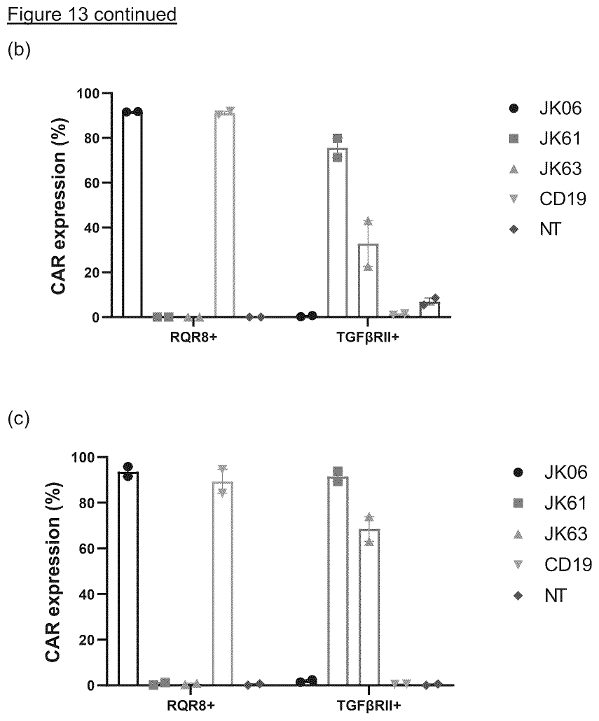

FIG. 13: SwR-p95HER2 CAR design and expression. (a) Schematic representations of the CAR constructs; parental p95HER2 CAR (JK06), control CD19 CAR, dominant negative mutant (Dnm)-p95HER2 CAR (JK61) with the TGFβ receptor (TGFβR) lacking the intracellular signalling domain, TGFβ-switch receptor (SwR)-p95HER2 CAR (JK63) with the extracellular domain (ECD) of the TGFβRI and TGFβRII subunits fused to the transmembrane domain (TM) and intracellular domain (ICD) of the IL-2/IL-15 receptor β and γ chains, respectively (IL-2Rβ and IL-2Rγ). (b) and (c) Bar charts representing the CAR expression of freshly transduced (b) or thawed and activated for 3 days with anti-CD3 and anti-CD28 antibodies (c) p95HER2 (JK06), Dnm-p95HER2 (JK61), SwR-p95HER2 (JK63), and CD19 CAR T cells and non-transduced (NT) T cells. The CAR expression of JK06 and CD19 CAR T cells was detected with the antibody recognizing the RQR8 sequence, and the CAR expression of JK61 and JK63 CAR T cells was detected with the TGFβRII antibody. Data represent the mean values #SEM of two healthy donor, each in duplicates. Data were analyzed by one-way ANOVA with Tukey's multiple comparisons test.

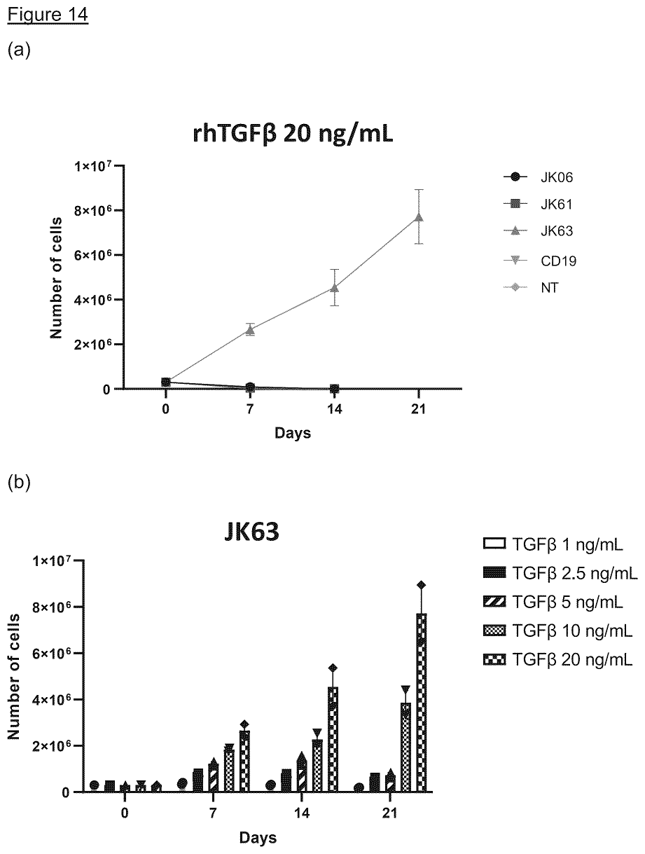

FIG. 14: TGFβ induces proliferation of the SwR-p95HER2 CAR T cells. (a) Graph representing the absolute cell number of freshly transduced CAR T cells, and non-transduced (NT) T cells cultured for 21 days in the presence of 20 ng/ml recombinant human TGFβ (rhTGFβ). JK61, CD19, and NT lines follow the JK06 line and are, therefore, not visible. (b) Bar chart representing the absolute number of JK63 CAR T cells cultured in presence of rhTGFβ at the different concentrations measured every 7 days. Data represent the mean values±SEM of two healthy donor (in duplicates) from one experiment. Data were analyzed by one-way ANOVA with Tukey's multiple comparisons test.

### EXAMPLES

The objective of the project was to develop a construct conferring tumour-targeting T cells with improved functionality in a TGFβ-rich environment. The results discussed below suggest that the described “switch receptor” (multichain chimeric receptor disclosed herein) fulfils this purpose at least for STEAP1 CAR T cells, both as measured by T cell proliferation, cytokine response, cytotoxicity and phenotypic development upon repeated/persisting antigen exposure.

Long-term assays, where the T cells were stimulated four times with target cells plus TGFβ, unexpectedly showed that the switch receptor: CAR T cells proliferated and evolved into effector memory cells, characterised by the expression of multiple activation markers, combined with low PD1 expression. Surprisingly, upon a fifth re-challenge with target cells, the switch receptor: CAR T cells showed retained and STEAP1-specific functionality.

### Example 1: Design of TGFβ-Switch Receptor and Co-Expression with STEAP1 CAR

The structure of the TGFβ receptor (TGFβR), a dimer, was compared to the intracellular domains (ICD, endodomains) of different stimulatory receptors. Based on this the ICD of the IL-2/IL-15 receptor β and γ chains were selected as a good candidate for a switch receptor (SwR) (i.e. a multichain chimeric receptor as defined herein) intended to be triggered by TGFβ, but confer a stimulatory signal to the T cells. A TGFβ-SwR was designed that encompasses the extracellular domain (ECD) of the TGFβRI and TGFβRII subunits fused to the transmembrane domain (TM) and ICD of the IL2/IL-15 receptor β and γ chains, respectively (FIG. 1a). The TGFβ-SwR was cloned into an anti-STEAP1 CAR construct JK11 (SEQ ID NO: 44), which incorporates the co-stimulatory 41BB domain. The resulting SwR-CAR construct was called JK59 (SEQ ID NOs: 47, 48 and 49, which refer to the amino acid sequence, nucleotide sequence and full vector sequence, respectively). For use as reference, a dominant negative mutant (Dnm) lacking the intracellular domain of the TGFβR was made and cloned this into the JK11 STEAP1 CAR. CAR constructs may confer a level of target-independent activity. As a control, a CAR comprising a CD19-specific scFv, cloned into the same backbone as the JK11 STEAP1 CAR, with identical spacer, transmembrane and intracellular domains (CD8 spacer/TM domain, 4-1BB, CD3ζ) was employed (FIG. 1a).

The expression of the JK11 STEAP1 CAR (RQR8-STEAP1_CD8_4-1BBQ) and CD19 CAR after PBMC transduction was measured by flow cytometry, using a mAb against the marker gene RQR8. The expression of JK59 (SwR-STEAP1_CD8_4-1BBC) and the Dnm-CAR (Dnm-STEAP1_CD8_4-1BBZ) was measured with a mAb against TGFβRII. Across four different donors, the mean JK59 expression was 28% in CD4⁺ T cells and 30% in CD8⁺ T cells. The Dnm-CAR was expressed in >80% of primary T cells, as was the JK11 and the CD19 CARs. T cells for clinical use need to be frozen, thawed and re-stimulated before infusion into patients. Interestingly, the expression of JK59 was markedly increased after freezing/thawing and two days re-stimulation, with a mean of 75% and 74% in CD4⁺ and CD8⁺ T cells, respectively (FIG. 1 b and c). The expression of JK11, the Dnm CAR and the CD19 CAR were largely unchanged, at 70-90%.

The expression level described above may not be directly comparable between the constructs, as the expression was detected with different mAbs, binding to different proteins (RQR8, TGFβRII). To obtain a comparable and direct CAR detection, and to assess if modifications to the spacer may affect the functionality, CAR constructs with an alternative spacer and TM domain derived from IgG1, but with mutations to avoid FcR binding we cloned (FIG. 2a). In these constructs, the expression of the CAR protein could be directly measured with a mAb against the IgG-spacer and compared between the constructs. The IgG-stainings demonstrated that the RQR8-STEAP1_IgG CAR construct (JK15, SEQ ID NO: 45) was expressed in 80% of transduced T cells, whereas the SwR-STEAP1_IgG CAR (JK69, SEQ ID NOs: 50, 51 and 52, which refer to the amino acid sequence, nucleotide sequence and full vector sequence, respectively) was expressed in 40% of T cells. The recorded percentages from IgG-stainings were in line with measurements based on mAbs against RQR8 and TGFβRII. Even for these constructs with IgG-spacers, the expression of the SwR-CAR (JK69) increased substantially after freezing/thawing, while the expression of the stand-alone CAR (JK15) was largely unchanged (FIGS. 2(b) and (c)).

### Example 2: STEAP1 Expression and TGFβ Secretion in Target Cell Lines

The functionality of the JK59 and Dnm-CAR constructs was investigated by using STEAP1^{+/-} prostate cancer cell lines as targets. As the expression levels may change during culture, new flow cytometry stainings of the cultured cells were performed. Cell lines 22Rv1 and LNCap were highly STEAP1⁺, whereas cell lines PC3 and DU145 were confirmed to be STEAP1 negative, as was a CRISPR/Cas STEAP1 knockout variant of 22Rv1 (22Rv1-KO). Further, 22Rv1 cells stably overexpressing TGFβ (22Rv1-TGFβ) were generated, which was co-expressed with EGFP. The TGFβ-EGFP construct was expressed in >98% of the 22Rv1TGFβ cells, and the STEAP1 expression was retained.

### Example 3: SwR STEAP1 CAR T Cells Exhibit Target Specific Cytokine Response

FIG. 3 shows a representative flow cytometry experiment assessing the TNFα and IFNγ response. Primary T cells were transduced with different CAR constructs (JK11, Dnm, JK59, CD19 CAR; all with CD8 spacer) or left non-transduced (NT), and then co-cultured over night with STEAP1 positive or negative target cells.

It was found that all three STEAP1 CAR T cell populations (JK59, Dnm, JK11) responded with production of TNFα and IFNγ, and that this response was dependent both on the expression of STEAP1 in target cells and on the STEAP1-specific scFv (compared to CD19-specific scFv). For all three STEAP1 CAR T variants, the percentage of TNFα⁺ T cells was higher for CD4⁺ (~50-70%) than CD8⁺ cells (~20-50%) (FIG. 3b), while the IFNγ⁺ percentage was higher for CD8⁺ cells (FIG. 3c). The functionality of the CD19 CAR control was confirmed, as the CD19 CAR T cells responded to CD19⁺ NALM-6 leukemia targets. Intriguingly, the percentage of TNFα⁺ and IFNγ⁺ T cells was similar for JK59 and the reference JK11 (FIG. 3b-c), even though the percentage of T cells expressing the SwR-CAR construct was only about 20% compared to the parental JK11 CAR, in these freshly transduced cells (FIG. 3a). This observation was made in repeated independent experiments with T cells from three donors. In most experiments, the percentage of TNFα⁺ T cells was highest for the Dnm-CAR, while the IFNγ response was similar for the JK59, Dnm and JK11 CAR.

### Example 4: SwR STEAP1 CAR T Cells Specifically Kill Target Cells

Next, the ability of the CAR T cells to kill tumour cells was investigated by assessing the induction of apoptosis in target cells, as measured by active caspase-3 (aCasp3). The different STEAP1 CAR T cells (JK59, Dnm, and JK11), as well as control T cells (CD19 CAR, NT) were co-cultured with STEAP1^{+/-} tumour cells at different effector-to-target (E:T) ratios.

We found that the induction of apoptosis was significantly increased in STEAP1⁺ targets co-cultured with the JK59 and Dnm CAR T cells, compared to the CD19 CAR and NT controls (FIG. 3d). The proportion of aCasp3⁺ targets was similar to the level obtained with JK11 STEAP1 CAR, indicating a fully retained killing capacity, even though the percentage of T cells expressing the CAR was lower (FIG. 3a). The killing increased, as expected, with increasing E: T ratios and reached 80% at an E:T ratio of 5:1 (FIG. 3d). The killing was STEAP1-specific, as no significant differences between the STEAP1 and CD19 CAR T cells were observed for 22Rv1-KO targets, and only CD19 CAR T cells killed NALM-6 target cells (FIG. 3d).

### Example 5: SwR Improves the Cytotoxic Capacity of STEAP1 CAR T Cells in a TGFβ-Rich Environment

It was hypothesized that the JK59 SwR-CAR T cells would show superior functionality in a TGFβ-rich environment. To investigate this hypothesis, different CAR T cell variants were co-cultured with STEAP1^{+/-} target cells, supplemented or not with rhTGFβ, and the induction of apoptosis was analysed by flow cytometry (FIG. 4a). The experiment demonstrated that all three STEAP1 CAR T cells killed target cells in a STEAP1-dependent manner, even in the presence of TGFβ. The JK59 CAR and Dnm-CAR gave significantly higher levels of killing compared to the parent JK11 CAR. This difference was more prominent for the 22Rv1 cell line overexpressing TGFβ (22Rv1-TGFβ), as compared to wild-type 22Rv1 cells, and for the cultures supplemented with rhTGFβ. For the JK11 CAR, the observed level of killing was lower in the cultures that contained TGFβ. The results were thus consistent with the hypothesis that the JK59 CAR T cells have superior functionality in a TGFβ-rich environment. A similar pattern was observed for the STEAP1 CAR with an IgG-based spacer. The JK69 CAR conferred T cells with a higher cytotoxic activity than the parent JK15 CAR, and the difference appeared most prominent for TGFβ-rich cultures (FIG. 4b).

### Example 6: SwR Gives Improved Expansion of STEAP1 CAR T Cells in a TGFβ-Rich Environment

It was investigated how the two SwR-CAR T cell variants (JK59 and JK69) responded to TGFβ, compared to the Dnm-CAR, the JK11 reference and the controls. To this aim, the proliferative capacity and survival of the T cells were monitored for 21 days, where the T cells were cultured with TGFβ, at concentrations ranging from 1.0-15 ng/ml. As shown in FIG. 5, the number of SwR-STEAP1 CAR T cells increased steadily over the entire observation period, resulting in a 15-fold expansion for JK59 and a 33-fold expansion for JK69, whereas none of the other CAR T cell groups showed any expansion at any time point (FIG. 5a, c). After 21 days, the SwR-STEAP1 CAR T cells still expanded in numbers, while no viable cells were detected for any of the other T cell populations. This applied to both SwR variants (JK59, FIG. 5a-b; JK69, FIG. 5c-d). The effect of TGFβ on the SwR CAR T cells was dose-dependent and increased gradually over the range of 1.0-15 ng/ml TGFβ (FIG. 5 b, d).

Next, we investigated whether the functionality of the SwR-STEAP1 CAR T cells was retained after long-term expansion in a TGFβ-rich environment, and if their functionality was impacted by the continued presence of TGFβ during encounter with target cells. The two variants SwR-STEAP1 CAR T cells were harvested after 21 days and co-cultured with STEAP1^{+/-} tumour cells (wild-type 22Rv1 and 22Rv1-KO), as well as with 22Rv1 cells overexpressing TGFβ (22Rv1-TGFβ) or supplemented with rhTGFβ (22Rv1⁺ rhTGFβ). As shown in FIG. 5e, the assay indicated that both JK59 and JK69 had retained and STEAP1-specific cytotoxic activity, giving induction of apoptosis in a high proportion (80%) of target cells. The continued presence of TGFβ did not affect the level of killing.

### Example 7: CAR T Cell Expansion and Functionality Upon Repeated Target Cell Exposure

It is known that continued antigen stimulation of CAR T cells may lead to T cell exhaustion or activation induced cell death (AICD). The SwR-ICD was derived from a domain shared by the receptors for IL2 and IL-15. While IL-15 is mainly considered to promote T cell survival, the continued stimulation of IL-2R may also give T cell exhaustion and AICD. It was investigated how the CAR T variants responded to continuous/repeated antigen exposure by subjecting the T cells to four serial stimulations over 2 weeks with irradiated 22Rv1 cells or 22Rv1-KO controls. The T cells were throughout the assay kept in culture with the irradiated feeder cells, which were replaced every 3-4 days. At “day 0”, the cells had been thawed and re-activated with anti-CD3/CD28 antibodies. The experiment was performed with two donors, each in duplicates, with/without supplemented rhTGFβ. At day 0, the expression levels of the CAR constructs with/without SwR were similar (~80-90%), as estimated by staining for RQR8 or TGFβRII (FIG. 6a-b). Cell counts from cultures without addition of rhTGFβ showed that the JK11 and JK59 CAR T cells survived until the end of the experiment, while the NT and CD19 CAR T controls did not (FIG. 6c). The number of JK11 cells increased during the first week, but then subsided, while the JK59 CAR T cells maintained stable numbers. The survival and expansion were STEAP1-dependent, as no T cells survived in the 22Rv1-KO cultures.

In cultures supplemented with rhTGFβ, the JK11 CAR T cells started decreasing in numbers after the second stimulation, and did not survive until day 13. By contrast, the JK59 CAR T cells increased substantially in numbers after the third and fourth stimulation (FIG. 6d). In the 22Rv1-KO cultures supplemented with rhTGFβ, the number of JK59 CAR T cells remained largely stable. The data thus indicated that the expansion of JK59 CAR T cells was dependent both on STEAP1 and TGFβ. The CAR expression decreased considerably for JK11 over the 14 days (30-40% at day 6, 5-10% at day 13), while it remained relatively high for JK59 (~60% at day 6, 70-80% at day 13; FIG. 6a-b)

The CAR T functionality was assessed by aCasp3 assays before start of co-culture (day 0) and after the four serial stimulations (day 13). At day 0, the JK11 and JK59 CAR T cells both showed potent STEAP1 specific cytotoxic activity, at similar levels (FIG. 6e). At day 13, JK11 T cells cultured with rhTGFβ had not survived, while JK11 T cells cultured without rhTGFβ showed modest cytotoxic activity (FIG. 6f). The SwR CAR T cells (JK59) showed a substantially higher cytotoxic activity, and those cultured with rhTGFβ killed the largest proportion of target cells (~80%; FIG. 6f).

The cytokine profile was characterised in multiplex assays (FIG. 7). The initial evaluation at day 3 showed that the JK11 CAR T cells secreted a wide range of cytokines upon 22Rv1 stimulation, and that this response was STEAP1-dependent. The cytokine response was Th1-weighted, with high levels of TNFα and IFNγ, and only marginal levels of IL-4 and IL-10. The CAR T cells also secreted GM-CSF, IL-15 and the chemokines IL-8, IP-10, MIG, MIP-1a, MIP-1B and RANTES, suggesting polyfunctionality and a potential to attract and support immune cells. Interestingly, the supplementation of TGFβ gave an attenuated cytokine response for JK11 CAR T cells, while the JK59 CAR T cells were resistant to this suppression.

After repeated stimulations with target cells, the cytokine response subsided in the JK11 CAR T cells, but persisted for the JK59 CAR T cells in the TGFβ-rich cultures (FIG. 7). At day 13, the measured levels for most cytokines were minimal in JK11 CAR T cultures, even after 22Rv1 stimulation without TGFβ, where the number of viable T cells were comparable to day 0 (FIG. 6c). In the JK59 CAR T cell cultures without supplemented TGFβ, most cytokines were still detected at day 13, whereas the levels were substantially higher in the TGFβ-rich cultures (FIG. 7).

Taken together, the results indicated that the effect of TGFβ was opposite on STEAP1 CAR T cells with/without expression of the SwR. The JK11 CAR T cells were suppressed by TGFβ, whereas the SwR CAR T cells (JK59) were stimulated, both regarding proliferation/survival, cytotoxicity and cytokine secretion. The data after 14 days (four stimulations) suggested that the functionality of the JK59 CAR T cells was intact, with no evidence of exhaustion or AICD.

### Example 8: CAR T Cell Phenotype Upon Repeated Target Cell Exposure

The evolution of the T cell phenotype in the cultures described above was investigated by stimulation four times with irradiated 22Rv1 or 22Rv1-KO cells. The flow cytometry analyses were performed for the same timepoints as the Luminex assays, i.e. at day 0, day 6 (after two simulations with 22Rv1/22Rv1-KO cells) and day 13 (3 days after the 4th stimulation).

The expression of checkpoint molecules/activation markers CD25, PD-1, LAG3, TIGIT and TIM3 were measured. At day 0, the T cells generally expressed high levels of these molecules, suggesting an activated T cell phenotype (FIG. 8a-b). After repeated stimulations, the JK11 CAR T cells expressed less CD25, LAG3, TIM3 and TIGIT, whereas their PD-1 level was relatively stable. By contrast, the JK59 CAR T cells supplemented with TGFβ gradually expressed less PD-1, but remained mostly CD25⁺, and also retained a considerably higher expression of LAG3 (in CD8⁺ T cells) and TIM3 (FIG. 8a). To a lesser extent, these differences also applied to JK59 CAR T cultures without TGFβ.

The proportion of single cells co-expressing the activation marker CD25 with checkpoint molecules PD-1, LAG3, TIGIT or TIM3 was determined. T cells expressing CD25, but not PD-1, may represent an activated, non-exhausted phenotype. The JK59 CAR T cells had a higher proportion of this phenotype, both for CD4⁺ and CD8⁺, compared to the JK11 CAR T cell population. The difference became larger during culture with TGFβ. At day 13, 82%/88% of CD4⁺/CD8⁺ JK59 CAR T cells cultured with 22Rv1-TGFβ were CD25⁺ PD-1, whereas only 25%/19% of the of CD4⁺/CD8⁺ JK11 CAR T cells had this phenotype. The proportions of single cells co-expressing CD25 with LAG3, TIGIT or TIM3 were also considerably higher in the JK59 CAR T cells. After 13 days culture with 22Rv1-TGFβ, 77%/82% of CD4⁺/CD8⁺ JK59 CAR T cells were CD25⁺ TIM3⁺, and 24%/48% of CD4⁺/CD8⁺ T cells were CD25⁺/TIGIT⁺. LAG3 was mainly expressed in CD8⁺ T cells, were 80% were CD25⁺ LAG3⁺. The expression of checkpoint molecules LAG3, TIGIT and TIM3 decreased over the 13 days in JK59 CAR T cells cultured with 22Rv1-KO plus TGFβ, and thus appeared STEAP1-dependent (FIG. 8a-b). The proportion of single cells co-expressing CD25 with LAG3, TIGIT or TIM3 was substantially lower at day 13 for JK59 CAR T cells cultured with 22Rv1-KO+rhTGFβ, compared to wildtype 22Rv1+rhTGFβ.

The T cells were, furthermore, classified into four differentiation subsets; naive, effector memory (EM), central memory (CM), or T effector memory re-expressing CD45RA (terminal differentiated; TEMRA). At day 0, ~80% of the JK11 and JK59 CAR T cells were CM cells. Upon stimulation with STEAP1⁺ 22Rv1 cells, the proportion of EM cells increased to ~80%, and the CM proportion decreased correspondingly. The CAR T differentiation from CM to EM was more prominent in CAR T cells cultured with STEAP1⁺ 22RV1 cells, compared to STEAP1 KO controls, and thus appeared to be partially STEAP1-dependent. The proportion of TEMRA was <2% and <13% in CD4⁺ and CD8⁺ T cells, respectively, throughout the 14 days for all CAR T variants co-cultured with STEAP1⁺ 22Rv1 cells. In JK59 CAR T cells cultured with 22Rv1-KO+rhTGFβ, the naive and TEMRA proportions increased over the 14 days.

The results indicated that most JK59 CAR T cells had an EM phenotype after four stimulations with 22Rv1+rhTGFβ and expressed multiple activation markers, but not PD-1. This observation suggested that the T cells were activated, but not exhausted, and was consistent with the data described above from functional T cells assays (Caspase3, flow cytometry cytokines, Luminex, cell counts).

### Example 9: Direct Detection of the STEAP1 CAR by Flow Cytometry with the Anti-Linker (G4S Repeat) Antibody

The CAR expression in JK11 or JK59 CAR T cells may not be comparable, as it was detected using two different antibodies, one against the marker RQR8 present in JK11, and one against the TGFβRII recognizing the switch receptor present in JK59. To compare the STEAP1 CAR expression in both JK11 and JK59 CAR T cells, and without modifying the spacer in the constructs, a commercially available antibody recognizing the linker sequence between the VH and VL sequences of the STEAP1 scFv was used. This antibody binds directly to the glycine-serine (G4S) repeat present in both JK11 and JK59 CAR constructs, which makes the CAR expression comparable.

T cells from healthy donors were activated for 2 days with anti-CD3/CD28 antibodies in presence of rhIL-2 at 100 IU/mL. T cells were transduced with retroviral supernatant, expanded and cryopreserved. The CAR expression was measured either 6 days post transduction or 3 days after thawing/activation by flow cytometry. The CAR expression was detected either with the antibody recognizing the RQR8 sequence on JK11 CAR T cells, or with the antibody recognizing the linker sequence (G4S repeat) between the VH and VL sequence on JK11 and JK59 CAR T cells. The SwR expression was detected with the TGFβRII antibody.

The staining using the G4S antibody revealed that the STEAP1 CAR expression was similar (~80% of total CD3+ cells) in both JK11 and JK59 freshly transduced T cells (FIG. 9a). The CAR expression was comparable in JK11 T cells using the antibody recognizing the RQR8 sequence, and the switch receptor was expressed on ~37% of the CD3+JK59 T cells, but absent in the JK11 T cells. The expression of the STEAP1 CAR detected with the G4S antibody was slightly better after freezing/thawing and activation of the CAR T cells (90% of CD3+ cells), while the expression of the switch receptor was significantly increased, as reported previously (FIG. 9b). This result demonstrated that the JK59 SwR-STEAP1 CAR construct retained the same CAR expression as its parental control JK11, despite the large size of the polycistronic construct.

### Example 10: IL-2/IL-15 Intracellular Signalling Pathway Activation Upon rhTGFβ Treatment in SwR-STEAP1 CAR T Cells

Signal transduction and activator of transcription 5 (STAT5) is a member of the Jak/STAT signal transduction pathway and is activated by different cytokines, including IL-2 and IL-15. Upon IL-2/IL-15 binding, STAT5 is phosphorylated, translocate into the nucleus and regulate the transcription of specific genes involved in many cellular processes, including the proliferation and differentiation of T cells. In the JK59 SwR-STEAP1 CAR construct, the intracellular domains of the IL-2/IL-15 receptor β and γ chains are fused to the extracellular domain of the TGFβ receptors I and II. This means that upon TGFβ treatment, TGFβ binds to its receptor and induces the IL-2/IL-15 signalling pathway through the phosphorylation of STAT5 at Y694. The activation of the IL-2/IL-15 signalling pathway upon TGFβ treatment was evaluated in the JK59 SwR-STEAP1 and JK11 STEAP1 CAR T cells by flow cytometry using a specific antibody recognizing pSTAT5 at Y694.

It was observed that in cells incubated with rhIL-2, the level of pSTAT5 increased significantly in both JK11 and JK59 CAR T cells as well as in non-transduced control cells (~70% of CD3⁺ cells), while no phosphorylation of STAT5 could be detected in untreated T cells. However, upon TGFβ treatment, only the JK59 CAR T cells showed a high level of pSTAT5 (~60%), compared to the JK11 CAR T cells or the NT T cells, where no pSTAT5 was detected (FIG. 10). This result confirmed the functionality of the switch receptor.

### Example 11: Real-Time Monitoring of Target Killing and SwR-STEAP1 CAR T Cells Activation in TGFβ-Rich Environment

The ability of STEAP1 (JK11) and SwR-STEAP1 (JK59) CAR T cells to kill target cells in presence or absence of rhTGFβ was investigated using the IncuCyte S3 real-time live cell analysis system. This system monitors cell death over time by the continuous imaging the GFP-labelled target cells, and T cells activation/proliferation by imaging of Red-labelled T cells.

FIG. 11 shows that STEAP1 and SwR-STEAP1 CAR T cells effectively eliminated the 22Rv1 cells at a similar level in absence of rhTGFβ compared to NT control cells who gave limited non-specific effects. However, in TGFβ-rich environment, the killing capacity of the JK11 STEAP1 CAR T cells was inhibited, while it was enhanced by the JK59 SwR-STEAP1 CAR T cells over the 7 days of analysis (FIG. 11a). The inhibition of the activity of the STEAP1 JK11 CAR T cells in presence of rhTGFβ was also demonstrated by the decrease of the Integrated intensity of the T cell clusters compared to the JK59 T cells, which was similar to the JK59 T cells cultured without rhTGFβ (FIG. 11b). The NT T cells did not show any activity against the target cells, as the integrated intensity remained very low at day 7 compared to day 0.

### Example 12: SwR-STEAP1 CAR T Cells Inhibit Tumor Growth In Vivo

It was observed in vitro that the JK59 SwR-STEAP1 CAR T cells killed the target cells with a higher efficiency in presence of TGFβ compared to the JK11 STEAP1 CAR T cells, and this difference was increased and could be visualized at a lower effector-to-target ratio. The efficacy of the JK59 SwR-STEAP1 CAR T cells was investigated in an in vivo model

In vivo, JK11 and JK59 CAR T cells both control the tumor growth at the conditions previously established for our CAR T studies. In the experiment shown in FIG. 12, a lower number of CAR T cells was injected, rhTGFβ was injected subcutaneously (s.c.) twice a week, in addition to rhIL-2. NXG mice were injected (s.c.) with 2×10⁶ of STEAP1*22Rv1-TGFβ-expressing cells, and treated with JK11 STEAP1 and JK59 SwR-STEAP1 CAR T cells 10 days and 17 days later, or left untreated (tumor only group). As observed previously, the tumor grew rapidly in the control mice, while the mice treated with JK11 or JK59 CAR T cells showed an inhibition of the tumor growth. However, a slight decrease of the tumor growth and tumor volume was observed in mice treated with the JK59 SwR STEAP1 CAR T cells compared to the mice treated with the JK11 CAR T cells from day 42 post-tumor injection (FIGS. 12a and b). The number of mice in each group was small (JK11 n=4 and JK59 n=5) and 1 mouse from JK11 group had to be euthanized at day 28, due to sickness (unrelated to CAR T cell side effects).

### Example 13: SwR-p95HER2 CAR Design and Expression

As for the TGFβ-SwR cloned into the STEAP1 CAR (JK11), a TGFβ-SwR was cloned into another CAR model, p95HER2 CAR (JK06). The TGFβ-SwR encompasses the extracellular domain (ECD) of the TGFβRI and TGFβRII subunits fused to the transmembrane domain (TM) and ICD of the IL2/IL-15 receptor β and γ chains, respectively (FIG. 13a). The TGFβ-SwR was cloned into an anti-p95HER2 CAR construct JK06, which incorporates the co-stimulatory 41BB domain. The resulting SwR-p95HER2 CAR construct was called JK63. For use as reference, a dominant negative mutant (Dnm) lacking the intracellular domain of the TGFβR was made and cloned this into the JK06 p95HER2 CAR, named JK61.

The expression of the JK06 p95HER2 CAR and CD19 CAR was measured by flow cytometry, using a mAb against the marker gene RQR8. FIG. 13b shows that JK06 and CD19 CAR T cells express around 90% of the CAR. The expression of the JK63 SwR-p95HER2 CAR and JK61 Dnm-p95HER2 CAR was measured with a mAb against TGFβRII. FIG. 13b shows that JK63 expression was 30% in CD3⁺ T cells, and that JK61 CAR was expressed in >70% of primary T cells. T cells for clinical use need to be frozen, thawed and re-stimulated before infusion into patients. As observed with the JK59 SwR-STEAP1 CAR, the expression of JK63 was markedly increased after freezing/thawing and two days re-stimulation, with a mean of 70% in CD3⁺ T cells (FIG. 13c). The expression of JK06 the CD19 CAR were largely unchanged (90%) while it slightly increased in JK61 T cells (90%).

### Example 14: SwR Improves the Expansion of p95HER2 CAR T Cells in a TGFβ-Rich Environment

As for the STEAP1 CAR T cells, it was investigated how the SwR-p95HER2 CAR T cells respond to TGFβ compared to the Dnm JK61 and the parental p95HER2 CAR T cells. For that, the proliferation capacity and the survival of the T cells was evaluated by culturing the cells for 21 days with rhTGFβ at different concentrations.

T cells from two healthy donors were activated and stimulated for 2 days with anti-CD3/CD28 antibodies and transduced with the JK06, JK61, JK63 or CD19 CAR constructs. On the day of the transduction (day 0), T cells were incubated with human recombinant TGFβ1 (rhTGFβ) at 1, 2.5, 5, 10, or 20 ng/ml. Media-containing rhTGFβ was refreshed twice a week until the end of the experiment. T cell proliferation was measured every 7 days until day 21 using 123 count eBeads™ and analyzed with flow cytometry.

FIG. 14a shows that the proliferation of JK63 SwR-p95HER2 CAR T cells increase over time in presence of rhTGFβ, compared to the JK06, JK61, CD19 or the NT T cells which did not expand at any time point. The JK63 SwR-p95HER2 T cell expansion is dose-dependent, as the cell number increased over the range of 1.0 to 20 ng/ml of rhTGFβ, as shown in FIG. 14b. This result is similar to what we observed with the JK59 SwR-STEAP1 CAR T cells.

### Methods

### Cell Lines, Primary T Cell Culture and Activation

The prostate cancer cell lines LNCaP, 22Rv1, PC3 and DU145, and the NALM-6 leukemia cell line were cultured as previously described (Jin, Y., et al., Development of STEAP1 targeting chimeric antigen receptor for adoptive cell therapy against cancer. Mol Ther Oncolytics, 2022. 26: p. 189-206). The Phoenix-AMPHO cell line was maintained in DMEM 4.5 g/L glucose, supplemented with 10% Hyclone FBS (Sigma Aldrich, Oslo, Norway) and 100 U/mL penicillin/streptomycin. Peripheral blood mononuclear cells (PBMCs) were isolated from healthy donor buffy coats using Lymphoprep (Axis-Shield, Oslo, Norway) and cultured in RPMI with 10% heat inactivated FBS and 100 U/mL penicillin/streptomycin. T cells from PBMCs were activated for 2 days on plates coated with 1 μg/mL anti-CD3 (clone OKT3, Biolegend, Oslo, Norway, 317347) and 1 μg/mL anti-CD28 (clone CD28.6, Thermo Fisher Scientific, Oslo, Norway, 16-0288-81), and cultured in the presence of 100 IU/mL rhIL-2 (R&D Systems, Abingdon, UK, 202-IL).

### Generation of 22Rv1-KO and 22Rv1-TGFβ Cell Lines

The STEAP1 gene was inactivated in the 22Rv1 cell line using a CRISPR/Cas9-mediated gene editing approach, as previously described (Jin et al., 2022, supra). A 22Rv1 cell line overexpressing TGFβ was generated using the pHAGE-TGFB1 (a gift from Gordon Mills & Kenneth Scott, Addgene, #116799) lentiviral vector. Lentiviral particles were produced as previously described (Jin et al., 2022, supra). 22Rv1 cells were transduced for 48 h in the presence of 4 μg/ml of polybrene, and expanded. TGFβ was co-expressed with the marker gene EGFP. Cells over-expressing TGFβ (GFP*) were sorted to 98% purity using a Sony SH800 cell sorter (Sony, Weybridge, UK).

### CAR Design

The JK11 STEAP1 CAR and the CD19 CAR were constructed as described previously (Jin et al., 2022, supra). The dominant-negative mutant TGFβ receptor-STEAP1 CAR (Dnm-CAR) was constructed by inserting the truncated human TGFβ receptor I (TGFβRI, UniProt P36897) and TGFβ receptor II (TGFβRII, UniProt W8DXL6) sequences, lacking the intracellular kinase domains and separated by a T2A self-cleaving peptide, into the JK11 CAR by InFusion cloning. The JK59 and JK69 switch receptor (SwR)-STEAP1 CARs were constructed by inserting the extracellular domain of TGFβRI linked in frame with the transmembrane domain (TM) and intracellular domain (ICD) of the β chain of IL-2/IL-15 receptor (IL-2Rβ), a T2A self-cleaving peptide, and the extracellular domain of the TGFβRII linked in frame with the TM and ICD of the common γ chain of IL-2 receptor (IL-2Rγ) upstream of the P2A sequence by InFusion cloning (FIG. 1a). For the JK15 STEAP1-IgG CAR and JK69 SwR-IgG-STEAP1 CAR, the STEAP1-scFv sequence is linked to a human IgG1 hinge-CH2CH3 and transmembrane domain. The TGFβ-SwR was also cloned into another CAR model, p95HER2 CAR (JK06). The resulting SwR-p95HER2 CAR construct was called JK63. For use as reference, a dominant negative mutant (Dnm) lacking the intracellular domain of the TGFβR was made and cloned this into the JK06 p95HER2 CAR, named JK61.

### Retroviral Vector Production

Retroviral vectors were prepared by transient transfection of Phoenix-AMPHO cells using XtremeGENE 9 DNA transfection reagent (Roche, Oslo Norway) according to the manufacturer's protocol. Briefly, 5.5*10⁶ phoenix-AMPHO cells were plated in 10 cm tissue-culture dishes. 24 h later, cells were co-transfected with 3.7 μg of the retroviral plasmids, together with the packaging plasmids and incubated at 37° C. overnight. Cells were then transferred to 32° C. and the viral supernatants were harvested at 48 h and 72 h after transfection, cleared by centrifugation, and frozen down at −80° C.

### CAR T Cell Production and Expansion

T cells were activated with anti-CD3/CD28 antibodies and cultured with 100 IU/mL rhIL-2 for 2 days. Activated T cells were transduced with CAR retroviral supernatants on non-treated 24-well plates precoated with Retronectin (Takara Bio, Göteborg, Sweden, T100B-TAK). For JK11, JK06, JK61, JK15, CD19 CAR and Dnm-CAR transductions, 0.3*10⁶ of activated T cells, resuspended in 1 mL of culture media supplemented with 200 IU/mL rhIL-2, were infected with 1 mL of retroviral supernatants, centrifuged at 900 g for 60 minutes at 32° C. and incubated at 37° C. Two days later, 1 ml of media was replaced with fresh media supplemented with 100 IU/mL rhIL-2 and transduced T cells were expanded for 4 more days. For JK59, JK69 and JK63 CAR transductions, 500 UL of retroviral supernatant was added on Retronectin-coated plates, centrifuged at 2000 g for 60 minutes at 32° C., removed and then 0.3*10⁶ of activated T cells were added on the plates and infected as described for the other CARs. 24 h later, media was removed completely, and the same transduction method was done 1 more time before media refreshment and expansion of CAR T cells. The expanded CAR T cells were frozen and stored in liquid N₂. Before the functional assays, T cells were thawed and re-activated for 2 days with anti-CD3 and anti-CD28 antibodies and with 100 IU/mL rhIL-2. Alternatively, where noted, freshly transduced T cells were used directly for experiments.

### TGFβ ELISA

Prostate cancer cell lines were cultured for 48 hours in 10% FBS culture media. To reduce the background of TGFβ present in FBS, the cells were serum starved in media containing 0.5% of FBS for 24 more hours. Supernatants were harvested, centrifuged to remove any debris and frozen down at 80° C. The Human TGF-β1 Quantikine ELISA kit (R&D Systems, Abingdon, UK, DB100C) was used to quantify the amount of active TGFβ present in the supernatants and secreted by the prostate cancer cell lines, according to the manufacturer's protocol.

### Flow Cytometry Instruments, Staining and Reagents

Flow cytometry analyses were performed on LSR II or Symphony instruments (BD Biosciences, Oslo, Norway), and data were analyzed using FlowJo™ Software (Tree Star Inc., Ashland, USA). For all surface staining, cells were stained with the appropriate surface antibodies, in presence of the Fixable Viability Dye eFluor™ 780 (Thermo Fisher Scientific, Oslo, Norway, 65-0865) to label the dead cells. JK11, JK06, JK15 or CD19 CAR expression was determined using RQR8-binding mAbs QBend10-AlexaFluor®488 (FAB7227G) or QBend10-PE (FAB7227P) (R&D Systems, Abingdon, UK). Dnm, JK59, JK69, JK61 and JK63 CAR expression was determined using a TGFβRII-APC REAfinity™ antibody (Miltenyi Biotec, Lund, Sweden, 130-115-025). The CAR expression of JK11 and JK59 was also detected using an antibody binding to the G4S linker sequence between the VH and VL sequences, rabbit mAb G4S linker (E702V)-Alexa Fluor®647 (Cell Signalling, 69782S). The CAR expression of the JK15 and JK69 CARs was determined using the R-Phycoerythin AffinityPure goat-anti-human IgG (H+L) secondary antibody (Jackson ImmunoResearch, Cambridge, UK, 109-116-088). For surface staining in the functionality assay, the following antibodies were used: CD3-Pacific Blue (317314), CD4-BV510 (741182), CD8-BV711 (301044) (all from Biolegend, Oslo, Norway). To detect the intracellular cytokines, T cells were fixed and permeabilized in CytoFix/Cytoperm solution (BD Biosciences, Oslo, Norway, 554714), according to the manufacturer's protocol, and stained with TNFα-PE (BD Biosciences, Oslo, Norway, 557068) and IFNγ-PECy7 (Thermo Fisher Scientific, Oslo, Norway, 25-73-19-82) antibodies. To detect the phosphorylation level of STAT5 (pY694), T cells were fixed and permeabilized according to the manufacturer's protocol, and stained with anti-STAT5 (pY694)-PE (BD Biosciences, 562077). For the phenotyping of CAR T cells the following antibodies were used: CD3-PerCPCy5.5 (317336), CD4-BV510 (741182), CD8-FITC (301050), CD25-BV605 (302632), LAG3-PECy7 (369310), TIM3-BV711 (345024), TIGIT-BV421 (372710), CCR7PEDazzle (353236) (all from Biolegend, Oslo, Norway), CD45RA-BUV496 (741182) and PD1-BV786 (563789) (both from BD Biosciences, Oslo, Norway). To assess the cell surface expression of STEAP1, prostate cancer cell lines were stained with the anti-STEAP1 mouse antibody (mAb) generated in our laboratory (Jin et al., 2022, supra), followed by the AlexaFluor® 647 AffinityPure goat-anti-mouse IgG (H+L) secondary antibody (Jackson ImmunoResearch, Cambridge, UK, 115-605-003).

### Functionality Assays (Killing and Cytokines Assay)

CAR T cells were, when specified, used freshly transduced, as described above, or thawed and reactivated with anti-CD3/CD28 antibodies for 2 days with 100 IU/mL rhIL-2. For the killing assays, CAR T cells pre-labelled with CellTrace™ Violet (Thermo Fisher Scientific, Oslo, Norway, C34557) were cocultured with target cells, at the specified Effector-to-Target (E:T) ratios, for 24 h or 48 h. The GaspGLOW™ Fluorescein active Caspase-3 (FITC-DEVD-FMK) (Thermo Fisher Scientific, Oslo, Norway, 88-7004-42) or the cleaved caspase-3 (Red-DEVD-FMK) (Abcam, Cambridge, UK, ab65617) was added into the co-culture to measure the target cell caspase 3 activation by flow cytometry. Cell death was detected using the Fixable Viability Dye eFluor™ 780. Where indicated, recombinant human TGFβ (rhTGFβ) was added to the co-culture.

To measure TNFα and IFNγ production, CAR T cells were co-cultured with target cells at an E:T ratio of 1:3 for 18 h in the presence of BD GolgiPlug™ (555029) and BD GolgiStop™ (554724) protein transport inhibitors (BD Biosciences, Oslo, Norway), according to the manufacturer's protocol. TNFα⁺ and IFNγ⁺ T cells were measured by flow cytometry using the specific antibodies.

### Proliferation Assay in Response to TGFβ

T cells were activated and stimulated for 2 days with anti-CD3/CD28 antibodies and transduced with the specific CAR constructs. On the day of the transduction (day 0). T cells were incubated with human recombinant TGFβ1 (rhTGFβ) (R&D System, Abingdon, UK, 7754-BH) at 1, 2.5, 5, 10, 15 or 20 ng/ml. Media-containing rhTGFβ was refreshed twice a week until the end of the experiment. T cell proliferation was measured every 7 days for 3 weeks using 123count eBeads™ (Thermo Fisher Scientific, Oslo, Norway, 01-1234-42) and analyzed by flow cytometry according to the manufacturer's instructions. At day 21, JK59 and JK69 CAR T cells were used in a 48 h killing assay at an E:T ratio of 1:1 with 22Rv1 target cells.

### Long-Term Co-Culture Assays

CAR T cells were thawed and re-activated with anti-CD3/CD28 antibodies for 2 days with 100 IU/mL rhIL2. 22Rv1 and 22Rv1-KO target cells were irradiated at 20 Gy and plated at 0.6*10⁶ cells/well in 12-well plates. 24 h later, 0.6*10⁶ CAR T cells (E:T ratio 1:1) were co-cultured with irradiated target cells, in the presence or absence of 10 ng/mL rhTGFβ, and without any additional rhIL-2 (day 0). 3 days later (day 3). T cells from these cultures were collected, labelled with trypan blue, and counted using a Countess™ Automated Cell Counter (Thermo Fisher Scientific, Oslo, Norway). They were then re-plated on freshly irradiated target cells at an E:T ratio of 1:1, in presence or absence of 10 ng/ml rhTGFβ. This assay was repeated every 3-4 days until day 13 (i.e. day 6, day 10, and day 13). At days 0, 6, and 13, expression of checkpoint markers on CAR T cells was measured by flow cytometry. At day 0 and day 13, CAR T cells were used in a 24 h killing assay at an E:T ratio of 1:1 with 22Rv1 target cells. Before each new coculture, supernatants were collected and stored at ~80° C. for multiplex cytokine analysis. Each coculture from each donor was done in duplicate, and each duplicate pair was kept separate until the end of the experiment.

### Multiplex Cytokine Analysis

Frozen supernatants from the long-term co-culture assay, collected at day 3, day 6 and day 13, were thawed and the secreted cytokines and chemokines were quantified using the Bio-Plex Pro Human Immunotherapy panel, 20-plex (Bio-Rad, Oslo, Norway, 12007975) according to the manufacturer's instructions. Samples were measured on a Luminex 200 instrument system (Bio-Rad, Oslo, Norway) and evaluated using the Bio-Plex Manager™ software, version 6.2. Supernatants from each donor were analyzed in duplicate. Each duplicate pair was kept separate from T cell stimulation until the end of the experiment.

### Phosphoflow Analysis

Cryopreserved JK11 and JK59 CAR T cells, and non-transduced T cells (NT) were thawed and activated for 3 days with anti-CD3 and anti-CD28 antibodies in presence of rhIL-2 at 100 IU/mL. Cells were rested for 24 hours in low serum-media (0.1% of FBS) and treated with either rhIL-2 at 100 ng/ml or rhTGFβ at 10 ng/ml for 15 minutes at 37° C. The samples were fixed by adding of Cytofix Buffer (BD) and incubated for 10 minutes at 37° C., then permeabilized in ice cold BD Phospho Perm Buffer III (BD) for 30 min at −20° C. and were then rehydrated in BD Stain Buffer (PBS-1% BSA). Cells were then stained with anti-CD3 and anti-STAT5 (pY694) and the proportion of positive cells was detected by flow cytometry.

### Real-Time Killing Assay

Cryopreserved JK11 and JK59 CAR T cells, and non-transduced T cells (NT) were thawed and activated for 3 days with anti-CD3 and anti-CD28 antibodies in presence of rhIL-2 at 100 IU/mL. The target cells 22Rv1-expressing nucleus-located GFP were irradiated at 20 Gy and seeded at a density of 1.2×10⁶ cells/mL in a 96-well plates. The following day, CAR T cells and NT T cells were labelled with the CellTracker Red CMTPX dye (ThermoFisher Scientific, C34552) according to the manufacturer's protocol, and added to the irradiated target cells at an effector-to-target (E:T) ratio of 1:3. When indicated, rhTGFβ at 10 ng/mL was added to the co-culture. Cells were real-time monitored every 3 hours for 7 days with IncuCyte® S3 live cell image system and images were analyzed using the IncuCyte® S3 built-in software.

### Subcutaneous In Vivo Prostate Cancer Xenograft Study

NXG (NOD-Prkdc^{scid}-IL2rg^{Tm1}/Rj) immunodeficient mice were bred in house, and 6- to 8-week-old mice were subcutaneously injected with 2×10⁶ 22Rv1-TGFβ-expressing cell line (firefly luciferase transduced) with 20% Matrigel on the hind leg. On day 10 and 17, mice were treated with 2×10⁶ JK11 (n=4) or JK59 (n=5) CAR T cells by i.v injection to the tail vein. 100 IU of rhIL-2 per gram body weight were injected twice a week (intraperitoneally (i.p.), and subcutaneously (s.c.) around the tumor), and 10 ng of rhTGFβ was injected twice a week (s.c.). Tumor growth was monitored once a week by bioluminescence IVIS imaging, in which anesthetized mice were injected i.p. with 150 mg/g body weight of D-luciferin (PerkinElmer Norway, Oslo, Norway) and imaged 12 min after luciferin injection. The tumors were also measured by calliper once per week.

### STATISTICAL ANALYSIS

All statistical analysis was performed using GraphPad Prism v9 (GraphPad Software Inc.). The differences among groups for all experiments were determined with a one- or two-way ANOVA followed by Tukey's multiple comparisons test. p-values<0.05 were considered as significant.

1. A polycistronic nucleic acid encoding: (i) a multichain chimeric receptor and; (ii) a chimeric antigen receptor (CAR) or a T-cell receptor (TCR) comprising an alpha chain and/or beta chain, wherein:
(A) the multichain chimeric receptor comprises:
(a) a first polypeptide comprising: (i) an extracellular domain comprising the extracellular domain of TGFβRI or a functional variant thereof; (ii) a transmembrane domain; and (iii) an endodomain comprising an intracellular signalling domain of IL-2Rβ or a functional variant thereof; and
(b) a second polypeptide comprising: (i) an extracellular domain comprising the extracellular domain of TGFβRII or a functional variant thereof; (ii) a transmembrane domain; and (iii) an endodomain comprising an intracellular signalling domain of IL-2Rγ or a functional variant thereof; or
(B) the multichain chimeric receptor comprises:
(a) a first polypeptide comprising: (i) an extracellular domain comprising the extracellular domain of TGFβRII or a functional variant thereof; (ii) a transmembrane domain; and (iii) an endodomain comprising an intracellular signalling domain of IL-2Rβ or a functional variant thereof; and
(b) a second polypeptide comprising: (i) an extracellular domain comprising the extracellular domain of TGFβRI or a functional variant thereof; (ii) a transmembrane domain; and (iii) an endodomain comprising an intracellular signalling domain of IL-2Rγ or a functional variant thereof.

2. The polycistronic nucleic acid of claim 1, wherein the transmembrane domain of the first polypeptide comprises or consists of the transmembrane domain of IL-2Rβ or a functional variant thereof.

3. The polycistronic nucleic acid of claim 1 or 2, wherein the transmembrane domain of the second polypeptide comprises or consists of the transmembrane domain of IL-2Rγ or a functional variant thereof.

4. The polycistronic nucleic acid of any one of claims 1 to 3, wherein the CAR comprises: (i) an extracellular domain comprising an antigen binding domain; (ii) a transmembrane domain; and (iii) an endodomain comprising intracellular signalling domain.

5. The polycistronic nucleic acid of any one of claims 1 to 4, wherein the multichain chimeric receptor and CAR or TCR are encoded in a single open reading frame, wherein adjacent encoding sequences are separated by a sequence encoding a self-cleaving peptide.

6. The polycistronic nucleic acid of any one of claims 1 to 5, wherein the nucleic acid encodes in order from 5′ to 3′:
(a) the first polypeptide of the multichain chimeric receptor;
(b) a first self-cleaving peptide (e.g. T2A or a functional variant thereof);
(c) the second polypeptide of the multichain chimeric receptor;
(d) a second self-cleaving peptide (e.g. P2A or a functional variant thereof); and
(e) the CAR or the TCR alpha chain or beta chain; and
optionally when the nucleic acid encodes a TCR alpha chain and beta chain
(f) a third self-cleaving peptide; and
(g) the TCR chain not encoded in (e).

7. The polycistronic nucleic acid of claim 5 or 6, wherein each self-cleaving peptide is independently selected from the group consisting of T2A, P2A, E2A and F2A or a functional variant thereof, preferably the group consisting of T2A and P2A or a functional variant thereof.

8. The polycistronic nucleic acid of any one of claims 5 to 7, wherein the nucleotide sequence encoding each self-cleaving peptide is codon-optimised for expression in human cells.

9. The polycistronic nucleic acid of any one of claims 5 to 8, wherein the nucleotide sequence encoding each self-cleaving peptide is different.

10. The polycistronic nucleic acid of any one of claims 5 to 9, wherein the amino acid sequence of each self-cleaving peptide is different.

11. The polycistronic nucleic acid of any one of claims 1 to 10, wherein the polycistronic nucleic acid is tricistronic.

12. The polycistronic nucleic acid of any one of claims 1 to 11, wherein the nucleic acid encodes:
(A) a multichain chimeric receptor comprising:
(a) a first polypeptide comprising: (i) an extracellular domain comprising the extracellular domain of TGFβRI or a functional variant thereof; (ii) a transmembrane domain; and (iii) an endodomain comprising an intracellular signalling domain of IL-2Rβ or a functional variant thereof; and
(b) a second polypeptide comprising: (i) an extracellular domain comprising the extracellular domain of TGFβRII or a functional variant thereof; (ii) a transmembrane domain; and (iii) an endodomain comprising an intracellular signalling domain of IL-2Rγ or a functional variant thereof; and
(B) a CAR comprising: (i) an extracellular domain comprising an antigen binding domain; (ii) a transmembrane domain; and (ii) an endodomain comprising intracellular signalling domain.

13. The polycistronic nucleic acid of any one of claims 1 to 12, wherein the first polypeptide of the multichain chimeric receptor comprises an amino acid sequence as set forth in SEQ ID NO: 14 or functional variant thereof comprising an amino acid sequence with at least 80% sequence identity to an amino acid sequence as set forth in SEQ ID NO: 14.

14. The polycistronic nucleic acid of any one of claims 1 to 13, wherein the second polypeptide of the multichain chimeric receptor comprises an amino acid sequence as set forth in SEQ ID NO: 15 or functional variant thereof comprising an amino acid sequence with at least 80% sequence identity to an amino acid sequence as set forth in SEQ ID NO: 15.

15. The polycistronic nucleic acid of any one of claims 1 to 14, wherein the antigen binding domain of the CAR or the TOR binds to a tumour associated antigen.

16. The polycistronic nucleic acid of claim 15, wherein the tumour associated antigen is up-regulated in any one or more of prostate cancer, Ewing sarcoma, lung cancer, bladder cancer, breast cancer, pancreatic cancer, glioblastoma, ovarian cancer, leukaemia, lymphoma and head and neck cancer.

17. The polycistronic nucleic acid of claim 15 or 16, wherein the tumour associated antigen is STEAP-1.

18. The polycistronic nucleic acid of any one of claims 1 to 17, wherein the CAR is an anti-STEAP-1 CAR.

19. The polycistronic nucleic acid of claim 18, wherein the anti-STEAP-1 CAR comprises an antigen-binding domain comprising a V_{H} region and a V_{L} region, each comprising three CDR sequences, wherein:
a) CDRs 1, 2 and 3 of the V_{L} region have the amino acid sequences of SEQ ID NOs: 18, 19 and 20, respectively; and
b) CDRs 1, 2 and 3 of the V_{H} region have the amino acid sequences of SEQ ID NOs: 21, 22 and 23, respectively; and
wherein one or more of said CDR sequences may optionally be modified by substitution, addition and/or deletion of 1 to 3 amino acids.

20. The polycistronic nucleic acid of claim 18 or 19, wherein the anti-STEAP-1 CAR comprises an amino acid sequence as set forth in any one of SEQ ID NOs: 39-46 (preferably SEQ ID NO: 44 or 45) or a functional variant thereof comprising an amino acid sequence with at least 95% sequence identity to an amino acid sequence as set forth in SEQ ID NOs: 39-46 (preferably SEQ ID NO: 44 or 45).

21. The polycistronic nucleic acid of any one of claims 1 to 20, wherein the nucleic acid encodes an amino acid sequence as set forth in SEQ ID NO: 47 or 50 or a functional variant thereof comprising an amino acid sequence with at least 95% sequence identity to an amino acid sequence as set forth in SEQ ID NO: 47 or 50.

22. The polycistronic nucleic acid of claim 21, wherein the nucleic acid comprises a nucleotide sequence as set forth in in SEQ ID NO: 48 or 51 or a nucleotide sequence with at least 95% sequence identity to a nucleotide sequence as set forth in SEQ ID NO: 48 or 51.

23. A vector, preferably a viral vector (e.g. a retroviral vector), comprising the polycistronic nucleic acid of any one of claims 1 to 22, optionally wherein the vector comprises a nucleotide sequence as set forth in SEQ ID NO: 49 or 52 or a nucleotide sequence with at least 95% sequence identity to a nucleotide sequence as set forth in SEQ ID NO: 49 or 52.

24. An immune effector cell:
(A) comprising the polycistronic nucleic acid of any one of claims 1 to 22 or the vector of claim 23, wherein the immune effector cell expresses: (i) a multichain chimeric receptor as defined in any one of claims 1 to 3 or 12 to 14; and (ii) a CAR as defined in any one of claim 4 or 15 to 20 or a TOR as defined in any one of claim 1, 15 or 16 at its surface; or
(B) that expresses: (i) a multichain chimeric receptor as defined in any one of claims 1 to 3 or 12 to 14; and (ii) an anti-STEAP-1 CAR, optionally as defined in claim 19 or 20.

25. The immune effector cell of claim 24, wherein the immune effector cell is a human T cell or a human NK cell.

26. A cell population comprising the immune effector cell of claim 24 or 25, optionally wherein the cell population is frozen or wherein the cell population is an activated and optionally expanded cell population obtained from immune effector cells that have been frozen and thawed.

27. A pharmaceutical composition comprising the immune effector cell of claim 24 or 25 or the cell population of claim 26 and a pharmaceutically acceptable carrier or excipient.

28. The immune effector cell of claim 24 or 25, the cell population of claim 26 or the pharmaceutical composition of claim 27, for use in therapy.

29. The immune effector cell of claim 24 or 25, the cell population of claim 26 or the pharmaceutical composition of claim 27, for use in the treatment of cancer, preferably STEAP1-positive prostate cancer.

30. The immune effector cell, cell population or pharmaceutical composition for use according to claim 29, wherein said prostate cancer is metastatic prostate cancer or said prostate cancer is castration-resistant.

31. A method for increasing the expression of a multichain chimeric receptor and/or a CAR at the surface of immune effector cells in a cell population, wherein the multichain receptor is as defined in any one of claims 1 to 3 or 12 to 14 and the CAR is as defined in any one of claim 4 or 15 to 20, the method comprising:
(a) providing a cell population comprising the immune effector cell of claim 24 or 25, wherein the immune effector cell comprises the polycistronic nucleic acid of any one of claims 1 to 22 or the vector of claim 23;
(b) freezing the cell population of (a);
(c) thawing the frozen cell population of (b); and
(d) activating and optionally expanding the thawed cell population of (c).

32. A tumour-infiltrating lymphocyte (TIL) or allogeneic natural killer (NK) cell expressing a multichain chimeric receptor as defined in any one of claims 1 to 3 or 12 to 14.

33. The TIL or allogeneic NK cell of claim 32 for use in therapy.

34. The TIL or allogeneic NK cell of claim 32 for use in treating cancer, optionally wherein the cancer is selected from melanoma, non-small cell lung cancer, ovarian cancer, head and neck cancer, colorectal cancer, liver cancer, breast cancer, soft tissue sarcoma, pancreatic cancer, cervical cancer, renal cancer, gastric cancer, osteosarcoma, oesophageal cancer, glioblastoma, thyroid cancer, neuroendocrine cancer, mesothelioma, bladder cancer, prostate cancer, multiple myeloma, small cell lung cancer and endometrial cancer.


SEQUENCE LISTING (59 sequences)

SEQ ID NO: 1 - protein, 8 aa, synthetic construct
  source 1..8: note = X is any amino acid; note = 2A consensus motif
DXEXNPGP

SEQ ID NO: 2 - protein, 21 aa, synthetic construct
  source 1..21: note = T2A
GSGEGRGSLLTCGDVEENPGP

SEQ ID NO: 3 - protein, 22 aa, synthetic construct
  source 1..22: note = P2A
GSGATNFSLLKQAGDVEENPGP

SEQ ID NO: 4 - protein, 23 aa, synthetic construct
  source 1..23: note = E2A
GSGQCTNYALLKLAGDVESNPGP

SEQ ID NO: 5 - protein, 25 aa, synthetic construct
  source 1..25: note = F2A
GSGVKQTLNFDLLKLAGDVESNPGP

SEQ ID NO: 6 - DNA, 63 bp, synthetic construct
  source 1..63: mol_type = other DNA; note = Codon optimised T2A
ggctcaggagaaggcaggggctccctgctcacatgcggggatgtggaagagaacccaggacca

SEQ ID NO: 7 - DNA, 66 bp, synthetic construct
  source 1..66: mol_type = other DNA; note = Codon optimised P2A
ggaagtggcgccaccaatttctcattgcttaagcaggccggggacgtggaggaaaacccagggccc

SEQ ID NO: 8 - protein, 126 aa, Homo sapiens
  source 1..126: note = TGFbetaRI exodomain
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 9 - protein, 166 aa, Homo sapiens
  source 1..166: note = TGFbetaRII exodomain
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 10 - protein, 286 aa, Homo sapiens
  source 1..286: note = IL-2Rbeta ICD
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 11 - protein, 86 aa, Homo sapiens
  source 1..86: note = IL-2Rgamma ICD
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 12 - protein, 25 aa, Homo sapiens
  source 1..25: note = IL-2Rbeta TMD
IPWLGHLLVGLSGAFGFIILVYLLI

SEQ ID NO: 13 - protein, 21 aa, Homo sapiens
  source 1..21: note = IL-2Rgamma TMD
VVISVGSMGLIISLLCVYFWL

SEQ ID NO: 14 - protein, 437 aa, synthetic construct
  source 1..437: note = TGFbetaRI:IL-2Rbeta TM+ ICD
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 15 - protein, 273 aa, synthetic construct
  source 1..273: note = TGFbetaRII:IL-2Rgamma TM+ ICD
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 16 - protein, 5 aa, synthetic construct
  source 1..5: note = linker
GGGGS

SEQ ID NO: 17 - protein, 20 aa, synthetic construct
  source 1..20: note = linker
GGGGSGGGGSGGGGSGGGGS

SEQ ID NO: 18 - protein, 17 aa, synthetic construct
  source 1..17: note = VLCDR1
KSSQSLLYRSNQKNYLA

SEQ ID NO: 19 - protein, 7 aa, synthetic construct
  source 1..7: note = VLCDR2
WASTRES

SEQ ID NO: 20 - protein, 9 aa, synthetic construct
  source 1..9: note = VLCDR3
QQYYNYPRT

SEQ ID NO: 21 - protein, 11 aa, synthetic construct
  source 1..11: note = VHCDR1
GYSITSDYAWN

SEQ ID NO: 22 - protein, 18 aa, synthetic construct
  source 1..18: note = VHCDR2
GYISNSGSTSYNPSLKSR

SEQ ID NO: 23 - protein, 15 aa, synthetic construct
  source 1..15: note = VHCDR3
ERNYDYDDYYYAMDY

SEQ ID NO: 24 - protein, 15 aa, synthetic construct
  source 1..15: note = VHCDR3 v1
ERNYDYEDYYYAMDY

SEQ ID NO: 25 - protein, 15 aa, synthetic construct
  source 1..15: note = VHCDR3 v2
ERNYDYDEYYYAMDY

SEQ ID NO: 26 - protein, 15 aa, synthetic construct
  source 1..15: note = VHCDR3 v3
ERNYDYEEYYYAMDY

SEQ ID NO: 27 - protein, 124 aa, synthetic construct
  source 1..124: note = VH
DVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSS

SEQ ID NO: 28 - protein, 113 aa, synthetic construct
  source 1..113: note = VL
DIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIK

SEQ ID NO: 29 - protein, 257 aa, synthetic construct
  source 1..257: note = scFv
DVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIK

SEQ ID NO: 30 - protein, 45 aa, Homo sapiens
  source 1..45: note = CD8alpha hinge
SDPTTTPAPRPPTPAPTIASQPLSLRPEACRPAAGGAVHTRGLDF

SEQ ID NO: 31 - protein, 240 aa, Homo sapiens
  source 1..240: note = native IgG hinge
SDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 32 - protein, 239 aa, synthetic construct
  source 1..239: note = IgGdelta hinge
SDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 33 - protein, 27 aa, Homo sapiens
  source 1..27: note = CD28 TMD
FWVLVVVGGVLACYSLLVTVAFIIFWV

SEQ ID NO: 34 - protein, 27 aa, Homo sapiens
  source 1..27: note = CD8alpha TMD
ACDIYIWAPLAGTCGVLLLSLVITLYC

SEQ ID NO: 35 - protein, 113 aa, Homo sapiens
  source 1..113: note = CD3zeta signalling domain
SRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 42 aa, Homo sapiens
  source 1..42: note = 4-1BB costimulatory domain
KRGRKKLLYIFKQPFMRPVQTTQEEDGCSCRFPEEEEGGCEL

SEQ ID NO: 37 - protein, 40 aa, Homo sapiens
  source 1..40: note = CD28 costimulatory domain
RSKRSRLLHSDYMNMTPRRPGPTRKHYQPYAPPRDFAAYR

SEQ ID NO: 38 - protein, 17 aa, synthetic construct
  source 1..17: note = leader peptide
MVLILLWLFTAFPGILS

SEQ ID NO: 39 - protein, 676 aa, synthetic construct
  source 1..676: note = JK10
DVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 484 aa, synthetic construct
  source 1..484: note = JK11
DVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 677 aa, synthetic construct
  source 1..677: note = JK15
DVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 483 aa, synthetic construct
  source 1..483: note = JK16
DVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPTTTPAPRPPTPAPTIASQPLSLRPEACRPAAGGAVHTRGLDFACDIYIWAPLAGTCGVLLLSLVITLYCVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 693 aa, synthetic construct
  source 1..693: note = JK10 + leader
MVLILLWLFTAFPGILSDVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 501 aa, synthetic construct
  source 1..501: note = JK11 + leader
MVLILLWLFTAFPGILSDVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 694 aa, synthetic construct
  source 1..694: note = JK15 + leader
MVLILLWLFTAFPGILSDVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWKRGRKKLLYIFKQPFMRPVQTTQEEDGCSCRFPEEEEGGCELSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 500 aa, synthetic construct
  source 1..500: note = JK16 + leader
MVLILLWLFTAFPGILSDVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPTTTPAPRPPTPAPTIASQPLSLRPEACRPAAGGAVHTRGLDFACDIYIWAPLAGTCGVLLLSLVITLYCVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 1253 aa, synthetic construct
  source 1..1253: note = full SwR-JK11 CAR (JK59)
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMVLILLWLFTAFPGILSDVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - DNA, 3759 bp, synthetic construct
  source 1..3759: mol_type = other DNA; note = full SwR-JK11 CAR nt
atggaggctgccgtagctgccccgcgacccagacttctgctgctggtcctcgcagctgctgccgctgcagcagcggcgctgttgcccggcgccaccgccctgcagtgcttttgccacttgtgtaccaaggacaatttcacctgtgtgacagatggtctgtgttttgtgagcgtcacagagactactgataaagtgattcacaatagcatgtgtattgccgaaatagaccttatcccacgtgatcgcccttttgtttgtgcccctagctccaagactggctctgtgaccactacgtattgctgcaaccaagatcattgcaacaaaatcgaactccccaccacagttaaaagcagtcctgggctgggcccggtcgagctcataccttggctgggtcacttgctggtcggcctctcaggcgcctttggtttcatcatactggtgtatctgctgatcaattgccgaaacaccggaccgtggctcaagaaagtgttgaagtgcaacaccccagatccatccaaattcttcagccagttgtcctctgaacatggaggggacgtacagaagtggctttcaagccctttcccctctagcagcttctctcccggcgggttggcaccggagatttctccgctggaggtgctggaacgggataaggtgactcaactccttctgcagcaggacaaggttcctgagccagctagcctgtccagcaaccactcactgacatcctgcttcaccaatcaggggtacttcttctttcatcttcccgatgccctggagatcgaggcttgtcaggtgtactttacgtacgatccttattctgaagaggaccccgacgaaggtgtagctggggcacccacggggagttctccacagcctctccagcctctgtctggggaggatgatgcctactgcacgtttcccagcagggacgacctcctgctctttagcccctccctcctgggaggcccatcacctcccagcacagctcctggaggctcaggcgcaggagaggagagaatgcctcccagtttgcaggaaagagtaccccgcgattgggaccctcagcccctgggaccaccaactcccggagtcccagaccttgttgacttccaacctcctcccgaactggtattgagggaggcaggcgaagaggttccagatgctggtcccagggaaggtgtgagctttccgtggagcagaccaccggggcaaggcgagttcagagccctgaatgcccggctgcctcttaatactgatgcttatctctctctgcaggaactgcagggccaagaccccacacatctggtgggctcaggagaaggcaggggctccctgctcacatgcggggatgtggaagagaacccaggaccaatgggccgcggactgctgcgaggcctttggccactgcatattgtgctctggacacgcattgcgagcaccattccgccacacgtccagaaatctgtgaacaacgacatgattgtcaccgataacaatggggcggtcaaatttcctcaactctgcaagttttgtgacgttcggtttagtacatgcgacaatcagaagtcatgcatgagtaactgtagtatcacctccatctgcgagaaaccacaggaggtctgtgtggcagtttggcggaagaatgacgaaaatattacactggaaaccgtgtgccacgatcctaaactgccttatcatgacttcattttggaggacgctgcctcaccaaagtgtatcatgaaagagaagaagaagcccggtgaaactttcttcatgtgttcctgctcctctgatgagtgtaacgataacatcatcttcagcgaagagtacaatactagtaatcccgacctcctgctggtgatcttccaagtcgtgatatccgtcggaagcatgggcctcataatcagtctgctgtgtgtgtacttttggctggagcggacgatgccacgtattcccacactgaagaacttggaggatcttgtaacagagtaccacgggaacttttcagcatggagcggagtttccaaagggctggcggagagtctgcagcccgactatagcgagaggctgtgtctggtgtccgaaatcccacccaaaggtggtgcccttggcgaaggtcccggagcatccccgtgcaatcagcactctccttattgggcccctccttgttacaccctgaaaccagaaactggaagtggcgccaccaatttctcattgcttaagcaggccggggacgtggaggaaaacccagggcccatggtgctgattcttttgtggctgttcacagcctttcctggtatcctgtctgatgtgcaggttcaggagtcgggacctggcctggtgaaaccttctcagtctctgtccctcacctgcactgtcactggctactcaatcaccagtgattatgcctggaactggattcggcagtttccaggaaacaaactggagtggatgggctacataagtaatagtggtagcactagctacaacccatctctcaaaagtcgaatctctatcactcgagacacatccaagaaccagttcttcctgcagttgatttctgtgactactgaggacacagccacatattactgtgcaagagagaggaactatgattacgacgattattactatgctatggactactggggtcaaggaacctcagtcaccgtctcctcaggaggcggaggctctggcggaggcggctccggaggaggcggctctggcggaggcggcagcgacattgtgatgtcacagtctccatcctccctagctgtgtcagttggagagaaggttaccatgagctgcaagtccagtcagagccttttatataggagcaatcaaaagaactacttggcctggtaccagcagaaaccagggcagtctcctaaactgctgatttattgggcctccactagggaatctggggtccctgatcgcttcacaggcagtggatctgggacagatttcactctcaccatcagcagtgtgaaggctgaagacctggcagtttattactgtcagcaatattataactatcctcggacgttcggtggaggcaccaagctggaaatcaaatcggatcccaccaccaccccagccccacggccacctacccctgccccaaccatcgccagccagcccctgagcctgcggcctgaagcctgcaggcctgccgccggaggagccgtgcacacaaggggcctggacttcgcctgcgacatctatatctgggcccccctggccgggacatgcggggtgctgctgctgtccctggtgattacactgtattgcaaacggggccggaagaagctgctgtacatcttcaagcagcccttcatgcggcccgtgcagaccacccaggaggaggacggctgcagctgccggttccccgaggaagaggaaggcggctgcgagctgcgggtgaagttcagccggagcgccgacgccccagcctaccagcagggccagaaccagctgtacaacgagctgaacctgggacggcgggaggagtacgacgtgctggacaagcggcggggacgggaccccgagatgggcggcaagcctcgccggaagaatccccaggagggcctgtacaacgagctgcagaaggacaagatggccgaggcctacagcgagatcggcatgaagggcgagcggcgccggggcaagggccacgacggcctgtaccagggcctgagcaccgccaccaaggacacctacgacgccctgcacatgcaggccctgccaccccgg

SEQ ID NO: 49 - DNA, 10907 bp, synthetic construct
  source 1..10907: mol_type = other DNA; note = full retroviral vector sequence (JK59)
tgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgggggtggaccatcctctagactgccagcatggaggctgccgtagctgccccgcgacccagacttctgctgctggtcctcgcagctgctgccgctgcagcagcggcgctgttgcccggcgccaccgccctgcagtgcttttgccacttgtgtaccaaggacaatttcacctgtgtgacagatggtctgtgttttgtgagcgtcacagagactactgataaagtgattcacaatagcatgtgtattgccgaaatagaccttatcccacgtgatcgcccttttgtttgtgcccctagctccaagactggctctgtgaccactacgtattgctgcaaccaagatcattgcaacaaaatcgaactccccaccacagttaaaagcagtcctgggctgggcccggtcgagctcataccttggctgggtcacttgctggtcggcctctcaggcgcctttggtttcatcatactggtgtatctgctgatcaattgccgaaacaccggaccgtggctcaagaaagtgttgaagtgcaacaccccagatccatccaaattcttcagccagttgtcctctgaacatggaggggacgtacagaagtggctttcaagccctttcccctctagcagcttctctcccggcgggttggcaccggagatttctccgctggaggtgctggaacgggataaggtgactcaactccttctgcagcaggacaaggttcctgagccagctagcctgtccagcaaccactcactgacatcctgcttcaccaatcaggggtacttcttctttcatcttcccgatgccctggagatcgaggcttgtcaggtgtactttacgtacgatccttattctgaagaggaccccgacgaaggtgtagctggggcacccacggggagttctccacagcctctccagcctctgtctggggaggatgatgcctactgcacgtttcccagcagggacgacctcctgctctttagcccctccctcctgggaggcccatcacctcccagcacagctcctggaggctcaggcgcaggagaggagagaatgcctcccagtttgcaggaaagagtaccccgcgattgggaccctcagcccctgggaccaccaactcccggagtcccagaccttgttgacttccaacctcctcccgaactggtattgagggaggcaggcgaagaggttccagatgctggtcccagggaaggtgtgagctttccgtggagcagaccaccggggcaaggcgagttcagagccctgaatgcccggctgcctcttaatactgatgcttatctctctctgcaggaactgcagggccaagaccccacacatctggtgggctcaggagaaggcaggggctccctgctcacatgcggggatgtggaagagaacccaggaccaatgggccgcggactgctgcgaggcctttggccactgcatattgtgctctggacacgcattgcgagcaccattccgccacacgtccagaaatctgtgaacaacgacatgattgtcaccgataacaatggggcggtcaaatttcctcaactctgcaagttttgtgacgttcggtttagtacatgcgacaatcagaagtcatgcatgagtaactgtagtatcacctccatctgcgagaaaccacaggaggtctgtgtggcagtttggcggaagaatgacgaaaatattacactggaaaccgtgtgccacgatcctaaactgccttatcatgacttcattttggaggacgctgcctcaccaaagtgtatcatgaaagagaagaagaagcccggtgaaactttcttcatgtgttcctgctcctctgatgagtgtaacgataacatcatcttcagcgaagagtacaatactagtaatcccgacctcctgctggtgatcttccaagtcgtgatatccgtcggaagcatgggcctcataatcagtctgctgtgtgtgtacttttggctggagcggacgatgccacgtattcccacactgaagaacttggaggatcttgtaacagagtaccacgggaacttttcagcatggagcggagtttccaaagggctggcggagagtctgcagcccgactatagcgagaggctgtgtctggtgtccgaaatcccacccaaaggtggtgcccttggcgaaggtcccggagcatccccgtgcaatcagcactctccttattgggcccctccttgttacaccctgaaaccagaaactggaagtggcgccaccaatttctcattgcttaagcaggccggggacgtggaggaaaacccagggcccatggtgctgattcttttgtggctgttcacagcctttcctggtatcctgtctgatgtgcaggttcaggagtcgggacctggcctggtgaaaccttctcagtctctgtccctcacctgcactgtcactggctactcaatcaccagtgattatgcctggaactggattcggcagtttccaggaaacaaactggagtggatgggctacataagtaatagtggtagcactagctacaacccatctctcaaaagtcgaatctctatcactcgagacacatccaagaaccagttcttcctgcagttgatttctgtgactactgaggacacagccacatattactgtgcaagagagaggaactatgattacgacgattattactatgctatggactactggggtcaaggaacctcagtcaccgtctcctcaggaggcggaggctctggcggaggcggctccggaggaggcggctctggcggaggcggcagcgacattgtgatgtcacagtctccatcctccctagctgtgtcagttggagagaaggttaccatgagctgcaagtccagtcagagccttttatataggagcaatcaaaagaactacttggcctggtaccagcagaaaccagggcagtctcctaaactgctgatttattgggcctccactagggaatctggggtccctgatcgcttcacaggcagtggatctgggacagatttcactctcaccatcagcagtgtgaaggctgaagacctggcagtttattactgtcagcaatattataactatcctcggacgttcggtggaggcaccaagctggaaatcaaatcggatcccaccaccaccccagccccacggccacctacccctgccccaaccatcgccagccagcccctgagcctgcggcctgaagcctgcaggcctgccgccggaggagccgtgcacacaaggggcctggacttcgcctgcgacatctatatctgggcccccctggccgggacatgcggggtgctgctgctgtccctggtgattacactgtattgcaaacggggccggaagaagctgctgtacatcttcaagcagcccttcatgcggcccgtgcagaccacccaggaggaggacggctgcagctgccggttccccgaggaagaggaaggcggctgcgagctgcgggtgaagttcagccggagcgccgacgccccagcctaccagcagggccagaaccagctgtacaacgagctgaacctgggacggcgggaggagtacgacgtgctggacaagcggcggggacgggaccccgagatgggcggcaagcctcgccggaagaatccccaggagggcctgtacaacgagctgcagaaggacaagatggccgaggcctacagcgagatcggcatgaagggcgagcggcgccggggcaagggccacgacggcctgtaccagggcctgagcaccgccaccaaggacacctacgacgccctgcacatgcaggccctgccaccccggtgaacgcgtatcgatactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttcggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaa

SEQ ID NO: 50 - protein, 1447 aa, synthetic construct
  source 1..1447: note = full SwR-JK15 CAR (JK69)
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMVLILLWLFTAFPGILSDVQVQESGPGLVKPSQSLSLTCTVTGYSITSDYAWNWIRQFPGNKLEWMGYISNSGSTSYNPSLKSRISITRDTSKNQFFLQLISVTTEDTATYYCARERNYDYDDYYYAMDYWGQGTSVTVSSGGGGSGGGGSGGGGSGGGGSDIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYNYPRTFGGGTKLEIKSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - DNA, 4342 bp, synthetic construct
  source 1..4342: mol_type = other DNA; note = full SwR-JK15 CAR (JK69) nt
atggaggctgccgtagctgccccgcgacccagacttctgctgctggtcctcgcagctgctgccgctgcagcagcggcgctgttgcccggcgccaccgccctgcagtgcttttgccacttgtgtaccaaggacaatttcacctgtgtgacagatggtctgtgttttgtgagcgtcacagagactactgataaagtgattcacaatagcatgtgtattgccgaaatagaccttatcccacgtgatcgcccttttgtttgtgcccctagctccaagactggctctgtgaccactacgtattgctgcaaccaagatcattgcaacaaaatcgaactccccaccacagttaaaagcagtcctgggctgggcccggtcgagctcataccttggctgggtcacttgctggtcggcctctcaggcgcctttggtttcatcatactggtgtatctgctgatcaattgccgaaacaccggaccgtggctcaagaaagtgttgaagtgcaacaccccagatccatccaaattcttcagccagttgtcctctgaacatggaggggacgtacagaagtggctttcaagccctttcccctctagcagcttctctcccggcgggttggcaccggagatttctccgctggaggtgctggaacgggataaggtgactcaactccttctgcagcaggacaaggttcctgagccagctagcctgtccagcaaccactcactgacatcctgcttcaccaatcaggggtacttcttctttcatcttcccgatgccctggagatcgaggcttgtcaggtgtactttacgtacgatccttattctgaagaggaccccgacgaaggtgtagctggggcacccacggggagttctccacagcctctccagcctctgtctggggaggatgatgcctactgcacgtttcccagcagggacgacctcctgctctttagcccctccctcctgggaggcccatcacctcccagcacagctcctggaggctcaggcgcaggagaggagagaatgcctcccagtttgcaggaaagagtaccccgcgattgggaccctcagcccctgggaccaccaactcccggagtcccagaccttgttgacttccaacctcctcccgaactggtattgagggaggcaggcgaagaggttccagatgctggtcccagggaaggtgtgagctttccgtggagcagaccaccggggcaaggcgagttcagagccctgaatgcccggctgcctcttaatactgatgcttatctctctctgcaggaactgcagggccaagaccccacacatctggtgggctcaggagaaggcaggggctccctgctcacatgcggggatgtggaagagaacccaggaccaatgggccgcggactgctgcgaggcctttggccactgcatattgtgctctggacacgcattgcgagcaccattccgccacacgtccagaaatctgtgaacaacgacatgattgtcaccgataacaatggggcggtcaaatttcctcaactctgcaagttttgtgacgttcggtttagtacatgcgacaatcagaagtcatgcatgagtaactgtagtatcacctccatctgcgagaaaccacaggaggtctgtgtggcagtttggcggaagaatgacgaaaatattacactggaaaccgtgtgccacgatcctaaactgccttatcatgacttcattttggaggacgctgcctcaccaaagtgtatcatgaaagagaagaagaagcccggtgaaactttcttcatgtgttcctgctcctctgatgagtgtaacgataacatcatcttcagcgaagagtacaatactagtaatcccgacctcctgctggtgatcttccaagtcgtgatatccgtcggaagcatgggcctcataatcagtctgctgtgtgtgtacttttggctggagcggacgatgccacgtattcccacactgaagaacttggaggatcttgtaacagagtaccacgggaacttttcagcatggagcggagtttccaaagggctggcggagagtctgcagcccgactatagcgagaggctgtgtctggtgtccgaaatcccacccaaaggtggtgcccttggcgaaggtcccggagcatccccgtgcaatcagcactctccttattgggcccctccttgttacaccctgaaaccagaaactggaagtggcgccaccaatttctcattgcttaagcaggccggggacgtggaggaaaacccagggcccatggtgctgattcttttgtggctgttcacagcctttcctggtatcctgtctgatgtgcaggttcaggagtcgggacctggcctggtgaaaccttctcagtctctgtccctcacctgcactgtcactggctactcaatcaccagtgattatgcctggaactggattcggcagtttccaggaaacaaactggagtggatgggctacataagtaatagtggtagcactagctacaacccatctctcaaaagtcgaatctctatcactcgagacacatccaagaaccagttcttcctgcagttgatttctgtgactactgaggacacagccacatattactgtgcaagagagaggaactatgattacgacgattattactatgctatggactactggggtcaaggaacctcagtcaccgtctcctcaggaggcggaggctctggcggaggcggctccggaggaggcggctctggcggaggcggcagcgacattgtgatgtcacagtctccatcctccctagctgtgtcagttggagagaaggttaccatgagctgcaagtccagtcagagccttttatataggagcaatcaaaagaactacttggcctggtaccagcagaaaccagggcagtctcctaaactgctgatttattgggcctccactagggaatctggggtccctgatcgcttcacaggcagtggatctgggacagatttcactctcaccatcagcagtgtgaaggctgaagacctggcagtttattactgtcagcaatattataactatcctcggacgttcggtggaggcaccaagctggaaatcaaatcggatcccgccgagcccaaatctcctgacaaaactcacacatgcccaccgtgcccagcacctcccgtggccggcccgtcagtcttcctcttccccccaaaacccaaggacaccctcatgatcgcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggccctgcacaatcactatacccagaaatctctgagtctgagcccaggcaagaaggaccccaagttctgggtcctggtggtggtgggaggcgtgctggcctgttactctctcctggtgaccgtggccttcatcatcttctgggtgaaacggggccggaagaagctgctgtacatcttcaagcagcccttcatgcggcccgtgcagaccacccaggaggaggacggctgcagctgccggttccccgaggaagaggaaggcggctgcgagctgcgggtgaagttcagccggagcgccgacgccccagcctaccagcagggccagaaccagctgtacaacgagctgaacctgggacggcgggaggagtacgacgtgctggacaagcggcggggacgggaccccgagatgggcggcaagcctcgccggaagaatccccaggagggcctgtacaacgagctgcagaaggacaagatggccgaggcctacagcgagatcggcatgaagggcgagcggcgccggggcaagggccacgacggcctgtaccagggcctgagcaccgccaccaaggacacctacgacgccctgcacatgcaggccctgccaccccggt

SEQ ID NO: 52 - DNA, 11489 bp, synthetic construct
  source 1..11489: mol_type = other DNA; note = full retroviral vector sequence (JK69)
tgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgggggtggaccatcctctagactgccagcatggaggctgccgtagctgccccgcgacccagacttctgctgctggtcctcgcagctgctgccgctgcagcagcggcgctgttgcccggcgccaccgccctgcagtgcttttgccacttgtgtaccaaggacaatttcacctgtgtgacagatggtctgtgttttgtgagcgtcacagagactactgataaagtgattcacaatagcatgtgtattgccgaaatagaccttatcccacgtgatcgcccttttgtttgtgcccctagctccaagactggctctgtgaccactacgtattgctgcaaccaagatcattgcaacaaaatcgaactccccaccacagttaaaagcagtcctgggctgggcccggtcgagctcataccttggctgggtcacttgctggtcggcctctcaggcgcctttggtttcatcatactggtgtatctgctgatcaattgccgaaacaccggaccgtggctcaagaaagtgttgaagtgcaacaccccagatccatccaaattcttcagccagttgtcctctgaacatggaggggacgtacagaagtggctttcaagccctttcccctctagcagcttctctcccggcgggttggcaccggagatttctccgctggaggtgctggaacgggataaggtgactcaactccttctgcagcaggacaaggttcctgagccagctagcctgtccagcaaccactcactgacatcctgcttcaccaatcaggggtacttcttctttcatcttcccgatgccctggagatcgaggcttgtcaggtgtactttacgtacgatccttattctgaagaggaccccgacgaaggtgtagctggggcacccacggggagttctccacagcctctccagcctctgtctggggaggatgatgcctactgcacgtttcccagcagggacgacctcctgctctttagcccctccctcctgggaggcccatcacctcccagcacagctcctggaggctcaggcgcaggagaggagagaatgcctcccagtttgcaggaaagagtaccccgcgattgggaccctcagcccctgggaccaccaactcccggagtcccagaccttgttgacttccaacctcctcccgaactggtattgagggaggcaggcgaagaggttccagatgctggtcccagggaaggtgtgagctttccgtggagcagaccaccggggcaaggcgagttcagagccctgaatgcccggctgcctcttaatactgatgcttatctctctctgcaggaactgcagggccaagaccccacacatctggtgggctcaggagaaggcaggggctccctgctcacatgcggggatgtggaagagaacccaggaccaatgggccgcggactgctgcgaggcctttggccactgcatattgtgctctggacacgcattgcgagcaccattccgccacacgtccagaaatctgtgaacaacgacatgattgtcaccgataacaatggggcggtcaaatttcctcaactctgcaagttttgtgacgttcggtttagtacatgcgacaatcagaagtcatgcatgagtaactgtagtatcacctccatctgcgagaaaccacaggaggtctgtgtggcagtttggcggaagaatgacgaaaatattacactggaaaccgtgtgccacgatcctaaactgccttatcatgacttcattttggaggacgctgcctcaccaaagtgtatcatgaaagagaagaagaagcccggtgaaactttcttcatgtgttcctgctcctctgatgagtgtaacgataacatcatcttcagcgaagagtacaatactagtaatcccgacctcctgctggtgatcttccaagtcgtgatatccgtcggaagcatgggcctcataatcagtctgctgtgtgtgtacttttggctggagcggacgatgccacgtattcccacactgaagaacttggaggatcttgtaacagagtaccacgggaacttttcagcatggagcggagtttccaaagggctggcggagagtctgcagcccgactatagcgagaggctgtgtctggtgtccgaaatcccacccaaaggtggtgcccttggcgaaggtcccggagcatccccgtgcaatcagcactctccttattgggcccctccttgttacaccctgaaaccagaaactggaagtggcgccaccaatttctcattgcttaagcaggccggggacgtggaggaaaacccagggcccatggtgctgattcttttgtggctgttcacagcctttcctggtatcctgtctgatgtgcaggttcaggagtcgggacctggcctggtgaaaccttctcagtctctgtccctcacctgcactgtcactggctactcaatcaccagtgattatgcctggaactggattcggcagtttccaggaaacaaactggagtggatgggctacataagtaatagtggtagcactagctacaacccatctctcaaaagtcgaatctctatcactcgagacacatccaagaaccagttcttcctgcagttgatttctgtgactactgaggacacagccacatattactgtgcaagagagaggaactatgattacgacgattattactatgctatggactactggggtcaaggaacctcagtcaccgtctcctcaggaggcggaggctctggcggaggcggctccggaggaggcggctctggcggaggcggcagcgacattgtgatgtcacagtctccatcctccctagctgtgtcagttggagagaaggttaccatgagctgcaagtccagtcagagccttttatataggagcaatcaaaagaactacttggcctggtaccagcagaaaccagggcagtctcctaaactgctgatttattgggcctccactagggaatctggggtccctgatcgcttcacaggcagtggatctgggacagatttcactctcaccatcagcagtgtgaaggctgaagacctggcagtttattactgtcagcaatattataactatcctcggacgttcggtggaggcaccaagctggaaatcaaatcggatcccgccgagcccaaatctcctgacaaaactcacacatgcccaccgtgcccagcacctcccgtggccggcccgtcagtcttcctcttccccccaaaacccaaggacaccctcatgatcgcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggccctgcacaatcactatacccagaaatctctgagtctgagcccaggcaagaaggaccccaagttctgggtcctggtggtggtgggaggcgtgctggcctgttactctctcctggtgaccgtggccttcatcatcttctgggtgaaacggggccggaagaagctgctgtacatcttcaagcagcccttcatgcggcccgtgcagaccacccaggaggaggacggctgcagctgccggttccccgaggaagaggaaggcggctgcgagctgcgggtgaagttcagccggagcgccgacgccccagcctaccagcagggccagaaccagctgtacaacgagctgaacctgggacggcgggaggagtacgacgtgctggacaagcggcggggacgggaccccgagatgggcggcaagcctcgccggaagaatccccaggagggcctgtacaacgagctgcagaaggacaagatggccgaggcctacagcgagatcggcatgaagggcgagcggcgccggggcaagggccacgacggcctgtaccagggcctgagcaccgccaccaaggacacctacgacgccctgcacatgcaggccctgccaccccggtgaacgcgtatcgatactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttcggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaa

SEQ ID NO: 53 - protein, 475 aa, synthetic construct
  source 1..475: note = p95HER2 CAR (JK06)
EVQILETGGGLVKPGGSLRLSCATSGFNFNDYFMNWVRQAPGKGLEWIAQIRNKNYNYATYFAESLEGRFTISRDDSKSSVYLQVNSLRAEDTALYYCTELRYDYWGQGVMVTVSSGGGGSGGGGSAGGGSGGGGSDIVMTQSPFSLAVSEGEMVTINCKSSQSLLSSGNQKNNLAWYQQKPGQSPKLLIYYASTRQSGVPDRFIGSGSGTDFTLTISDVQAEDLADYYCLQHYSSPYTFGAGTKLELKSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 495 aa, synthetic construct
  source 1..495: note = p95HER2 CAR (JK06) + leader
MESQTQALISLLLWVYGTYGEVQILETGGGLVKPGGSLRLSCATSGFNFNDYFMNWVRQAPGKGLEWIAQIRNKNYNYATYFAESLEGRFTISRDDSKSSVYLQVNSLRAEDTALYYCTELRYDYWGQGVMVTVSSGGGGSGGGGSAGGGSGGGGSDIVMTQSPFSLAVSEGEMVTINCKSSQSLLSSGNQKNNLAWYQQKPGQSPKLLIYYASTRQSGVPDRFIGSGSGTDFTLTISDVQAEDLADYYCLQHYSSPYTFGAGTKLELKSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 1248 aa, synthetic construct
  source 1..1248: note = full SwR-p95HER2 CAR (JK63)
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMESQTQALISLLLWVYGTYGEVQILETGGGLVKPGGSLRLSCATSGFNFNDYFMNWVRQAPGKGLEWIAQIRNKNYNYATYFAESLEGRFTISRDDSKSSVYLQVNSLRAEDTALYYCTELRYDYWGQGVMVTVSSGGGGSGGGGSAGGGSGGGGSDIVMTQSPFSLAVSEGEMVTINCKSSQSLLSSGNQKNNLAWYQQKPGQSPKLLIYYASTRQSGVPDRFIGSGSGTDFTLTISDVQAEDLADYYCLQHYSSPYTFGAGTKLELKSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 909 aa, synthetic construct
  source 1..909: note = full Dnm-p95HER2 CAR (JK61)
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLGSGATNFSLLKQAGDVEENPGPMESQTQALISLLLWVYGTYGEVQILETGGGLVKPGGSLRLSCATSGFNFNDYFMNWVRQAPGKGLEWIAQIRNKNYNYATYFAESLEGRFTISRDDSKSSVYLQVNSLRAEDTALYYCTELRYDYWGQGVMVTVSSGGGGSGGGGSAGGGSGGGGSDIVMTQSPFSLAVSEGEMVTINCKSSQSLLSSGNQKNNLAWYQQKPGQSPKLLIYYASTRQSGVPDRFIGSGSGTDFTLTISDVQAEDLADYYCLQHYSSPYTFGAGTKLELKSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - DNA, 1485 bp, synthetic construct
  source 1..1485: mol_type = other DNA; note = p95HER2 CAR (JK06) + leader nt
atggaatcccagacccaagccctgatcagtctgctgctttgggtttacggtacctatggggaggttcagatcctcgagactggaggaggattggtcaaaccaggagggtctttgcgtcttagttgtgccacaagcgggtttaacttcaacgactacttcatgaattgggtaagacaggctcctggcaaagggctggagtggattgcccagattcggaacaagaactacaattacgccacttactttgcggaatccctcgaaggccgattcaccataagtcgggatgacagcaagtcctccgtgtatctgcaagtgaattcactgagagcggaggataccgctctgtattactgcacagagcttcgctacgactattggggccaaggggtgatggtgacagtctcatccggaggcggaggctctggcggtggcggctcagctggtggcggatctggcggaggtggtagcgacatagtcatgacccagtctccctttagcctcgcagtaagcgaaggggagatggtgacgatcaactgcaagtccagccagtcactgctgagtagcgggaatcagaagaacaatctggcatggtatcagcagaaaccaggccagagccctaagctgctgatctattacgctagtactaggcagtcaggggttcccgacaggttcattggctctggctccgggacagacttcacattgacgatcagcgatgtgcaagccgaagatctcgccgattactactgtctgcagcactatagctctccgtatacctttggtgcaggaactaagctggagctcaaatcggatcccaccaccaccccagccccacggccacctacccctgccccaaccatcgccagccagcccctgagcctgcggcctgaagcctgcaggcctgccgccggaggagccgtgcacacaaggggcctggacttcgcctgcgacatctatatctgggcccccctggccgggacatgcggggtgctgctgctgtccctggtgattacactgtattgcaaacggggccggaagaagctgctgtacatcttcaagcagcccttcatgcggcccgtgcagaccacccaggaggaggacggctgcagctgccggttccccgaggaagaggaaggcggctgcgagctgcgggtgaagttcagccggagcgccgacgccccagcctaccagcagggccagaaccagctgtacaacgagctgaacctgggacggcgggaggagtacgacgtgctggacaagcggcggggacgggaccccgagatgggcggcaagcctcgccggaagaatccccaggagggcctgtacaacgagctgcagaaggacaagatggccgaggcctacagcgagatcggcatgaagggcgagcggcgccggggcaagggccacgacggcctgtaccagggcctgagcaccgccaccaaggacacctacgacgccctgcacatgcaggccctgccaccccgg

SEQ ID NO: 58 - DNA, 3744 bp, synthetic construct
  source 1..3744: mol_type = other DNA; note = full SwR-p95HER2 CAR (JK63) nt
atggaggctgccgtagctgccccgcgacccagacttctgctgctggtcctcgcagctgctgccgctgcagcagcggcgctgttgcccggcgccaccgccctgcagtgcttttgccacttgtgtaccaaggacaatttcacctgtgtgacagatggtctgtgttttgtgagcgtcacagagactactgataaagtgattcacaatagcatgtgtattgccgaaatagaccttatcccacgtgatcgcccttttgtttgtgcccctagctccaagactggctctgtgaccactacgtattgctgcaaccaagatcattgcaacaaaatcgaactccccaccacagttaaaagcagtcctgggctgggcccggtcgagctcataccttggctgggtcacttgctggtcggcctctcaggcgcctttggtttcatcatactggtgtatctgctgatcaattgccgaaacaccggaccgtggctcaagaaagtgttgaagtgcaacaccccagatccatccaaattcttcagccagttgtcctctgaacatggaggggacgtacagaagtggctttcaagccctttcccctctagcagcttctctcccggcgggttggcaccggagatttctccgctggaggtgctggaacgggataaggtgactcaactccttctgcagcaggacaaggttcctgagccagctagcctgtccagcaaccactcactgacatcctgcttcaccaatcaggggtacttcttctttcatcttcccgatgccctggagatcgaggcttgtcaggtgtactttacgtacgatccttattctgaagaggaccccgacgaaggtgtagctggggcacccacggggagttctccacagcctctccagcctctgtctggggaggatgatgcctactgcacgtttcccagcagggacgacctcctgctctttagcccctccctcctgggaggcccatcacctcccagcacagctcctggaggctcaggcgcaggagaggagagaatgcctcccagtttgcaggaaagagtaccccgcgattgggaccctcagcccctgggaccaccaactcccggagtcccagaccttgttgacttccaacctcctcccgaactggtattgagggaggcaggcgaagaggttccagatgctggtcccagggaaggtgtgagctttccgtggagcagaccaccggggcaaggcgagttcagagccctgaatgcccggctgcctcttaatactgatgcttatctctctctgcaggaactgcagggccaagaccccacacatctggtgggctcaggagaaggcaggggctccctgctcacatgcggggatgtggaagagaacccaggaccaatgggccgcggactgctgcgaggcctttggccactgcatattgtgctctggacacgcattgcgagcaccattccgccacacgtccagaaatctgtgaacaacgacatgattgtcaccgataacaatggggcggtcaaatttcctcaactctgcaagttttgtgacgttcggtttagtacatgcgacaatcagaagtcatgcatgagtaactgtagtatcacctccatctgcgagaaaccacaggaggtctgtgtggcagtttggcggaagaatgacgaaaatattacactggaaaccgtgtgccacgatcctaaactgccttatcatgacttcattttggaggacgctgcctcaccaaagtgtatcatgaaagagaagaagaagcccggtgaaactttcttcatgtgttcctgctcctctgatgagtgtaacgataacatcatcttcagcgaagagtacaatactagtaatcccgacctcctgctggtgatcttccaagtcgtgatatccgtcggaagcatgggcctcataatcagtctgctgtgtgtgtacttttggctggagcggacgatgccacgtattcccacactgaagaacttggaggatcttgtaacagagtaccacgggaacttttcagcatggagcggagtttccaaagggctggcggagagtctgcagcccgactatagcgagaggctgtgtctggtgtccgaaatcccacccaaaggtggtgcccttggcgaaggtcccggagcatccccgtgcaatcagcactctccttattgggcccctccttgttacaccctgaaaccagaaactggaagtggcgccaccaatttctcattgcttaagcaggccggggacgtggaggaaaacccagggcccatggaatcccagacccaagccctgatcagtctgctgctttgggtttacggtacctatggggaggttcagatcctcgagactggaggaggattggtcaaaccaggagggtctttgcgtcttagttgtgccacaagcgggtttaacttcaacgactacttcatgaattgggtaagacaggctcctggcaaagggctggagtggattgcccagattcggaacaagaactacaattacgccacttactttgcggaatccctcgaaggccgattcaccataagtcgggatgacagcaagtcctccgtgtatctgcaagtgaattcactgagagcggaggataccgctctgtattactgcacagagcttcgctacgactattggggccaaggggtgatggtgacagtctcatccggaggcggaggctctggcggtggcggctcagctggtggcggatctggcggaggtggtagcgacatagtcatgacccagtctccctttagcctcgcagtaagcgaaggggagatggtgacgatcaactgcaagtccagccagtcactgctgagtagcgggaatcagaagaacaatctggcatggtatcagcagaaaccaggccagagccctaagctgctgatctattacgctagtactaggcagtcaggggttcccgacaggttcattggctctggctccgggacagacttcacattgacgatcagcgatgtgcaagccgaagatctcgccgattactactgtctgcagcactatagctctccgtatacctttggtgcaggaactaagctggagctcaaatcggatcccaccaccaccccagccccacggccacctacccctgccccaaccatcgccagccagcccctgagcctgcggcctgaagcctgcaggcctgccgccggaggagccgtgcacacaaggggcctggacttcgcctgcgacatctatatctgggcccccctggccgggacatgcggggtgctgctgctgtccctggtgattacactgtattgcaaacggggccggaagaagctgctgtacatcttcaagcagcccttcatgcggcccgtgcagaccacccaggaggaggacggctgcagctgccggttccccgaggaagaggaaggcggctgcgagctgcgggtgaagttcagccggagcgccgacgccccagcctaccagcagggccagaaccagctgtacaacgagctgaacctgggacggcgggaggagtacgacgtgctggacaagcggcggggacgggaccccgagatgggcggcaagcctcgccggaagaatccccaggagggcctgtacaacgagctgcagaaggacaagatggccgaggcctacagcgagatcggcatgaagggcgagcggcgccggggcaagggccacgacggcctgtaccagggcctgagcaccgccaccaaggacacctacgacgccctgcacatgcaggccctgccaccccgg

SEQ ID NO: 59 - DNA, 2727 bp, synthetic construct
  source 1..2727: mol_type = other DNA; note = full Dnm-p95HER2 CAR (JK61) nt
atggaggctgccgtagctgccccgaggccacgactccttctgctggtgctcgctgctgccgcagccgctgctgcagccttgcttcccggtgccactgcccttcagtgcttctgtcacctgtgcactaaagacaactttacctgtgtcacagatgggctgtgtttcgtcagtgttaccgaaacgacagacaaggtgatacacaacagcatgtgcattgcagaaatagacctgataccacgcgacagaccctttgtgtgtgcgccttccagtaagacaggctctgtaacgaccacgtattgttgcaatcaggatcactgcaataagattgagctgcccactaccgtgaaatcaagcccagggcttgggcctgtcgaactcgccgcagttattgccggtccggtatgtttcgtctgtatctcactcatgctgatggtgtacatctgccataaccgcaccgttatccaccatcgtgtccctaacgaggaagatccctctctggataggcccttcatctctgaaggaggctcaggcgaaggaagagggtccctgctcacatgtggcgatgtagaggagaatccgggacctatgggcaggggtctgttgagaggattgtggcctctgcatattgtgctttggactcggatagctagcacaatcccaccccatgtgcagaaaagcgtgaacaacgatatgatcgtgaccgacaacaatggtgcggtcaaatttccccaactgtgcaagttttgcgacgtgcgcttttccacttgcgacaatcagaaaagctgcatgtccaactgctccattacaagcatctgtgagaaaccacaggaggtttgtgtggccgtttggcgaaagaacgacgagaacattaccctggaaactgtgtgtcacgacccaaagttgccatatcacgatttcattctggaagatgcggcctctccaaagtgcattatgaaggagaagaagaaacccggagagacattcttcatgtgttcctgcagcagtgatgagtgcaatgacaacatcatctttagcgaggaatacaataccagcaatccggatctgctcttggtgatcttccaggtgactgggatttctctgctgcctcccctgggagtagctatctctgtcataattatcttctactgctatcgggtcaatcggcaacagaagctgggctcaggcgctaccaactttagtctgctcaaacaagcaggggacgtggaggaaaacccagggcccatggaatcccagacccaagccctgatcagtctgctgctttgggtttacggtacctatggggaggttcagatcctcgagactggaggaggattggtcaaaccaggagggtctttgcgtcttagttgtgccacaagcgggtttaacttcaacgactacttcatgaattgggtaagacaggctcctggcaaagggctggagtggattgcccagattcggaacaagaactacaattacgccacttactttgcggaatccctcgaaggccgattcaccataagtcgggatgacagcaagtcctccgtgtatctgcaagtgaattcactgagagcggaggataccgctctgtattactgcacagagcttcgctacgactattggggccaaggggtgatggtgacagtctcatccggaggcggaggctctggcggtggcggctcagctggtggcggatctggcggaggtggtagcgacatagtcatgacccagtctccctttagcctcgcagtaagcgaaggggagatggtgacgatcaactgcaagtccagccagtcactgctgagtagcgggaatcagaagaacaatctggcatggtatcagcagaaaccaggccagagccctaagctgctgatctattacgctagtactaggcagtcaggggttcccgacaggttcattggctctggctccgggacagacttcacattgacgatcagcgatgtgcaagccgaagatctcgccgattactactgtctgcagcactatagctctccgtatacctttggtgcaggaactaagctggagctcaaatcggatcccaccaccaccccagccccacggccacctacccctgccccaaccatcgccagccagcccctgagcctgcggcctgaagcctgcaggcctgccgccggaggagccgtgcacacaaggggcctggacttcgcctgcgacatctatatctgggcccccctggccgggacatgcggggtgctgctgctgtccctggtgattacactgtattgcaaacggggccggaagaagctgctgtacatcttcaagcagcccttcatgcggcccgtgcagaccacccaggaggaggacggctgcagctgccggttccccgaggaagaggaaggcggctgcgagctgcgggtgaagttcagccggagcgccgacgccccagcctaccagcagggccagaaccagctgtacaacgagctgaacctgggacggcgggaggagtacgacgtgctggacaagcggcggggacgggaccccgagatgggcggcaagcctcgccggaagaatccccaggagggcctgtacaacgagctgcagaaggacaagatggccgaggcctacagcgagatcggcatgaagggcgagcggcgccggggcaagggccacgacggcctgtaccagggcctgagcaccgccaccaaggacacctacgacgccctgcacatgcaggccctgccaccccgg